SEQUENCE LISTING (55 sequences)

SEQ ID NO: 1 - DNA, 3618 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgatccgatatcgtcgaccaccatggtgatgggcctgggcgttttgttgttggtcttcgtgctgggtctgggtctgaccccaccgaccctggctcaggataactccaggtacacacacttcctgacccagcactatgatgccaaaccacagggccgggatgacagatactgtgaaagcatcatgaggagacggggcctgacctcaccctgcaaagacatcaacacatttattcatggcaacaagcgcagcatcaaggccatctgtgaaaacaagaatggaaaccctcacagagaaaacctaagaataagcaagtcttctttccaggtcaccacttgcaagctacatggagggtccccctggcctccatgccagtaccgagccacagcggggttcagaaacgttgttgttgcttgtgaaaatggcttacctgtccacttggatcagtcaattttccgtcgtccgtaaggtaccgaattcgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 2 - DNA, 4660 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgatatcgtcgaccaccatgacagttttcctttcctttgctttcctcgctgccattctgactcacatagggtgcagcaatcagcgccgaagtccagaaaacagtgggagaagatataaccggattcaacatgggcaatgtgcctacactttcattcttccagaacacgatggcaactgtcgtgagagtacgacagaccagtacaacacaaacgctctgcagagagatgctccacacgtggaaccggatttctcttcccagaaacttcaacatctggaacatgtgatggaaaattatactcagtggctgcaaaaacttgagaattacattgtggaaaacatgaagtcggagatggcccagatacagcagaatgcagttcagaaccacacggctaccatgctggagataggaaccagcctcctctctcagactgcagagcagaccagaaagctgacagatgttgagacccaggtactaaatcaaacttctcgacttgagatacagctgctggagaattcattatccacctacaagctagagaagcaacttcttcaacagacaaatgaaatcttgaagatccatgaaaaaaacagtttattagaacataaaatcttagaaatggaaggaaaacacaaggaagagttggacaccttaaaggaagagaaagagaaccttcaaggcttggttactcgtcaaacatatataatccaggagctggaaaagcaattaaacagagctaccaccaacaacagtgtccttcagaagcagcaactggagctgatggacacagtccacaaccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttgaggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 3 - DNA, 4395 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccgcacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccaccatgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgaaagcttggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 4 - DNA, 3631 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgatatcgtcgaccaccatggctgaaggggaaatcaccaccttcacagccctgaccgagaagtttaatctgcctccagggaattacaagaagcccaaactcctctactgtagcaacgggggccacttcctgaggatccttccggatggcacagtggatgggacaagggacaggagcgaccagcacattcagctgcagctcagtgcggaaagcgtgggggaggtgtatataaagagtaccgagactggccagtacttggccatggacaccgacgggcttttatacggctcacagacaccaaatgaggaatgtttgttcctggaaaggctggaggagaaccattacaacacctatatatccaagaagcatgcagagaagaattggtttgttggcctcaagaagaatgggagctgcaaacgcggtcctcggactcactatggccagaaagcaatcttgtttctccccctgccagtctcttctgattaaggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 5 - DNA, 5643 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgatatcgtcgaccaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactacaagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgaggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 6 - DNA, 5349 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccaccatgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcataggtcgacaagcttggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 7 - DNA, 3425 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatcccaccatgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagaggttcaagatgtgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 8 - DNA, 3922 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgatatcgtcgaccaccatggccacagcaggaaatccctggggctggttcctggggtacctcatccttggtgtcgcaggatcgctcgtctctggtagctgcagccaaatcataaacggcgaggactgcagcccgcactcgcagccctggcaggcggcactggtcatggaaaacgaattgttctgctcgggcgtcctggtgcatccgcagtgggtgctgtcagccgcacactgtttccagaactcctacaccatcgggctgggcctgcacagtcttgaggccgaccaagagccagggagccagatggtggaggccagcctctccgtacggcacccagagtacaacagacccttgctcgctaacgacctcatgctcatcaagttggacgaatccgtgtccgagtctgacaccatccggagcatcagcattgcttcgcagtgccctaccgcggggaactcttgcctcgtttctggctggggtctgctggcgaacggcagaatgcctaccgtgctgcagtgcgtgaacgtgtcggtggtgtctgaggaggtctgcagtaagctctatgacccgctgtaccaccccagcatgttctgcgccggcggagggcacgaccagaaggactcctgcaacggtgactctggggggcccctgatctgcaacgggtacttgcagggccttgtgtctttcggaaaagccccgtgtggccaagttggcgtgccaggtgtctacaccaacctctgcaaattcactgagtggatagagaaaaccgtccaggccagttaagaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 9 - DNA, 4200 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgatatcgtcgaccaccatgagcctcttcgggcttctcctgctgacatctgccctggccggccagagacaggggactcaggcggaatccaacctgagtagtaaattccagttttccagcaacaaggaacagaacggagtacaagatcctcagcatgagagaattattactgtgtctactaatggaagtattcacagcccaaggtttcctcatacttatccaagaaatacggtcttggtatggagattagtagcagtagaggaaaatgtatggatacaacttacgtttgatgaaagatttgggcttgaagacccagaagatgacatatgcaagtatgattttgtagaagttgaggaacccagtgatggaactatattagggcgctggtgtggttctggtactgtaccaggaaaacagatttctaaaggaaatcaaattaggataagatttgtatctgatgaatattttccttctgaaccagggttctgcatccactacaacattgtcatgccacaattcacagaagctgtgagtccttcagtgctacccccttcagctttgccactggacctgcttaataatgctataactgcctttagtaccttggaagaccttattcgatatcttgaaccagagagatggcagttggacttagaagatctatataggccaacttggcaacttcttggcaaggcttttgtttttggaagaaaatccagagtggtggatctgaaccttctaacagaggaggtaagattatacagctgcacacctcgtaacttctcagtgtccataagggaagaactaaagagaaccgataccattttctggccaggttgtctcctggttaaacgctgtggtgggaactgtgcctgttgtctccacaattgcaatgaatgtcaatgtgtcccaagcaaagttactaaaaaataccacgaggtccttcagttgagaccaaagaccggtgtcaggggattgcacaaatcactcaccgacgtggccctggagcaccatgaggagtgtgactgtgtgtgcagagggagcacaggaggataggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 10 - DNA, 3481 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgatatcgtcgaccaccatgagaggtgccacgcgagtctcaatcatgctcctcctagtaactgtgtctgactgtgctgtgatcacaggggcctgtgagcgggatgtccagtgtggggcaggcacctgctgtgccatcagcctgtggcttcgagggctgcggatgtgcaccccgctggggcgggaaggcgaggagtgccaccccggcagccacaagatccccttcttcaggaaacgcaagcaccacacctgtccttgcttgcccaacctgctgtgctccaggttcccggacggcaggtaccgctgctccatggacttgaagaacatcaatttttagcgtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 11 - DNA, 3553 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgatatcgtcgaccaccatgaggagcctgtgctgcgccccactcctgctcctcttgctgctgccgccgctgctgctcacgccccgcgctggggacgccgccgtgatcaccggggcttgtgacaaggactcccaatgtggtggaggcatgtgctgtgctgtcagtatctgggtcaagagcataaggatttgcacacctatgggcaaactgggagacagctgccatccactgactcgtaaaaacaattttggaaatggaaggcaggaaagaagaaagaggaagagaagcaaaaggaaaaaggaggttccattttttgggcggaggatgcatcacacttgcccatgtctgccaggcttggcctgtttacggacttcatttaaccgatttatttgtttagcccaaaagtaaggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 12 - DNA, 34 bp, Artificial Sequence
  Oligonucleotide ANG_F
tttgtcgaccaccatggtgatgggcctgggcgtt

SEQ ID NO: 13 - DNA, 33 bp, Artificial Sequence
  Oligonucleotide ANG_R
aatggtaccttacggacgacggaaaattgactg

SEQ ID NO: 14 - DNA, 20 bp, Artificial Sequence
  Oligonucleotide ANG_SF
tgggcgttttgttgttggtc

SEQ ID NO: 15 - DNA, 20 bp, Artificial Sequence
  Oligonucleotide ANG_FR
tgtctttgcagggtgaggtc

SEQ ID NO: 16 - DNA, 41 bp, Artificial Sequence
  Oligonucleotide ANGPT1_F
tttgtcgaccaccatgacagttttcctttcctttgctttcc

SEQ ID NO: 17 - DNA, 41 bp, Artificial Sequence
  Oligonucleotide ANGPT1_R
aatggtacctcaaaaatctaaaggtcgaatcatcatagttg

SEQ ID NO: 18 - DNA, 20 bp, Artificial Sequence
  Oligonucleotide ANGPT1_SF
tgcagagagatgctccacac

SEQ ID NO: 19 - DNA, 20 bp, Artificial Sequence
  Oligonucleotide ANGPT1_FR
atggtagccgtgtggttctg

SEQ ID NO: 20 - DNA, 38 bp, Artificial Sequence
  Oligonucleotide VEGFA_F
gggggatccaccatgacggacagacagacagacaccgc

SEQ ID NO: 21 - DNA, 37 bp, Artificial Sequence
  Oligonucleotide VEGFA_R
tttggatccaccatgaactttctgctgtcttgggtgc

SEQ ID NO: 22 - DNA, 20 bp, Artificial Sequence
  Oligonucleotide VEGFA_SF
tctgctgtcttgggtgcatt

SEQ ID NO: 23 - DNA, 20 bp, Artificial Sequence
  Oligonucleotide VEGFA_SR
ccagggtctcgattggatgg

SEQ ID NO: 24 - DNA, 34 bp, Artificial Sequence
  Oligonucleotide FGF_F
tttgtcgaccaccatggctgaaggggaaatcacc

SEQ ID NO: 25 - DNA, 33 bp, Artificial Sequence
  Oligonucleotide FGF_R
aatggtaccttaatcagaagagactggcagggg

SEQ ID NO: 26 - DNA, 20 bp, Artificial Sequence
  Oligonucleotide FGF_SF
cagtggatgggacaagggac

SEQ ID NO: 27 - DNA, 20 bp, Artificial Sequence
  Oligonucleotide FGF_FR
ggttctcctccagcctttcc

SEQ ID NO: 28 - DNA, 35 bp, Artificial Sequence
  Oligonucleotide HIF_F
tttgtcgaccaccatggagggcgccggcggcgcga

SEQ ID NO: 29 - DNA, 42 bp, Artificial Sequence
  Oligonucleotide HIF_R
aatggtacctcagttaacttgatccaaagctctgagtaattc

SEQ ID NO: 30 - DNA, 20 bp, Artificial Sequence
  Oligonucleotide HIF_SF
ttttggcagcaacgacacag

SEQ ID NO: 31 - DNA, 20 bp, Artificial Sequence
  Oligonucleotide HIF_FR
gtgcagggtcagcactactt

SEQ ID NO: 32 - DNA, 36 bp, Artificial Sequence
  Oligonucleotide HGF_F
tttggatccaccatgtgggtgaccaaactcctgcca

SEQ ID NO: 33 - DNA, 39 bp, Artificial Sequence
  Oligonucleotide HGF_R
aatgtcgacctatgactgtggtaccttatatgttaaaat

SEQ ID NO: 34 - DNA, 20 bp, Artificial Sequence
  Oligonucleotide HGF_SF
accctggtgtttcacaagca

SEQ ID NO: 35 - DNA, 20 bp, Artificial Sequence
  Oligonucleotide HGF_FR
gcaagaatttgtgccggtgt

SEQ ID NO: 36 - DNA, 31 bp, Artificial Sequence
  Oligonucleotide SDF_F
aggatcccaccatgaacgccaaggtcgtggt

SEQ ID NO: 37 - DNA, 34 bp, Artificial Sequence
  Oligonucleotide SDF_R
tatgaattcacatcttgaacctcttgtttaaagc

SEQ ID NO: 38 - DNA, 20 bp, Artificial Sequence
  Oligonucleotide SDF_SF
tgagctacagatgcccatgc

SEQ ID NO: 39 - DNA, 20 bp, Artificial Sequence
  Oligonucleotide SDF_FR
tagcttcgggtcaatgcaca

SEQ ID NO: 40 - DNA, 34 bp, Artificial Sequence
  Oligonucleotide KLK_F
tttgtcgaccaccatggccacagcaggaaatccc

SEQ ID NO: 41 - DNA, 35 bp, Artificial Sequence
  Oligonucleotide KLK_R
tttttgaattcttaactggcctggacggttttctc

SEQ ID NO: 42 - DNA, 20 bp, Artificial Sequence
  Oligonucleotide KLK_SF
cggagcatcagcattgcttc

SEQ ID NO: 43 - DNA, 20 bp, Artificial Sequence
  Oligonucleotide KLK_FR
gaacatgctggggtggtaca

SEQ ID NO: 44 - DNA, 35 bp, Artificial Sequence
  Oligonucleotide PDGFC_F
tttgtcgaccaccatgagcctcttcgggcttctcc

SEQ ID NO: 45 - DNA, 33 bp, Artificial Sequence
  Oligonucleotide PDGFC_R
aatggtacctatcctcctgtgctccctctgcac

SEQ ID NO: 46 - DNA, 20 bp, Artificial Sequence
  Oligonucleotide PDGFC_SF
atattagggcgctggtgtgg

SEQ ID NO: 47 - DNA, 20 bp, Artificial Sequence
  Oligonucleotide PDGFC_FR
agcactgaaggactcacagc

SEQ ID NO: 48 - DNA, 32 bp, Artificial Sequence
  Oligonucleotide PROK1_F
tatgtcgaccaccatgagaggtgccacgcgag

SEQ ID NO: 49 - DNA, 42 bp, Artificial Sequence
  Oligonucleotide PROK1_R
tatggaattcggtacgctaaaaattgatgttcttcaagtcca

SEQ ID NO: 50 - DNA, 19 bp, Artificial Sequence
  Oligonucleotide PROK1_SF
atcagcctgtggcttcgag

SEQ ID NO: 51 - DNA, 19 bp, Artificial Sequence
  Oligonucleotide PROK1_SR
tcaagtccatggagcagcg

SEQ ID NO: 52 - DNA, 32 bp, Artificial Sequence
  Oligonucleotide PROK2_F
tttgtcgaccaccatgaggagcctgtgctgcg

SEQ ID NO: 53 - DNA, 37 bp, Artificial Sequence
  Oligonucleotide PROK2_R
aatggtaccttacttttgggctaaacaaataaatcgg

SEQ ID NO: 54 - DNA, 20 bp, Artificial Sequence
  Oligonucleotide PROK2_SF
atgggcaaactgggagacag

SEQ ID NO: 55 - DNA, 20 bp, Artificial Sequence
  Oligonucleotide PROK2_SR
atgggcaaactgggagacag

(19) United States
(12) Patent Application Publication
(10) Pub. No.: US 2024/0269324 A1
(43) Pub. Date: Aug. 15, 2024

(54) Gene therapy DNA vector based on gene therapy DNA vector VTvaf17 carrying the therapeutic gene selected from the group of ANG, ANGPT1, VEGFA, FGF1, HIF1a, HGF, SDF1, KLK4, PDGFC, PROK1, and PROK2 genes for increasing the expression level of these therapeutic genes, method of its production and use, Escherichia coli strain SCS110-AF/VTvaf17-ANG, or Escherichia coli strain SCS110-AF/VTvaf17-ANGPT1, or Escherichia coli strain SCS110-AF/VTvaf17-VEGFA, or Escherichia coli strain SCS110-AF/VTvaf17-FGF

(71) Applicants: CELL AND GENE THERAPY LTD, London (GB); OBSCHESTVO S OGRANICHENNOI OTVETSTVENNOSTJU "PRORYVNYE INNOVATSIONNYE TEKHNOLOGII", Moscow (RU)

(72) Inventor: Natalia SAVELIEVA, Wienna (AT)

(21) Appl. No.: 18/269,791

(22) PCT Filed: Dec. 20, 2019
(86) PCT No.: PCT/RU2019/000989

§ 371 Date: Jun. 27, 2023

(30) Foreign Application Priority Data
Dec. 27, 2018 (RU) 2018147085

(51) Int. Cl. A61K 48/00; A61K 38/17; A61K 38/18; A61K 38/46; A61K 38/48; C12N 15/85

(52) U.S. Cl. CPC A61K 48/005; A61K 38/1709; A61K 38/1825; A61K 38/1833; A61K 38/1866; A61K 38/465; A61K 38/4853; C12N 15/85; C12Y 301/27

(57) ABSTRACT

The invention refers to genetic engineering and can be used in biotechnology, medicine, and agriculture for the manufacture of gene therapy products. A gene therapy DNA vector based on the VTvaf1V gene therapy DNA vector is proposed that carries a target gene selected from the group of genes ANG, ANGPT1, VEGFA, FGF1, HIF1a, HGF, SDF1, KFK4, PDGFC, PROK1, PROK2, to increase the expression level of this target gene in humans and animals. Gene therapy DNA vector VTvaf17-ANG or VTvaf 17-AN GPT 1 or VTvaf17-VEGFA or VTvaf17-FGF1 or VTvaf17-HIF1a or VTvaf17-HGF or VTvaf17-SDF1 or VTvaf 17-KFK4 or VTvaf 17-PDGFC, or VTvaf 17-PDKFC or VTvaf17 has the nucleotide sequence of SEQ ID No. 1 or SEQ ID No. 2 or SEQ ID No. 3 or SEQ ID No. 4 or SEQ ID No. 5 or SEQ ID No. 6 or SEQ ID No. 7 or SEQ ID No. 8 or SEQ ID No. 9 or SEQ ID No. 10 or SEQ ID No. 11, respectively. Also provided are a method of producing said vector, the use of a vector, a strain of Escherichia coli carrying said vector, as well as a method of industrial production of said vector.

The invention refers to genetic engineering and can be used in biotechnology, medicine, and agriculture for the manufacture of gene therapy products.

### BACKGROUND OF THE INVENTION

Gene therapy is an innovative approach in medicine aimed at treating inherited and acquired diseases by means of delivery of new genetic material into a patient's cells, tissues, or organs to compensate for or suppress the function of a mutant gene and/or treat a genetic disorder. The objective of gene therapy in most cases is to inject the organism with genes that provide transcription and further translation of protein molecules encoded by these genes. Within the description of the invention, gene expression refers to the production of a protein molecule with amino acid sequence encoded by this gene.

Vascularisation of tissues can be performed via vasculogenesis and angiogenesis. Vasculogenesis is the formation of blood vessels from mesenchymal cells in embryogenesis or endothelial progenitor cells that migrate from the red bone marrow in the postnatal period (postnatal vasculogenesis).

Angiogenesis is the process of new blood vessels formation from the pre-existing vessels. It plays an important role in the development and normal growth of tissues, wound healing processes, the female reproductive cycle (the development of the placenta and the corpus luteum, ovulation), and is involved in pathogenesis of various diseases.

Representatives of the VEGF vascular endothelial growth factor protein family play a key role among vascular invasion promoters. The VEGF family includes 5 representatives: VEGFA, VEGFB, VEGFC, VEGFD. VEGFA binds to the first type VEGF (VEGFR) receptors and VEGFR-2. VEGF-A stimulates proliferation and migration and ensures endothelial cell survival. Most of its effects are due to the activation of VEGFR-2 receptors.

Angiogenin protein, the product of ANG gene, is one of the angiogenesis factors and belongs to RNase A superfamily. Unlike other angiogenesis factors, it demonstrates enzymatic activity toward RNA. Endogenous angiogenin is essential for the cell proliferation processes induced by other proteins, such as VEGF. Similar to VEGF, angiogenin expression may be inducible by hypoxia. The protein encoded by ANG gene is a strong mediator in the new blood vessels formation. The mature peptide has antimicrobial activity towards several bacteria and fungi, including S. pneumoniae and C. albicans. Angiogenin is one of the key proteins involved in the process of angiogenesis in normal and tumour tissues. Angiogenin reacts with actin on the surface of endothelial cells and is transported into the cell nucleus by endocytosis, which further stimulates the processes of cell migration, invasion, and proliferation. Angiogenin is also known to be a follistatin-binding protein. In vivo activity of angiogenin is regulated by RNH1.

Fibroblast growth factors (FGF1, FGF2), interacting with receptors—FGFR-1-4 are strong mitogens for endothelial cells, and also stimulate their migration.

Angiopoietins ANGPT1 and ANGPT2 mediate their action through the Tie-2 receptors of the endothelial cells. ANGPT1 contributes to the survival of endothelial cells, formation of contacts between them, and interaction with pericytes, which stabilises the formed vessels.

Hypoxia-inducible factor (HIF1α) protein—the activity thereof increases with a reduction in oxygen tension in the blood. It was shown that this factor plays an essential role in the bodily response to hypoxia and is synthesised in many tissues of the body, including nervous tissue, where its expression is maximal in neurons. HIF1α is known to induce the transcription of over 60 genes, including VEGF and erythropoietin, that are involved in such biological processes as angiogenesis and erythropoiesis that contribute to the travel and increase in oxygen delivery to the hypoxic areas. This protein also induces transcription of genes involved in cell proliferation and survival, as well as in the glucose and iron metabolism.

Hepatocyte growth factor (HGF) stimulates the regeneration of liver tissue, has a protective effect on hepatocytes and other cells, preventing their apoptosis, and also has an anti-fibrotic effect, inducing the synthesis of extracellular matrix proteinases. HGF stimulates the migration of the resident cardiac stem cells from their localisation to the lesion areas, in particular, in case of myocardial infarction—into the infarction area. DNA containing the natural human gene of hepatocyte growth factor is used for the production of HGF, however, a high level of expression of this protein cannot be obtained when it is used. The sequence of HGF gene optimised for production of high levels of protein is presented in the materials of patent RU 2385936.

Platelet growth factor C (PDGFC) is a protein, one of the many growth factors that plays an important role in angiogenesis. It is found in α-granules in platelets and is synthesised within megakaryocytes. Each platelet contains about a thousand PDGFC molecules. This protein is a strong promoter of tissue repair, with receptors located in the vessel wall on fibroblasts and smooth muscle cells. PDGFC stimulates the proliferation of these cells. In addition, PDGF increases the production of connective tissue components (glycosaminoglycans, collagen, etc.).

Stromal cell-derived factor SDF1 (eng. Stromal cell-derived factor-1) is a chemokine of the CXC subfamily encoded by the CXCL12 gene in humans. SDF1 binds to CXCR4 and CXCR7 receptors and plays an important role in embryonic development and hematopoiesis. SDF1 acts not only as a chemoattractant: in some cases, it can stimulate cell proliferation and promote their survival.

KLK4 kallikrein-like protein belongs to the subgroup of serine proteases with various physiological functions. Scientific evidence suggests that many kallikreins are involved in carcinogenesis, and some of them have potential as new cancer biomarkers and other diseases. This gene is one of the fifteen elements of the kallikrein subfamily located in a cluster on chromosome 19. KLK4 is predominantly expressed in the basal cell nuclei in the epithelium of the prostate in accordance with its distribution in prostate cancer cells in vitro. Kallikreins may promote angiogenesis. Several in vitro studies show that kallikreins support angiogenesis by destroying directly or indirectly the extracellular matrix.

The growth factor secreted by the cells of the endocrine glands is also known and is titled Prokineticin-1 (PROK1). In terms of its structure, this protein is similar to the VEGF family, and it was therefore initially called the endocrine gland-derived vascular endothelial growth factor (EG-VEGF). This molecule induced proliferation, migration, and breaking of membranes in endothelial cells of the capillaries derived from the endocrine glands. However, Prokineticin-1 hardly affected any other types of the tested endothelial and non-endothelial cells. Similar to VEGF, Prokineticin-1 has a HIF-1-binding site, and its expression is induced by hypoxia. Both factors induced massive angiogenesis and the development of ovarian cysts when delivered into the ovaries. However, unlike VEGF, Prokineticin-1 helped to stimulate angiogenesis in the cornea and skeletal muscles. Expression of human PROK1 is found in the cells and tissues of steroidogenic glands, ovaries, testes, adrenal glands and placenta, and often complements the VEGF expression, at that it is assumed that these molecules act concertedly.

Prokineticin-2 protein (PROK2) is a closely related secreted protein to PROK1 that induces proliferation, survival, and migration of endothelial cells of the vessels of adrenal cortex (LeCouter, J. et al., Proc Natl Acad Sci USA 100, 2685-2690 (2003)).

PROK1 (EG-VEGF) and PROK2 (Bv8) proteins are characterised as mitogens selective for specific types of endothelial cells (LeCouter, J. et al., Nature 412(6850):877-84 (2001) and LeCouter, J. et al., Proc Natl Acad Sci USA 100, 2685-2690 (2003)). Other activities are attributed to this family, including nociception (Mollay, C. et al., supra), gastrointestinal motility (Li, M. et al., supra), regulation of daily locomotor activity (Cheng, M. Y., et al., Nature All, 405-410 (2002)) and neurogenesis in the olfactory bulb (Matsumoto, S., et al., Proc Natl Acad Sci USA 103, 4140-4145 (2006)). In addition, Bv8 stimulated the in vitro production of granulocytic and monocytic colonies (LeCouter, J. et al., (2003), supra; Dorsch, M. et al., J. Leukoc Biol 78(2), 426-34 (2005)). Bv8 was characterised as a chemoattractant for macrophages (LeCouter et al., Proc Natl Acad Sci USA.). Genes selected from the group of ANG, ANGPT1, VEGFA, FGF1, HIF1α, HGF, SDF1, KLK4, PDGFC, PROK1, and PROK2 genes play an important role in human and animal organisms.

The correlations between low/insufficient concentrations of these proteins and various diseases in some cases confirmed by disturbances in normal gene expression encoding these proteins was demonstrated.

An increase in the active oxygen production is observed in ANG gene knockout mice compared to the wild type, and hypersensitivity to agents of oxidative stress, hydrogen peroxide, tendency to the reperfusion syndrome development, and cold brain damage, as well as an increased level of mtDNA oxidative damage, structural abnormalities in cardiac myocytes and mitochondria. It is assumed that ANG plays an important role in protecting the cardiac mitochondria from damage under reoxygenation in vivo. Bradykinin-induced vasoconstriction was also observed in mutants. Absence of the ANG allele in transgenic mice enhances certain aspects of aging, namely the level of endothelial dysfunction, vascular remodelling, and leukocyte invasion into cardiovascular tissues. [PMID: 12429206], [PMID: 11579147], [PMID: 14732290], [PMID: 10754271], [PMID: 18760274].

It is known that inhibition of the VEGFA gene function may result in infertility due to the corpus luteum function blocking. Inactivation of a single VEGF allele leads to embryonic death caused by haploinsufficiency due to abnormalities in the development of blood vessels around the 9th day of pregnancy. Differentiation of angioblasts is not impaired, but the formation of vessel lumens, branches and angiogenesis are impaired. VEGF inactivation during postnatal development leads to impaired postnatal vessel development and endothelium viability, increases mortality, retards growth and disrupts the development of the liver, heart, and kidneys (Kozyreva E. V., Davidyan L. Yu. https://www.science-education.ru/ru/article/view?id=20811).

Reduction in expression of a number of kallikrein genes (KLK) has been demonstrated in breast, prostate, and testicular cancers.

In case of genetic defect of SDF-1 in mice, severe disorders of hematopoiesis has been developed as early as during embryogenesis, which is associated with impaired migration of hematopoietic stem cells (HSC) from early hematopoiesis organs (liver, yolk sac) to the bone marrow. Injection of human SDF1 into the spleen and bone marrow of immunodeficient mice leads to rapid homing of introduced human bone marrow cells into the same organs [http://gerontology-explorer.narod.ru/8ead2657-998b-4d77-9c83-8f8f80784343.html].

Thus, the background of the invention suggests that mutations in ANG, ANGPT1, VEGFA, FGF1, HIF1α, HGF, SDF1, KLK4, PDGFC, PROK1, and PROK2 genes or insufficient expression of proteins encoded by these genes are associated with the development of the spectrum of diseases, including, but not limited to, such pathologies as disorders of hematopoiesis, infertility, ischemic myocardial damages, brain damages and damages of muscles of the lower limbs, cancerous tumours, disorders of ontogenesis and neurogenesis, Parkinson's disease, liver fibrosis, pulmonary hypertension, neurodegenerative diseases, in particular, amyotrophic lateral sclerosis (ALS) and other pathological conditions. This is due to the grouping of ANG, ANGPT1, VEGFA, FGF1, HIF1α, HGF, SDF1, KLK4, PDGFC, PROK1, and PROK2 genes into the group of genes within this patent.

Genetic constructs that provide for the expression of proteins encoded by ANG, ANGPT1, VEGFA, FGF1, HIF1α, HGF, SDF1, KLK4, PDGFC, PROK1, and PROK2 genes included in a group of genes as part of a particular vector for gene therapy can be used for drug development for treatment of various diseases, including, but not limited to, such pathologies as disorders of hematopoiesis, infertility, ischemic myocardial damages, brain damages and damages of muscles of the lower limbs, cancerous tumours, disorders of ontogenesis and neurogenesis, Parkinson's disease, liver fibrosis, pulmonary hypertension, neurodegenerative diseases, in particular, amyotrophic lateral sclerosis (ALS) and other pathological conditions.

Moreover, these data suggest that insufficient expression of proteins encoded by ANG, ANGPT1, VEGFA, FGF1, HIF1α, HGF, SDF1, KLK4, PDGFC, PROK1, and PROK2 genes included in the group of genes is associated not only with pathological conditions, but also with a predisposition to their development. Also, these data indicate that insufficient expression of proteins encoded by ANG, ANGPT1, VEGFA, FGF1, HIF1α, HGF, SDF1, KLK4, PDGFC, PROK1, and PROK2 genes may not appear explicitly in the form of a pathology that can be unambiguously described within the framework of existing clinical practice standards (for example, using the ICD code), but at the same time cause conditions that are unfavourable for humans and animals and associated with deterioration in the quality of life.

Thus, an increase in expression of a gene selected from the group of ANG, ANGPT1, VEGFA, FGF1, HIF1α, HGF, SDF1, KLK4, PDGFC, PROK1, and PROK2 genes and introduced into the organism using the gene therapy method is relevant for the correction of conditions of humans and animals associated with the defect of action of the above-mentioned genes.

For the purposes of gene therapy, specially constructed gene therapy vectors divided into viral and non-viral are used. Recently, increasingly more attention is paid to the development of non-viral gene delivery systems with plasmid vectors topping the list. These vectors are free of limitations inherent in viral vectors: in the target cell, they exist as an episome without being integrated into the genome; producing them is quite cheap; there is no immune response or side effects caused by the administration of plasmid vectors, which makes them a convenient tool for gene therapy and prevention of the genetic diseases as DNA vaccination (Li L, Petrovsky N. Molecular mechanisms for enhanced DNA vaccine immunogenicity. Expert Rev Vaccines. 2016; 15(3):313-29).

However, limitations of plasmid vectors use in gene therapy are: 1) presence of antibiotic resistance genes for the production of constructs in carrying strains; 2) the presence of various regulatory elements represented by sequences of viral genomes; 3) size of therapeutic plasmid vector that determines the efficiency of vector delivery to the target cell.

It is known that the European Medicines Agency deems it necessary to refrain from adding antibiotic resistance marker genes to newly engineered plasmid vectors for gene therapy (Reflection paper on design modifications of gene therapy medicinal products during development/14 Dec. 2011 EMA/CAT/GTWP/44236/2009 Committee for advanced therapies) and also recommends avoiding the presence of the regulatory elements in the therapeutic plasmid vectors in order to increase the expression of therapeutic genes (promoters, enhancers, post-translational regulatory elements) that are parts of genomes of various viruses (Guideline on the quality, non-clinical and clinical aspects of gene therapy medicinal products/23 Mar. 2015, EMA/CAT/80183/2014, Committee for Advanced Therapies).

The size of the gene therapy vector is also essential. It is known that modern plasmid vectors often have unnecessary, non-functional sites that increase their length substantially (Mairhofer J, Grabherr R. Rational vector design for efficient non-viral gene delivery: challenges facing the use of plasmid DNA. Mol Biotechnol. 2008.39(2):97-104), which sometimes prevents inserting the therapeutic gene of the desired size into the vector.

A method has been known for accumulating plasmid vectors in Escherichia coli strains without using antibiotics (Cranenburgh R M, Hanak J A, Williams S G, Sherratt D J. Escherichia coli strains that allow antibiotic-free plasmid selection and maintenance by repressor titration. Nucleic Acids Res. 2001. 29(5):E26). DH1lacdapD and DH1lacP2dapD strains of Escherichia coli were constructed, where gene dapD encoding 2,3,4,5-tetrahydropyridine-2,6-dicarboxylate-N-succinyltransferase enzyme involved in the biosynthesis of L-lysine is controlled by the lac promoter. In the absence of the inducer IPTG (Isopropyl-β-D-1-thiogalactopyranoside), these strains are subject to lysis. However, the administration of the pORT multicopy plasmid vector containing the lac operon induces expression of gene dapD, and, therefore, transformed clones may be selected and reproduced. These strains, however, feature low levels and instability of transformation.

An invention is reported in Patent Application No. RU2011152377A for the preparation of an expression plasmid vector without the resistance to antibiotics that contains a polynucleotide sequence encoding the repressor protein. The expression of the said repressor protein regulates the expression of the toxic gene product integrated into the region of the E. coli genome. However, like any other method of selection based on the use of repressor proteins, this method features unstable and inefficient transformation.

U.S. Pat. No. 9,644,211B2 describes a method for producing a vector of the smallest length. This vector does not contain bacterial genome sequences and is produced by parA-mediated recombination in a cultured E. coli strain. The disadvantage of this method of producing the shortest vector is the impossibility to use it on an industrial scale.

The production of gene therapy vectors that include nucleotide sequences encoding human tissue vascularisation proteins is known.

For example, drugs for the treatment of liver fibrosis are known (Gene therapy by hepatocyte growth factor results in regression of experimental liver fibrosis RJGHC.—2010.—Vol. 20.—No 4.—P. 22-28.), in which plasmid constructions containing separately the hepatocyte growth factor (HGF) genes or human urokinase genes are used as the main active substance. The abovementioned plasmid genetic constructs contain protein-coding DNA regions of the appropriate genes and ensure the synthesis and subsequent secretion of hepatocyte growth factor proteins or urokinase from cells as a result of transcription and translation processes when introduced into mammalian cells. The biological activity of appropriate proteins ensures the function support and hepatic cells, hepatocytes division, as well as the disruption of extracellular matrix proteins deposited in the tissue in fibrosis. A method of liver fibrosis treatment is based on multiple-dose intravenous injection of these drugs in amounts of not more than 3.75 mg/kg (for rodents).

Application RU2015117244A describes the use of drug including a mixture of non-viral plasmid constructions pC4W-HGFopt and pVax1-UPAopt containing the HGF and urokinase genes that ensure the synthesis and secretion of the appropriate proteins, the biological activity of which promotes the cure of liver fibrosis when introduced into the liver cells. The claimed drug belongs to the pharmacological class of biological preparations for gene therapy, hepatoprotectors. The intravenous drug administration can stimulate the recovery process of the liver damaged by fibrosis, affecting the survival of hepatocytes, contributing to the destruction of collagen and other protein deposits, replacing the hepatic parenchyma in fibrosis.

Patent No. RF 2491097 describes a pharmaceutical composition for the treatment of neurodegenerative diseases, in particular amyotrophic lateral sclerosis (ALS), containing the adenoviral vector in an effective quantity engineered in the form of a non-replicating nanoparticle based on human adenovirus type 5 genome with human ANG angiogenin gene insertion producing angiogenin and non-replicating nanoparticles in the human organism based on human adenovirus type 5 genome with the vascular endothelial growth factor VEGF gene insertion producing the vascular endothelial growth factor in the human organism. Human angiogenin gene and human vascular endothelial growth factor gene are cloned to two expressing cassettes of a single non-replicating nanoparticle based on human adenovirus type 5 genome. A method for the treatment of ALS is also described that involves injecting a human with a therapeutically effective dose of the indicated pharmaceutical composition.

Patent RF 2522778 describes agent for the treatment of ischemic tissue injuries that constitutes a mixture with a ratio of 1÷0.5−3 from two cultures of mesenchymal stem cells, one of which is modified by the genetic construct based on a viral vector that provides hyperproduction of vascular endothelial growth factor VEGF, and the other is modified by the genetic construct based on a viral vector providing hyperproduction of angiopoietin ANGPT1. A method for the treatment of ischemic tissue injuries is also described that consists in administration by several injections (puncture) directly into ischemic tissue, for example, limb muscles or myocard, in a culture medium devoid of serum, mixtures of cultures of modified mesenchymal stem (stromal) cells that are overproduced with VEGF and ANGPT1 in concentrations from 3 to 100 million cells in 1 ml of solution.

Genetically modified mesenchymal stromal cells of adipose tissue were obtained by transforming these cells with a recombinant adeno-associated virus serotype 2—AAV Helper-Free System (Stratagene, USA) into which the optimised human VEGF 165 gene and the optimised ANGPT1 gene were inserted.

Invention RF No. 2170104 relates to a new method of in vivo presentation and direct transfer of DNA encoding the required repair protein in mammalian repair cells. This method involves implanting a matrix containing the required DNA into a green wound. Repair cells usually found in the vitalised tissue surrounding the wound proliferate and migrate into the matrix activated by the genes, where they collide, absorb, and express DNA. Therefore, transfected repair cells act as in situ bioreactors (localised in the wound) producing agents (RNA, encoded DNA, proteins, etc.) that heal the wound. This invention relates to pharmaceutical compositions that can be used in the embodiment of invention, i.e. when transferring the required DNA. Such compositions include a suitable matrix in combination with the required DNA. DNA molecules can encode different factors that promote wound healing, including extracellular, cell-surface, and intracellular RNA and proteins. For example, hepatocyte growth factor (HGF) gene can be used as the therapeutic gene; platelet growth factor (PDGF) gene; basic fibroblast growth factor genes (FGF1, FGF2, etc.); vascular endothelial growth factor (VEGF) gene, etc.

DNA encoding the required translation or transcription products can be recombinantly integrated into numerous vector systems that provide large-scale replication of DNA in order to produce gene-activated matrices. Vectors used include, but are not limited to, vectors derived from recombinant bacteriophage DNA, plasmid DNA, or cosmid DNA. For example, plasmid vectors such as pBR322, pUC 19/18, pUC 118, 119 and a series of M13 mp vectors can be used. Bacteriophage vectors may include in vivo gene transfer methods for wound healing, patent No. 2170104gt10, in vivo gene transfer methods for wound healing, patent No. 2170104gt11, in vivo gene transfer methods for wound healing, patent No. 2170104gt18-23, in vivo gene transfer methods for wound healing, patent No. 2170104ZAP/R, as well as a series of EMBL bacteriophage vectors. The cosmid vectors used include, but are not limited to, pJB8, pCV 103, pCV 107, pCV 108, pTM, pMCS, pNNL, pHSG274, COS202, COS203, pWE15, pWE16, and a series of charomid 9 vectors. Alternatively, recombinant viral vectors can be constructed, including, but not limited to, vectors derived from viruses such as herpes virus, retroviruses, vaccine viruses, adenoviruses, adeno-associated viruses, or bovine papillomavirus. Although integrating viruses can be used, non-integrating systems not transmitting the gene product to the daughter cells for many generations are preferred for wound healing. Thus, the gene product is expressed during the wound healing process, and while the gene is diluted in subsequent generations the amount of the expressed gene product decreases.

Invention RF No. 2486918 describes a method of stimulating the recovery of peripheral tissue innervation after injury that involves injection of a therapeutically effective amount of a plasmid vector containing the nucleotide sequence SEQ ID NO:1 encoding brain-derived neurotrophic factor (BDNF) or a plasmid vector containing the nucleotide sequence encoding wild-type human urokinase uPA (NM_002658), or combination thereof, or a combination of a plasmid vector containing the nucleotide sequence of SEQ ID NO:1 encoding BDNF with a plasmid vector containing the optimised nucleotide sequence SEQ ID NO:2 encoding stromal cell-derived factor SDF1. The method allows for faster recovery of the structure and conductivity of peripheral nerves after injuries due to the local increase in the production of neurotrophic factors.

In the embodiment of invention, recombinant plasmids containing optimised cDNA (BDNFopt) and human stromal cell-derived factor (SDF-1opt) sequences were constructed for the first time. The best results were obtained using the plasmids pVax1 as the vector (#V260-20, Invitrogen). It is also stated that other plasmid vectors featuring high-copy replication in E. coli and high level of expression of the cloned gene in mammalian cells can be used for cloning. Application No. WO2004081229 provides a description of an invention that offers methods for the application of Bv8 (PROK2) and EG-VEGF (PROK1) polypeptides and corresponding nucleic acids to promote haematopoiesis. It also provides methods of screening for modulators of Bv8 and EG-VEGF activity. Furthermore, the application provides methods of treatment using Bv8 and EG-VEGF polypeptides or Bv8 and EG-VEGF antagonists. Bv8 cloning and expression is described in application WO2003020892.

The prototype of this invention in terms of the use of recombinant DNA vectors for gene therapy is U.S. Pat. No. 9,550,998 (B2) describing the method of producing a recombinant vector for genetic immunisation. The resulting vector is a supercoiled plasmid DNA vector that is used for the expression of cloned genes in human and animal cells. The vector contains an origin of replication (origin), regulatory elements comprising human cytomegalovirus promoter and enhancer, and regulatory sequences from the human T-cell lymphotropic virus. The vector is accumulated in a dedicated E. coli strain free of antibiotics through antisense complementation of sacB gene administered into the strain by means of bacteriophage. The use of this DNA vector in gene therapy is limited by the presence of regulatory sequences of viral genomes.

### DISCLOSURE OF THE INVENTION

The purpose of the invention is to construct gene therapy DNA vectors based on a specially constructed gene therapy DNA vector in order to increase the expression level of a gene selected from the group of the following genes: ANG gene encoding the angiogenin protein, ANGPT1 gene encoding the angiopoietin 1 protein, VEGFA gene encoding the vascular endothelial growth factor protein A, FGF1 gene encoding fibroblast growth factor 1 protein, HIF1α gene encoding hypoxia inducible factor-α protein, HGF gene encoding hepatocyte growth factor protein, SDF1 gene encoding stromal cell-derived factor protein, KLK4 gene encoding the kallikrein-like protein, PDGFC gene encoding platelet growth factor C protein, PROK1 gene encoding prokineticin 1 protein, PROK2 gene encoding prokineticin 2 protein, as well as construction of strains carrying these gene therapy DNA vectors for their production on an industrial scale.

At the same time, DNA vectors must combine the following properties in the optimal way:
I) possibility of safe use in the gene therapy of human beings and animals due to the absence of antibiotic resistance genes in the gene therapy DNA vector,
II) length that ensures efficient gene delivery to the target cell,
III) presence of regulatory elements that ensure efficient expression of the therapeutic genes while not being represented by nucleotide sequences of viral genomes,
IV) producibility and constructability on an industrial scale.

Items I and III are provided herein in compliance with the requirements of the state regulators for gene therapy medicines and, specifically, the requirement of the European Medicines Agency.

The specified purpose is achieved by using the produced gene therapy DNA vector based on the gene therapy DNA vector VTvaf17 for treatment of diseases associated with disorders of tissue vascularisation, angiogenesis and hematopoiesis, hypoxia, disorders of various tissues regeneration, for treatment of diseases including such pathologies as ischemic damage to myocardium, brain, spinal cord, and limb muscle tissues, including in cases of diabetes, oncological and neurodegenerative diseases, including amyotrophic lateral sclerosis while the gene therapy DNA vector VTvaf17-ANG contains the coding region of ANG therapeutic gene cloned to gene therapy DNA vector VTvaf17 with the nucleotide sequence SEQ ID No. 1, the gene therapy DNA vector VTvaf17-ANGPT1 contains the coding region of ANGPT1 therapeutic gene cloned to gene therapy DNA vector VTvaf17 with the nucleotide sequence SEQ ID No. 2, the gene therapy DNA vector VTvaf17-VEGFA contains the coding region of VEGFA therapeutic gene cloned to gene therapy DNA vector VTvaf17 with the nucleotide sequence SEQ ID No. 3, the gene therapy DNA vector VTvaf17-FGF1 contains the coding region of FGF1 therapeutic gene cloned to gene therapy DNA vector VTvaf17 with the nucleotide sequence SEQ ID No. 4, the gene therapy DNA vector VTvaf17-HIF1+a contains the coding region of HIF1α therapeutic gene cloned to gene therapy DNA vector VTvaf17 with the nucleotide sequence SEQ ID No. 5, the gene therapy DNA vector VTvaf17-HGF contains the coding region of HGF therapeutic gene cloned to gene therapy DNA vector VTvaf17 with the nucleotide sequence SEQ ID No. 6, the gene therapy DNA vector VTvaf17-SDF1 contains the coding region of SDF1 therapeutic gene cloned to gene therapy DNA vector VTvaf17 with the nucleotide sequence SEQ ID No. 7, the gene therapy DNA vector VTvaf17-KLK4 contains the coding region of KLK4 therapeutic gene cloned to gene therapy DNA vector VTvaf17 with the nucleotide sequence SEQ ID No. 8, the gene therapy DNA vector VTvaf17-PDGFC contains the coding region of PDGFC therapeutic gene cloned to gene therapy DNA vector VTvaf17 with the nucleotide sequence SEQ ID No. 9, the gene therapy DNA vector VTvaf17-PROK1 contains the coding region of PROK1 therapeutic gene cloned to gene therapy DNA vector VTvaf17 with the nucleotide sequence SEQ ID No. 10, the gene therapy DNA vector VTvaf17-PROK2 contains the coding region of PROK2 therapeutic gene cloned to gene therapy DNA vector VTvaf17 with the nucleotide sequence SEQ ID No. 11.

Each of the constructed gene therapy DNA vectors, namely VTvaf17-ANG, or VTvaf17-ANGPT1, or VTvaf17-VEGFA, or VTvaf17-FGF1, or VTvaf17-HIF1α, or VTvaf17-HGF, or VTvaf17-SDF1, or VTvaf17-KLK4, or VTvaf17-PDGFC, or VTvaf17-PROK1, or VTvaf17-PROK2 due to the limited size of VTvaf17 vector part not exceeding 3200 bp has the ability to efficiently penetrate into human and animal cells and express the ANG, or ANGPT1, or VEGFA, or FGF1, or HIF1α, or HGF, or SDF1, or KLK4, or PDGFC, or PROK1, or PROK2 therapeutic gene cloned to it.

Each of the constructed gene therapy DNA vectors, namely VTvaf17-ANG, or VTvaf17-ANGPT1, or VTvaf17-VEGFA, or VTvaf17-FGF1, or VTvaf17-HIF1α, or VTvaf17-HGF, or VTvaf17-SDF1, or VTvaf17-KLK4, or VTvaf17-PDGFC, or VTvaf17-PROK1, or VTvaf17-PROK2 uses nucleotide sequences that are not antibiotic resistance genes, virus genes, or regulatory elements of viral genomes as the structure elements, which ensures its safe use for gene therapy in humans and animals.

A method of gene therapy DNA vector production based on gene therapy DNA vector VTvaf17 carrying the ANG, ANGPT1, VEGFA, FGF1, HIF1α, HGF, SDF1, KLK4, PDGFC, PROK1, PROK2 therapeutic gene was also developed that involves obtaining each of gene therapy DNA vectors: VTvaf17-ANG, or VTvaf17-ANGPT1, or VTvaf17-VEGFA, or VTvaf17-FGF1, or VTvaf17-HIF1α, or VTvaf17-HGF, or VTvaf17-SDF1, or VTvaf17-KLK4, or VTvaf17-PDGFC, or VTvaf17-PROK1, or VTvaf17-PROK2 as follows: the coding region of the ANG, or ANGPT1, or VEGFA, or FGF1, or HIF1α, or HGF, or SDF1, or KLK4, or PDGFC, or PROK1, or PROK2 therapeutic gene is cloned to gene therapy DNA vector VTvaf17, and gene therapy DNA vector VTvaf17-ANG, SEQ ID No. 1, or VTvaf17-ANGPT1, SEQ ID No. 2, or VTvaf17-VEGFA, SEQ ID No. 3, or VTvaf17-FGF1, SEQ ID No. 4, or VTvaf17-HIF1α, SEQ ID No. 5, or VTvaf17-HGF, SEQ ID No. 6, or VTvaf17-SDF1, SEQ ID No. 7, or VTvaf17-KLK4, SEQ ID No. 8, or VTvaf17-PDGFC, SEQ ID No. 9, or VTvaf17-PROK1, SEQ ID No. 10, or VTvaf17-PROK2, SEQ ID No. 11, respectively, is obtained, while the coding region of the ANG, or ANGPT1, or VEGFA, or FGF1, or HIF1α, or HGF, or SDF1, or KLK4, or PDGFC, or PROK1, or PROK2 therapeutic gene is obtained by isolating total RNA from the human biological tissue sample followed by the reverse transcription reaction and PCR amplification using the obtained oligonucleotides and cleaving the amplification product by corresponding restriction endonucleases, while cloning to the gene therapy DNA vector VTvaf17 is performed by SalI and KpnI, or BamHI and HindIII, or BamHI and SalI, or BamHI and EcoRI, or SalI and EcoRI restriction sites, while the selection is performed without antibiotics,
at the same time, the following oligonucleotides produced for this purpose are used during gene therapy DNA vector VTvaf17-ANG, SEQ ID No. 1 production for the reverse transcription reaction and PCR amplification:

ANG_F
TTTGTCGACCACCATGGTGATGGGCCTGGGCGTT,
ANG_R
AATGGTACCTTACGGACGACGGAAAATTGACTG,

and the cleaving of amplification product and cloning of the coding region of ANG gene to gene therapy DNA vector VTvaf17 is performed by SalI and KpnI restriction endonucleases,
at the same time, the following oligonucleotides produced for this purpose are used during gene therapy DNA vector VTvaf17-ANGPT1, SEQ ID No. 2 production for the reverse transcription reaction and PCR amplification:

ANGPT1_F
TTTGTCGACCACCATGACAGTTTTCCTTTCCTTTGCTTTCC,
ANGPT1_R
AATGGTACCTCAAAAATCTAAAGGTCGAATCATCATAGTTG,

and the cleaving of amplification product and cloning of the coding region of ANGPT1 gene to gene therapy DNA vector VTvaf17 is performed by SalI and KpnI restriction endonucleases,
at the same time, the following oligonucleotides produced for this purpose are used during gene therapy DNA vector VTvaf17-VEGFA, SEQ ID No. 3 production for the reverse transcription reaction and PCR amplification:

VEGFA_F
GGGGGATCCACCATGACGGACAGACAGACAGACACCGC,
VEGFA_R
TTTGGATCCACCATGAACTTTCTGCTGTCTTGGGTGC,

and the cleaving of amplification product and cloning of the coding region of VEGFA gene to gene therapy DNA vector VTvaf17 is performed by BamHI and HindIII restriction endonucleases, at the same time, the following oligonucleotides produced for this purpose are used during gene therapy DNA vector VTvaf17-FGF1, SEQ ID No. 4 production for the reverse transcription reaction and PCR amplification:

FGF_F
TTTGTCGACCACCATGGCTGAAGGGGAAATCACC,
FGF_R
AATGGTACCTTAATCAGAAGAGACTGGCAGGGG,

and the cleaving of amplification product and cloning of the coding region of FGF1 gene to gene therapy DNA vector VTvaf17 is performed by SalI and KpnI restriction endonucleases,
at the same time, the following oligonucleotides produced for this purpose are used during gene therapy DNA vector VTvaf17-HIF1α, SEQ ID No. 5 production for the reverse transcription reaction and PCR amplification:

HIF_F
TTTGTCGACCACCATGGAGGGCGCCGGCGGCGCGA,
HIF_R
AATGGTACCTCAGTTAACTTGATCCAAAGCTCTGAGTAATTC,

and the cleaving of amplification product and cloning of the coding region of HIF1α gene to gene therapy DNA vector VTvaf17 is performed by SalI and KpnI restriction endonucleases,
at the same time, the following oligonucleotides produced for this purpose are used during gene therapy DNA vector VTvaf17-HGF, SEQ ID No. 6 production for the reverse transcription reaction and PCR amplification:

HGF_F
TTTGGATCCACCATGTGGGTGACCAAACTCCTGCCA,
HGF_R
AATGTCGACCTATGACTGTGGTACCTTATATGTTAAAAT,

and the cleaving of amplification product and cloning of the coding region of HGF gene to gene therapy DNA vector VTvaf17 is performed by BamHI and SalI restriction endonucleases,
at the same time, the following oligonucleotides produced for this purpose are used during gene therapy DNA vector VTvaf17-SDF1, SEQ ID No. 7 production for the reverse transcription reaction and PCR amplification:

SDF_F
AGGATCCCACCATGAACGCCAAGGTCGTGGT,
SDF_R
TATGAATTCACATCTTGAACCTCTTGTTTAAAGC,

and the cleaving of amplification product and cloning of the coding region of SDF1 gene to gene therapy DNA vector VTvaf17 is performed by BamHI and EcoRI restriction endonucleases,
at the same time, the following oligonucleotides produced for this purpose are used during gene therapy DNA vector VTvaf17-KLK4, SEQ ID No. 8 production for the reverse transcription reaction and PCR amplification:

KLK_F
TTTGTCGACCACCATGGCCACAGCAGGAAATCCC,
KLK_R
TTTTTGAATTCTTAACTGGCCTGGACGGTTTTCTC,

and the cleaving of amplification product and cloning of the coding region of KLK4 gene to gene therapy DNA vector VTvaf17 is performed by SalI and EcoRI restriction endonucleases,
at the same time, the following oligonucleotides produced for this purpose are used during gene therapy DNA vector VTvaf17-PDGFC, SEQ ID No. 9 production for the reverse transcription reaction and PCR amplification:

PDGFC_F
TTTGTCGACCACCATGAGCCTCTTCGGGCTTCTCC,
PDGFC_R
AATGGTACCTATCCTCCTGTGCTCCCTCTGCAC,

and the cleaving of amplification product and cloning of the coding region of PDGFC gene to gene therapy DNA vector VTvaf17 is performed by SalI and KpnI restriction endonucleases,
at the same time, the following oligonucleotides produced for this purpose are used during gene therapy DNA vector VTvaf17-PROK1, SEQ ID No. 10 production for the reverse transcription reaction and PCR amplification:

PROK1_F
TATGTCGACCACCATGAGAGGTGCCACGCGAG,
PROK1_R
TATGGAATTCGGTACGCTAAAAATTGATGTTCTTCAAGTCCA,

and the cleaving of amplification product and cloning of the coding region of PROK1 gene to gene therapy DNA vector VTvaf17 is performed by SalI and EcoRI restriction endonucleases,
at the same time, the following oligonucleotides produced for this purpose are used during gene therapy DNA vector VTvaf17-PROK2, SEQ ID No. 11 production for the reverse transcription reaction and PCR amplification:

PROK2_F
TTTGTCGACCACCATGAGGAGCCTGTGCTGCG,
PROK2_R
AATGGTACCTTACTTTTGGGCTAAACAAATAAATCGG,

and the cleaving of amplification product and cloning of the coding region of PROK2 gene to gene therapy DNA vector VTvaf17 is performed by SalI and KpnI restriction endonucleases.

A method of use of the gene therapy DNA vector based on gene therapy DNA vector VTvaf17 carrying ANG, ANGPT1, VEGFA, FGF1, HIF1α, HGF, SDF1, KLK4, PDGFC, PROK1, and PROK2, therapeutic gene for treatment of diseases associated with disorders of tissue vascularisation, angiogenesis and hematopoiesis, hypoxia, disorders of various tissues regeneration, for treatment of diseases including such pathologies as ischemic damage to myocardium, brain, spinal cord, and limb muscle tissues, including in cases of diabetes, oncological and neurodegenerative diseases, including amyotrophic lateral sclerosis was developed that involves transfection of the cells of patient or animal organs and tissues with the selected gene therapy DNA vector carrying the therapeutic gene based on gene therapy DNA vector VTvaf17, or several selected gene therapy DNA vectors carrying therapeutic genes based on gene therapy DNA vector VTvaf17, from the group of constructed gene therapy DNA vectors carrying therapeutic genes based on gene therapy DNA vector VTvaf17 and/or injection of autologous cells of said patient or animal transfected by the selected gene therapy DNA vector carrying therapeutic gene based on gene therapy DNA vector VTvaf17 or several selected gene therapy DNA vectors carrying the therapeutic genes based on gene therapy DNA vector VTvaf17 from the constructed gene therapy DNA vectors carrying therapeutic genes based on gene therapy DNA vector VTvaf17 into the organs and tissues of the same patient or animal and/or the injection of the selected gene therapy DNA vector carrying therapeutic gene based on gene therapy DNA vector VTvaf17 or several selected gene therapy DNA vectors carrying therapeutic genes based on gene therapy DNA vector VTvaf17 from the group of constructed gene therapy DNA vectors carrying therapeutic genes based on gene therapy DNA vector VTvaf17 into the organs and tissues of the same patient or animal, or the combination of the indicated methods.

A method of production of strain for construction of a gene therapy DNA vector for treatment of diseases associated with disorders of tissue vascularisation, angiogenesis and hematopoiesis, hypoxia, disorders of various tissues regeneration, for treatment of diseases including such pathologies as ischemic damage to myocardium, brain, spinal cord, and limb muscle tissues, including in cases of diabetes, oncological and neurodegenerative diseases, including amyotrophic lateral sclerosis was developed that involves making electrocompetent cells of Escherichia coli strain SCS110-AF and subjecting these cells to electroporation with gene therapy DNA vector VTvaf17-ANG, or gene therapy DNA vector VTvaf17-ANGPT1, or gene therapy DNA vector VTvaf17-VEGFA, or gene therapy DNA vector VTvaf17-FGF1, or gene therapy DNA vector VTvaf17-HIF1α, or gene therapy DNA vector VTvaf17-HGF, or gene therapy DNA vector VTvaf17-SDF1, or gene therapy DNA vector VTvaf17-KLK4, gene therapy DNA vector VTvaf17-PDGFC, or gene therapy DNA vector VTvaf17-PROK1, or gene therapy DNA vector VTvaf17-PROK2. After that, the cells are poured into agar plates (Petri dishes) with a selective medium containing yeastrel, peptone, 6% sucrose, and 10 μg/ml of chloramphenicol, and as a result, Escherichia coli strain SCS110-AF/VTvaf17-ANG, or Escherichia coli strain SCS110-AF/VTvaf17-ANGPT1, or Escherichia coli strain SCS110-AF/VTvaf17-VEGFA, or Escherichia coli strain SCS110-AF/VTvaf17-FGF1, or Escherichia coli strain SCS110-AF/VTvaf17-HIF1α, or Escherichia coli strain SCS110-AF/VTvaf17-HGF, or Escherichia coli strain SCS110-AF/VTvaf17-SDF1, or Escherichia coli strain SCS110-AF/VTvaf17-KLK4, or Escherichia coli strain SCS110-AF/VTvaf17-PDGFC, or Escherichia coli strain SCS110-AF/VTvaf17-PROK1, or Escherichia coli strain SCS110-AF/VTvaf17-PROK2 is obtained.

Escherichia coli strain SCS110-AF/VTvaf17-ANG carrying the gene therapy DNA vector VTvaf17-ANG for production thereof allowing for antibiotic-free selection during gene therapy DNA vector production, or Escherichia coli strain SCS110-AF/VTvaf17-ANGPT1 carrying the gene therapy DNA vector VTvaf17-ANGPT1 for production thereof allowing for antibiotic-free selection during gene therapy DNA vector production, or Escherichia coli strain SCS110-AF/VTvaf17-VEGFA carrying the gene therapy DNA vector VTvaf17-VEGFA for production thereof allowing for antibiotic-free selection during gene therapy DNA vector production, or Escherichia coli strain SCS110-AF/VTvaf17-FGF1 carrying the gene therapy DNA vector VTvaf17-FGF1 for production thereof allowing for antibiotic-free selection during gene therapy DNA vector production, or Escherichia coli strain SCS110-AF/VTvaf17-HIF1α carrying the gene therapy DNA vector VTvaf17-HIF1α for production thereof allowing for antibiotic-free selection during gene therapy DNA vector production, or Escherichia coli strain SCS110-AF/VTvaf17-HGF carrying the gene therapy DNA vector VTvaf17-HGF for production thereof allowing for antibiotic-free selection during gene therapy DNA vector production, or Escherichia coli strain SCS110-AF/VTvaf17-SDF1 carrying the gene therapy DNA vector VTvaf17-SDF1 for production thereof allowing for antibiotic-free selection during gene therapy DNA vector production, or Escherichia coli strain SCS110-AF/VTvaf17-KLK4 carrying the gene therapy DNA vector VTvaf17-KLK4 for production thereof allowing for antibiotic-free selection during gene therapy DNA vector production, or Escherichia coli strain SCS110-AF/VTvaf17-PDGFC carrying the gene therapy DNA vector VTvaf17-PDGFC for production thereof allowing for antibiotic-free selection during gene therapy DNA vector production, or Escherichia coli strain SCS110-AF/VTvaf17-PROK1 carrying the gene therapy DNA vector VTvaf17-PROK1 for production thereof allowing for antibiotic-free selection during gene therapy DNA vector production, or Escherichia coli strain SCS110-AF/VTvaf17-PROK2 carrying the gene therapy DNA vector VTvaf17-PROK2 for production thereof allowing for antibiotic-free selection during gene therapy DNA vector production is claimed for treatment of diseases associated with disorders of tissue vascularisation, angiogenesis and hematopoiesis, hypoxia, disorders of various tissues regeneration, for treatment of diseases including such pathologies as ischemic damage to myocardium, brain, spinal cord, and limb muscle tissues, including in cases of diabetes, oncological and neurodegenerative diseases, including amyotrophic lateral sclerosis.

A method of production on an industrial scale of gene therapy DNA vector based on gene therapy DNA vector VTvaf17 carrying the ANG, or ANGPT1, or VEGFA, or FGF1, or HIF1α, or HGF, or SDF1, or KLK4, or PDGFC, or PROK1, or PROK2 therapeutic gene for treatment of diseases associated with disorders of tissue vascularisation, angiogenesis and hematopoiesis, hypoxia, disorders of various tissues regeneration, for treatment of diseases including such pathologies as ischemic damage to myocardium, brain, spinal cord, and limb muscle tissues, including in cases of diabetes, oncological and neurodegenerative diseases, including amyotrophic lateral sclerosis was developed that involves production of gene therapy DNA vector VTvaf17-ANG, or gene therapy DNA vector VTvaf17-ANGPT1, or gene therapy DNA vector VTvaf17-VEGFA, or gene therapy DNA vector VTvaf17-FGF1, or gene therapy DNA vector VTvaf17-HIF1α, or gene therapy DNA vector VTvaf17-HGF, or gene therapy DNA vector VTvaf17-SDF1, or gene therapy DNA vector VTvaf17-KLK4, or gene therapy DNA vector VTvaf17-PDGFC, or gene therapy DNA vector VTvaf17-PROK1, or gene therapy DNA vector VTvaf17-PROK2 by inoculating a culture flask containing the prepared medium with seed culture selected from Escherichia coli strain SCS110-AF/VTvaf17-ANG, or Escherichia coli strain SCS110-AF/VTvaf17-ANGPT1, or Escherichia coli strain SCS110-AF/VTvaf17-VEGFA, or Escherichia coli strain SCS110-AF/VTvaf17-FGF1, or Escherichia coli strain SCS110-AF/VTvaf17-HIF1α, or Escherichia coli strain SCS110-AF/VTvaf17-HGF, or Escherichia coli strain SCS110-AF/VTvaf17-SDF1, or Escherichia coli strain SCS110-AF/VTvaf17-KLK4, or Escherichia coli strain SCS110-AF/VTvaf17-PDGFC, or Escherichia coli strain SCS110-AF/VTvaf17-PROK1, or Escherichia coli strain SCS110-AF/VTvaf17-PROK2, then the cell culture is incubated in an incubator shaker and transferred to an industrial fermenter, then grown to a stationary phase, then the fraction containing the target DNA product is extracted, multi-stage filtered, and purified by chromatographic methods.

The essence of the invention is explained in the drawings, where:

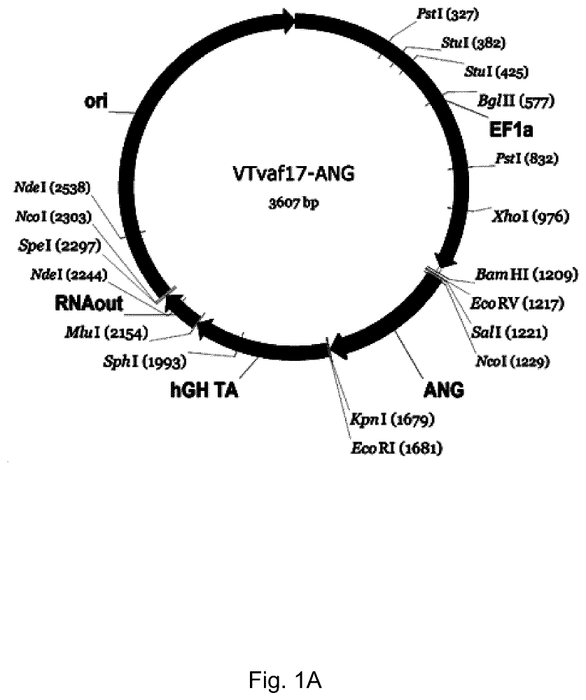

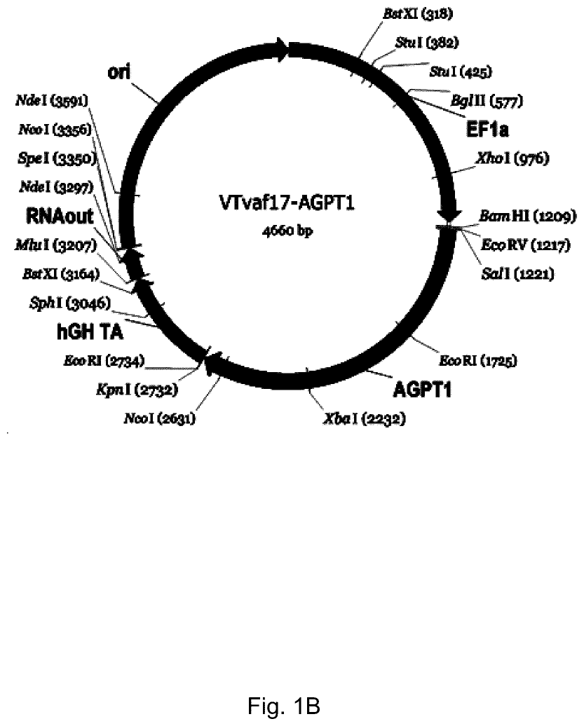

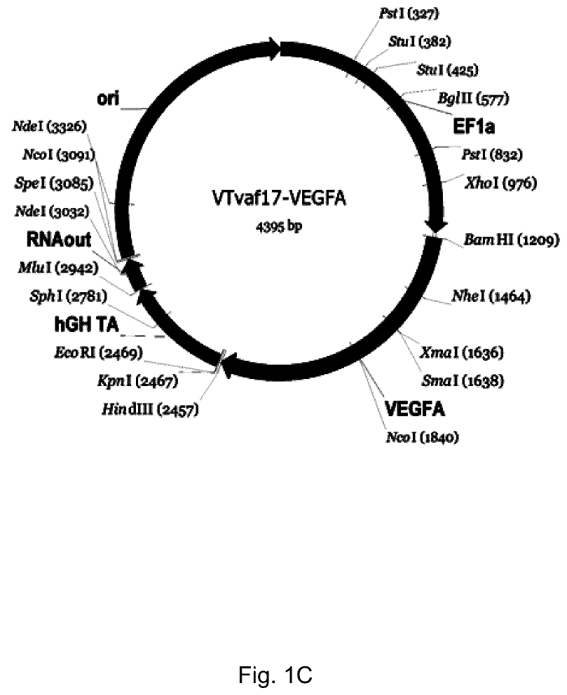

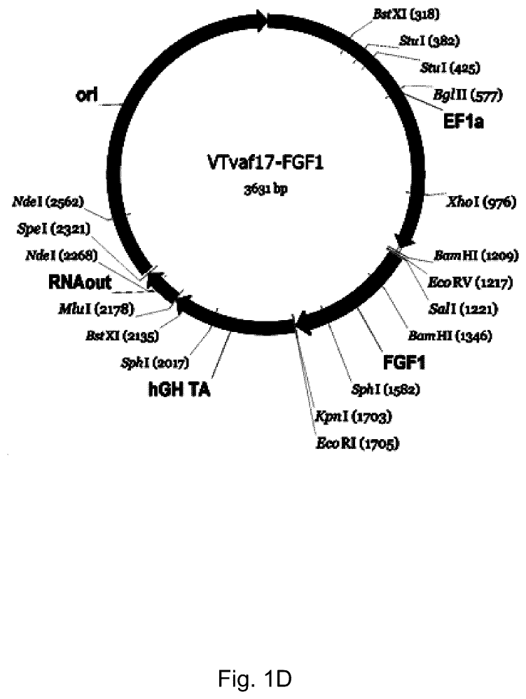

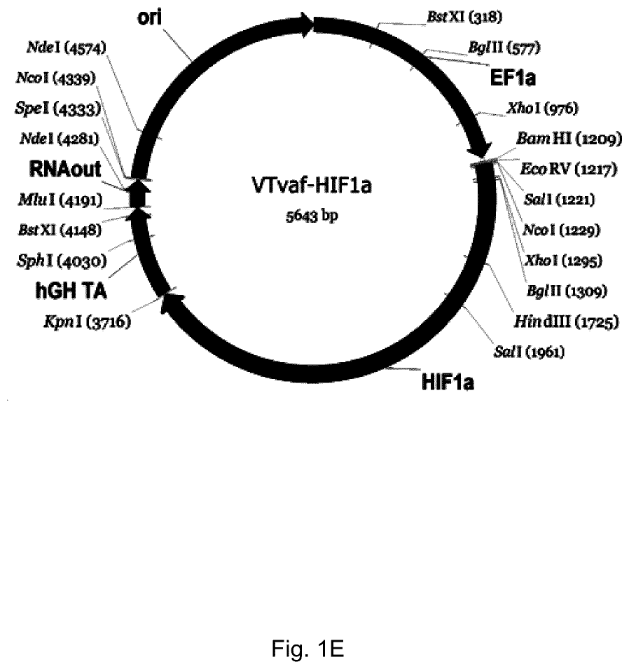

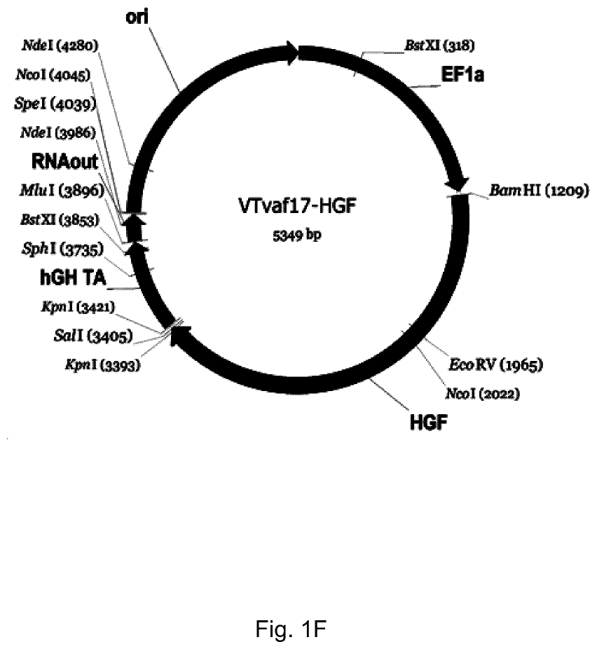

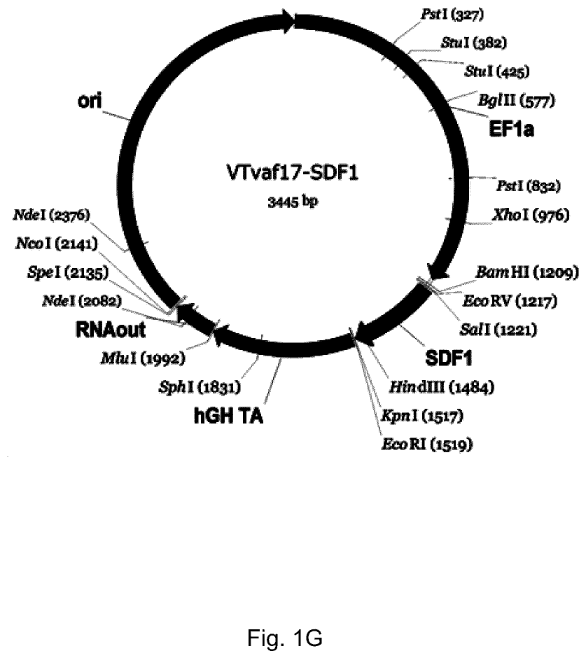

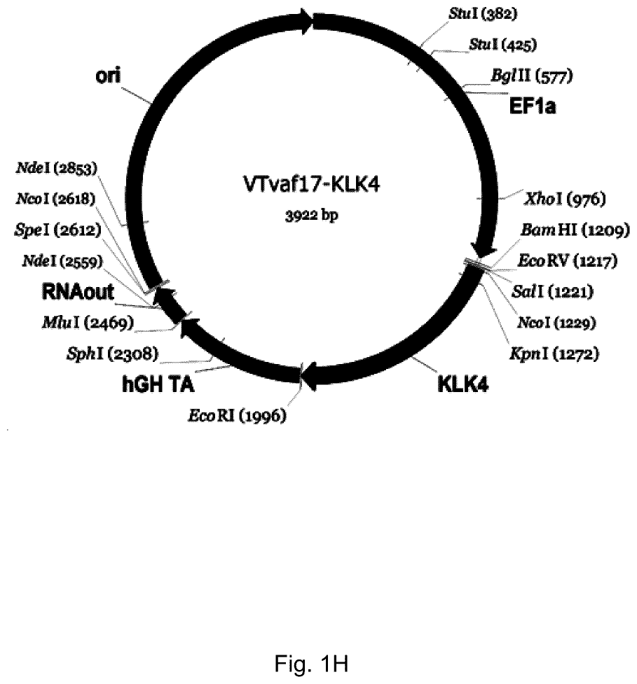

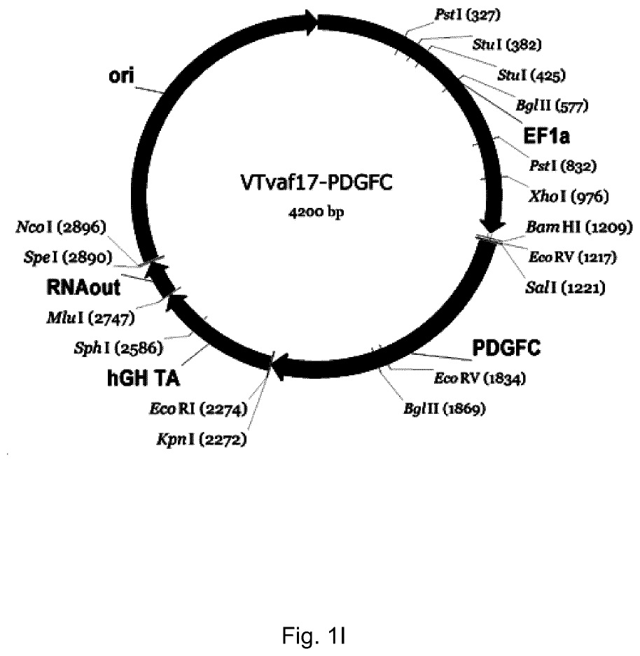

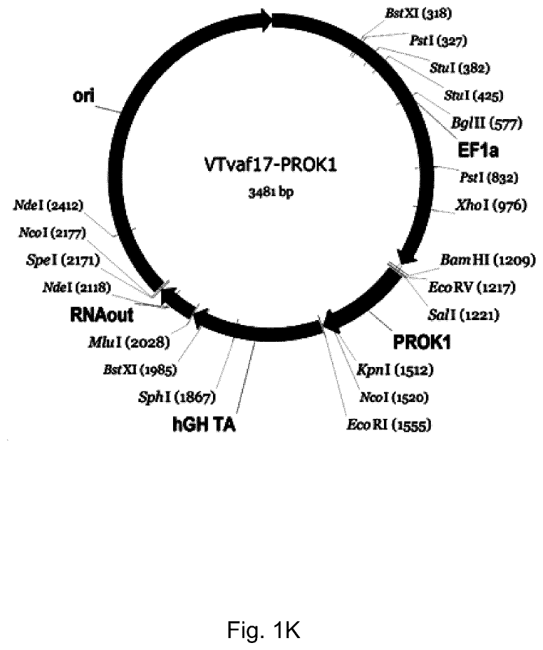

FIG. 1
shows the structure of gene therapy DNA vector VTvaf17 carrying cDNA of the human therapeutic gene selected from the group of ANG, ANGPT1, VEGFA, FGF1, HIF1α, HGF, SDF1, KLK4, PDGFC, PROK1, and PROK2 genes that constitutes a circular double-stranded DNA molecule capable of autonomous replication in Escherichia coli cells.

FIG. 1 shows the structures corresponding to:
A—gene therapy DNA vector VTvaf17-ANG,
B—gene therapy DNA vector VTvaf17-ANGPT1,
C—gene therapy DNA vector VTvaf17-VEGFA,
D—gene therapy DNA vector VTvaf17-FGF1,
E—gene therapy DNA vector VTvaf17-HIF1α,
F—gene therapy DNA vector VTvaf17-HGF,
G—gene therapy DNA vector VTvaf17-SDF1,
H—gene therapy DNA vector VTvaf17-KLK4,
I—gene therapy DNA vector VTvaf17-PDGFC,
K—gene therapy DNA vector VTvaf17-PROK1,
L—gene therapy DNA vector VTvaf17-PROK2.
The following structural elements of the vector are indicated in the structures:
EF1a—the promoter region of human elongation factor EF1A with an intrinsic enhancer contained in the first intron of the gene. It ensures efficient transcription of the recombinant gene in most human tissues.
The reading frame of the therapeutic gene corresponding to the coding region of the ANG gene (FIG. 1A), or ANGPT1 (FIG. 1B), or VEGFA (FIG. 1C), or FGF1 (FIG. 1D), or HIF1α (FIG. 1E), or HGF (FIG. 1F), or SDF1 (FIG. 1G), or KLK4 (FIG. 1H), or PDGFC (FIG. 1I), or PROK1 (FIG. 1K), or PROK2 (FIG. 1L), respectively;
hGH TA—the transcription terminator and the polyadenylation site of the human growth factor gene,
(4) RNAout—the regulatory element RNA-OUT of transposon Tn 10 allowing for antibiotic-free positive selection in case of the use of E. coli strain SCS110-AF,
ori—the origin of replication, the site for autonomous replication with a single nucleotide substitution to increase plasmid production in the cells of most E. coli strains.
Unique restriction sites of Escherichia coli are marked.

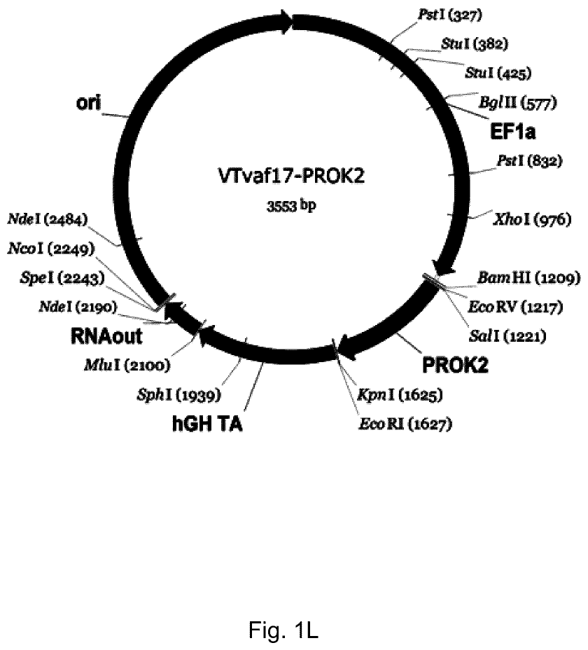

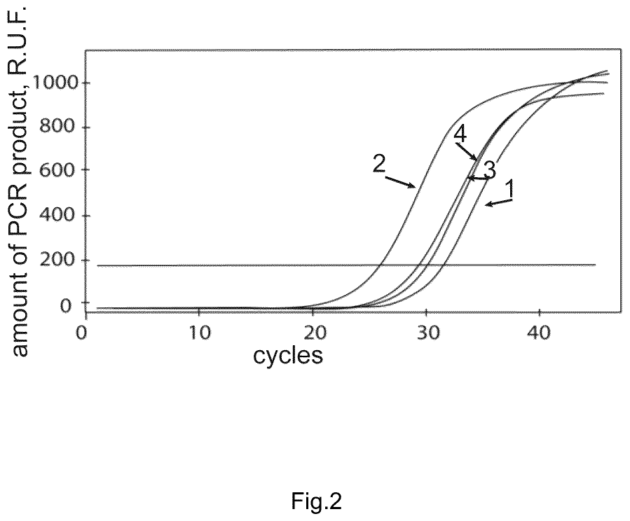

FIG. 2
shows diagrams of mRNA accumulation of the therapeutic gene, namely the human ANG gene, in HDFa human primary dermal fibroblast cells (ATCCPCS-201-012) before their transfection and 48 hours after transfection of these cells with the DNA vector VVTvaf17-ANG in order to confirm the efficiency of gene therapy DNA vector VTvaf17-ANG carrying the ANG therapeutic gene, where
1—cDNA of ANG gene before transfection with gene therapy DNA vector VTvaf17-ANG,
2—cDNA of ANG gene after transfection with gene therapy DNA vector VTvaf17-ANG,
3—cDNA of B2M gene before transfection with gene therapy DNA vector VTvaf17-ANG,
4—cDNA of B2M gene after transfection with gene therapy DNA vector VTvaf17-ANG.
B2M (beta-2-microglobuline) gene listed in the GenBank database under number NM 004048.2 was used as a reference gene.

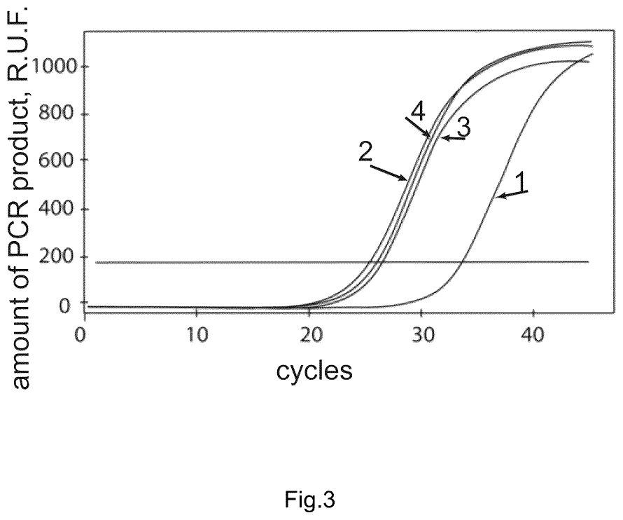

FIG. 3
shows diagrams of mRNA accumulation of the therapeutic gene, namely the human ANGPT1 gene, in HT 297.T human fibroblast cells before their transfection and 48 hours after transfection of these cells with the DNA vector VTvaf17-ANGPT1 in order to confirm the efficiency of gene therapy DNA vector VTvaf17-ANGPT1 carrying the ANGPT1 therapeutic gene, where
1—cDNA of ANGPT1 gene before transfection with gene therapy DNA vector VTvaf17-ANGPT1,
2—cDNA of ANGPT1 gene after transfection with gene therapy DNA vector VTvaf17-ANGPT1,
3—cDNA of B2M gene before transfection with gene therapy DNA vector VTvaf17-ANGPT1,
4—cDNA of B2M gene after transfection with gene therapy DNA vector VTvaf17-ANGPT1.
B2M (beta-2-microglobuline) gene listed in the GenBank database under number NM 004048.2 was used as a reference gene.

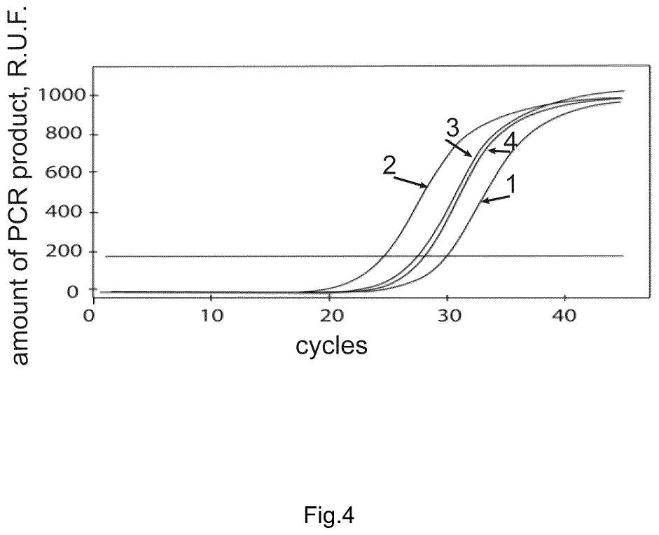

FIG. 4
shows diagrams of mRNA accumulation of the therapeutic gene, namely the human VEGFA gene, in Hs27 human foreskin fibroblast culture cells before their transfection and 48 hours after transfection of these cells with the DNA vector VTvaf17-VEGFA in order to confirm the efficiency of gene therapy DNA vector VTvaf17-VEGFA carrying the VEGFA therapeutic gene, where
1—cDNA of VEGFA gene before transfection with gene therapy DNA vector VTvaf17-VEGFA,
2—cDNA of VEGFA gene after transfection with gene therapy DNA vector VTvaf17-VEGFA,
3—cDNA of B2M gene before transfection with gene therapy DNA vector VTvaf17-VEGFA,
4—cDNA of B2M gene after transfection with gene therapy DNA vector VTvaf17-VEGFA.
B2M (beta-2-microglobuline) gene listed in the GenBank database under number NM 004048.2 was used as a reference gene.

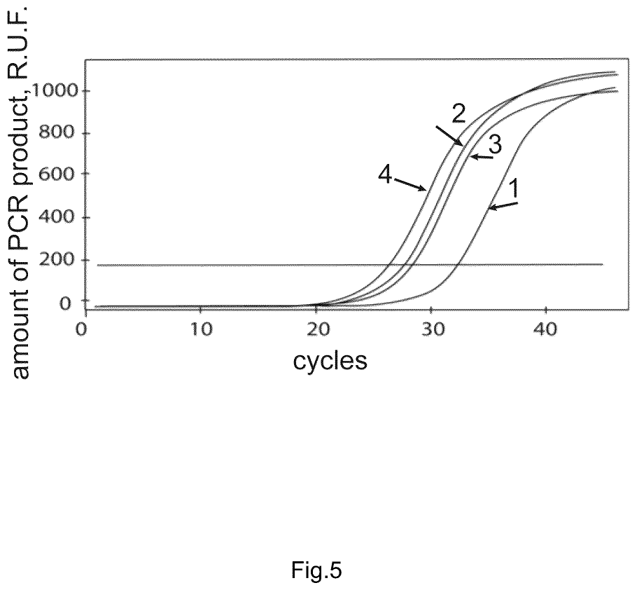

FIG. 5
shows diagrams of mRNA accumulation of the therapeutic gene, namely the human FGF1 gene, in HSkM human skeletal muscle myoblast culture cells before their transfection and 48 hours after transfection of these cells with the DNA vector VTvaf17-FGF1 in order to confirm the efficiency of gene therapy DNA vector VTvaf17-FGF1 carrying the FGF1 therapeutic gene, where
1—cDNA of FGF1 gene before transfection with gene therapy DNA vector VTvaf17-FGF1,
2—cDNA of FGF1 gene after transfection with gene therapy DNA vector VTvaf17-FGF1,
3—cDNA of B2M gene before transfection with gene therapy DNA vector VTvaf17-FGF1,
4—cDNA of B2M gene in cells after transfection with gene therapy DNA vector VTvaf17-FGF1.
B2M (beta-2-microglobuline) gene listed in the GenBank database under number NM 004048.2 was used as a reference gene.

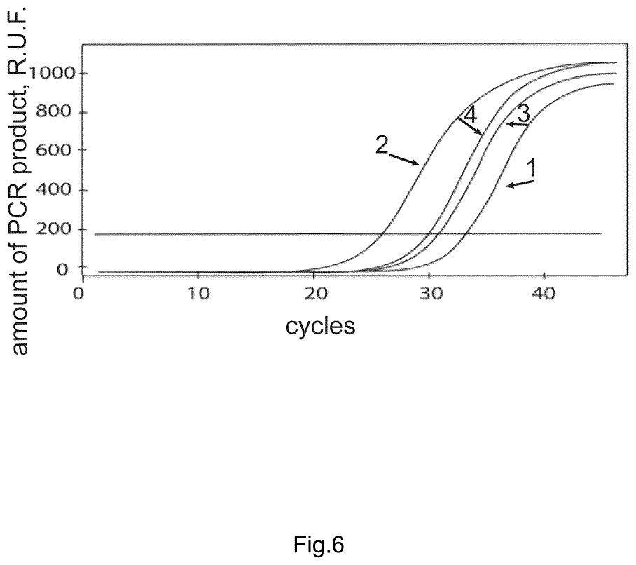

FIG. 6
shows diagrams of mRNA accumulation of the therapeutic gene, namely the human HIF1α gene, in the HBdSMc human urinary bladder smooth muscle culture before its transfection and 48 hours after transfection of these cells with the DNA vector VTvaf17-HIF1α in order to confirm the efficiency of gene therapy DNA vector VTvaf17-HIF1α carrying the HIF1α therapeutic gene, where
1—cDNA of HIF1α gene before transfection with gene therapy DNA vector VTvaf17-HIF1α,
2—cDNA of HIF1α gene after transfection with gene therapy DNA vector VTvaf17-HIF1α,
3—cDNA of B2M gene before transfection with gene therapy DNA vector VTvaf17-HIF1α,
4—cDNA of B2M gene in cells after transfection with gene therapy DNA vector VTvaf17-HIF1α.
B2M (beta-2-microglobuline) gene listed in the GenBank database under number NM 004048.2 was used as a reference gene.

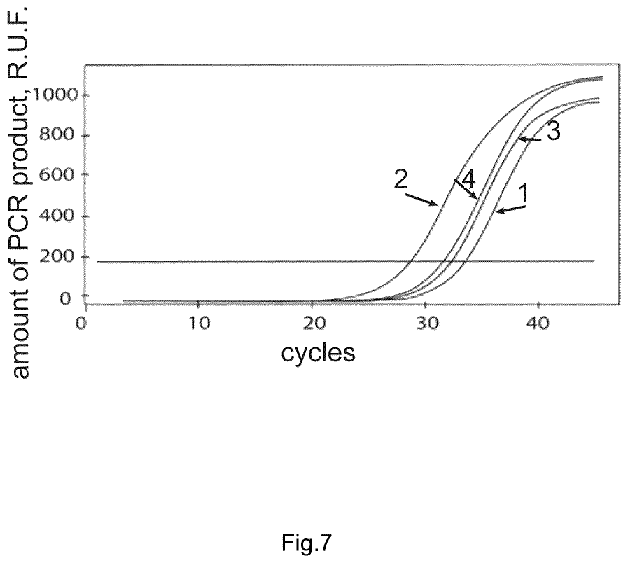

FIG. 7
shows diagrams of mRNA accumulation of the therapeutic gene, namely the human HGF gene, in T/GHA VSMC human aortic smooth muscle cell culture before its transfection and 48 hours after transfection of these cells with the DNA vector VTvaf17-HGF in order to confirm the efficiency of gene therapy DNA vector VTvaf17-HGF carrying the HGF therapeutic gene, where
1—cDNA of HGF gene before transfection with gene therapy DNA vector VTvaf17-HGF,
2—cDNA of HGF gene after transfection with gene therapy DNA vector VTvaf17-HGF,
3—cDNA of B2M gene before transfection with gene therapy DNA vector VTvaf17-HGF,
4—cDNA of B2M gene in cells after transfection with gene therapy DNA vector VTvaf17-HGF.
B2M (beta-2-microglobuline) gene listed in the GenBank database under number NM 004048.2 was used as a reference gene.

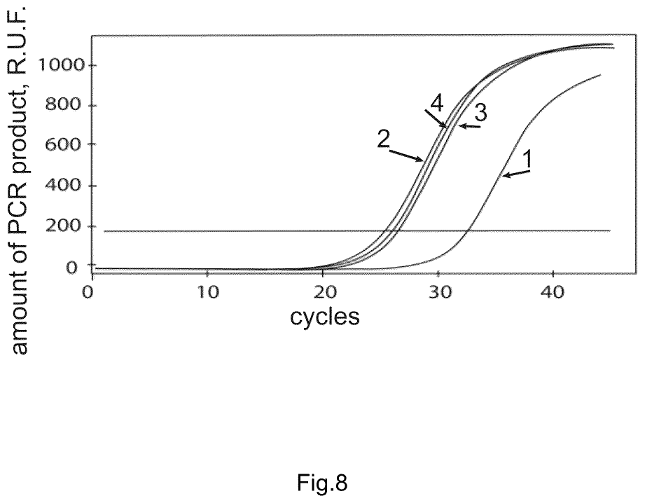

FIG. 8
shows diagrams of mRNA accumulation of the therapeutic gene, namely the human SDF1 gene, in HEKa human epidermal keratinocyte culture cells before their transfection and 48 hours after transfection of these cells with the DNA vector VTvaf17-SDF1 in order to confirm the efficiency of gene therapy DNA vector VTvaf17-SDF1 carrying the SDF1 therapeutic gene, where
1—cDNA of SDF1 gene before transfection with gene therapy DNA vector VTvaf17-SDF1,
2—cDNA of SDF1 gene before transfection with gene therapy DNA vector VTvaf17-SDF1,
3—cDNA of B2M gene before transfection with gene therapy DNA vector VTvaf17-SDF1,
4—cDNA of B2M gene in cells after transfection with gene therapy DNA vector VTvaf17-SDF1.
B2M (beta-2-microglobuline) gene listed in the GenBank database under number NM 004048.2 was used as a reference gene.

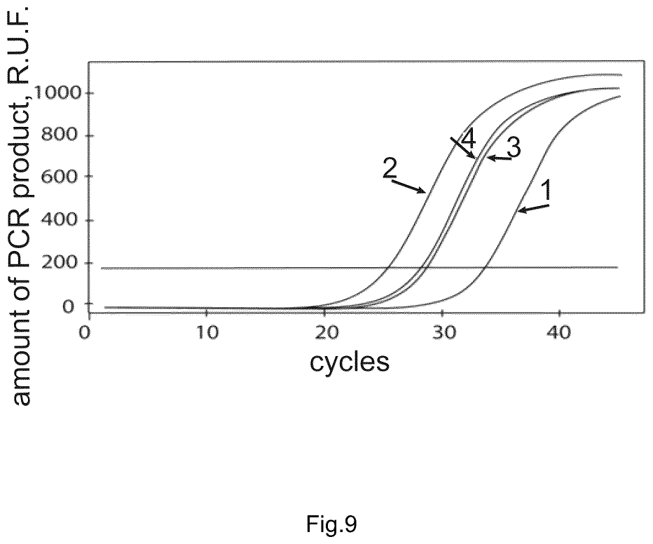

FIG. 9
shows diagrams of mRNA accumulation of the therapeutic gene, namely the human KLK4 gene, in HUVEC human umbilical vein endothelial culture cells before their transfection and 48 hours after transfection of these cells with the DNA vector VTvaf17-KLK4 in order to confirm the efficiency of gene therapy DNA vector VTvaf17-KLK4 carrying the KLK4 therapeutic gene, where
1—cDNA of KLK4 gene before transfection with gene therapy DNA vector VTvaf17-KLK4,
2—cDNA of KLK4 gene after transfection with gene therapy DNA vector VTvaf17-KLK4,
3—cDNA of B2M gene before transfection with gene therapy DNA vector VTvaf17-KLK4,
4—cDNA of B2M gene in cells after transfection with gene therapy DNA vector VTvaf17-KLK4.
B2M (beta-2-microglobuline) gene listed in the GenBank database under number NM 004048.2 was used as a reference gene.

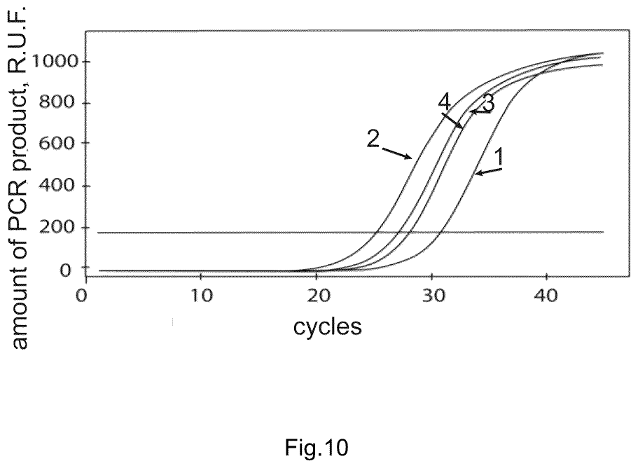

FIG. 10
shows diagrams of mRNA accumulation of the therapeutic gene, namely the human PDGFC gene, in HEMa human epidermal melanocyte culture cells before their transfection and 48 hours after transfection of these cells with the DNA vector VTvaf17-PDGFC in order to confirm the efficiency of gene therapy DNA vector VTvaf17-PDGFC carrying the PDGFC therapeutic gene, where
1—cDNA of PDGFC gene before transfection with gene therapy DNA vector VTvaf17-PDGFC,
2—cDNA of PDGFC gene after transfection with gene therapy DNA vector VTvaf17-PDGFC,
3—cDNA of B2M gene before transfection with gene therapy DNA vector VTvaf17-PDGFC,
4—cDNA of B2M gene in cells after transfection with gene therapy DNA vector VTvaf17-PDGFC.
B2M (beta-2-microglobuline) gene listed in the GenBank database under number NM 004048.2 was used as a reference gene.

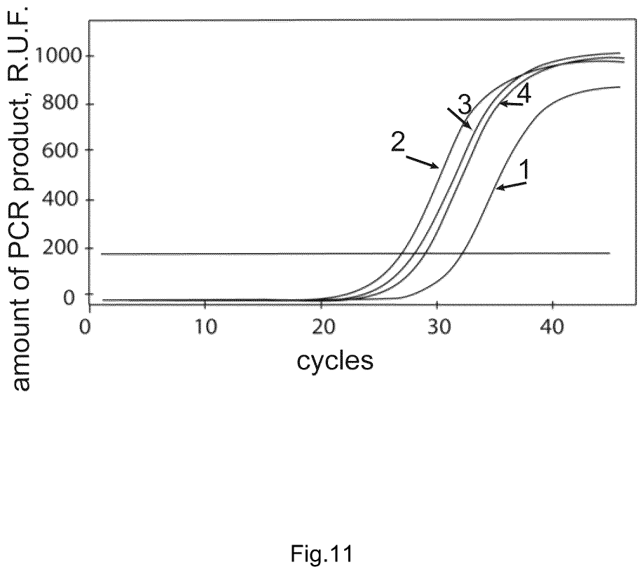

FIG. 11
shows diagrams of mRNA accumulation of the therapeutic gene, namely the human PROK1 gene, in HSkM human skeletal muscle myoblast culture cells before their transfection and 48 hours after transfection of these cells with the DNA vector VTvaf17-PROK1 in order to confirm the efficiency of gene therapy DNA vector VTvaf17-PROK1 carrying the PROK1 therapeutic gene, where
1—cDNA of PROK1 gene before transfection with gene therapy DNA vector VTvaf17-PROK1,
2—cDNA of PPROK1 gene after transfection with gene therapy DNA vector VTvaf17-PROK1,
3—cDNA of B2M gene before transfection with gene therapy DNA vector VTvaf17-PROK1,
4—cDNA of B2M gene in cells after transfection with gene therapy DNA vector VTvaf17-PROK1.
B2M (beta-2-microglobuline) gene listed in the GenBank database under number NM 004048.2 was used as a reference gene.

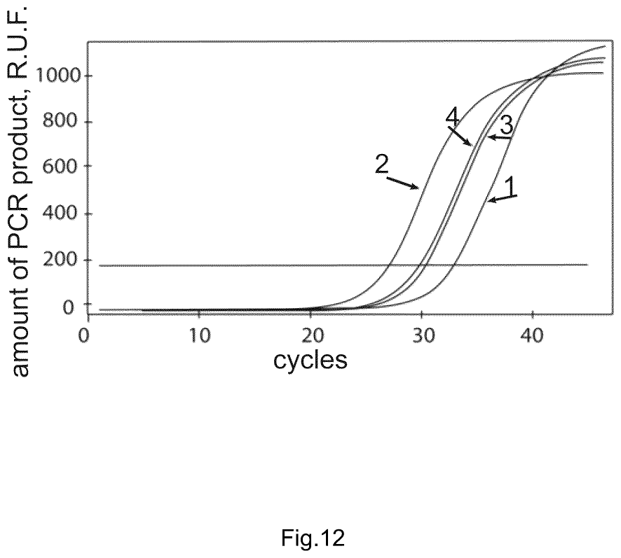

FIG. 12
shows diagrams of mRNA accumulation of the therapeutic gene, namely the human PROK2 gene in HMEC-1 human primary dermal microvascular endothelial cells before their transfection and 48 hours after transfection of these cells with the DNA vector VTvaf17-PROK2 in order to confirm the efficiency of gene therapy DNA vector VTvaf17-PROK2 carrying the PROK2 therapeutic gene, where
1—cDNA of PROK2 gene before transfection with gene therapy DNA vector VTvaf17-PROK2,
2—cDNA of PROK2 gene after transfection with gene therapy DNA vector VTvaf17-PROK2,
3—cDNA of B2M gene before transfection with gene therapy DNA vector VTvaf17-PROK2,
4—cDNA of B2M gene in cells after transfection with gene therapy DNA vector VTvaf17-PROK2.
B2M (beta-2-microglobuline) gene listed in the GenBank database under number NM 004048.2 was used as a reference gene.

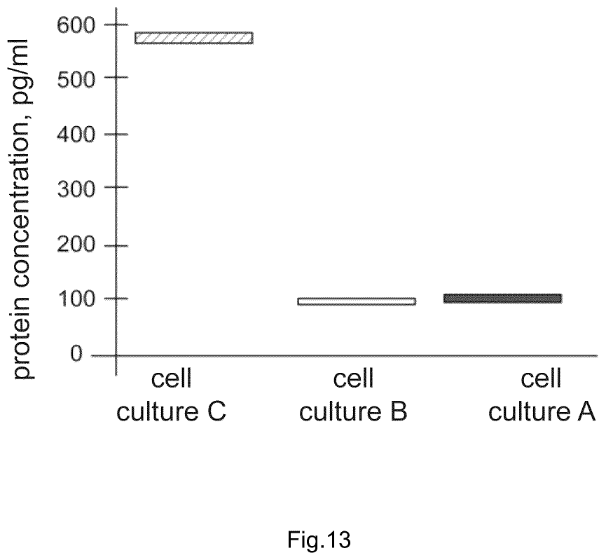

FIG. 13
shows the plot of angiogenin concentration in the culture medium of HDFa human dermal fibroblast cells (ATCC PCS-201-012) after transfection of these cells with the DNA vector VTvaf17-ANG in order to assess changes in angiogenin concentration in the culture medium of HDFa human dermal fibroblast cells (ATCC PCS-201-012) upon transfection of these cells with DNA vector VTvaf17-ANG carrying the ANG gene, where
culture A—HDFa human primary dermal fibroblast cells transfected with aqueous solution without plasmid DNA (reference),
culture B—HDFa human primary dermal fibroblast cells transfected with DNA vector VTvaf17,
culture C—HDFa human primary dermal fibroblast cells transfected with DNA vector VTvaf17-ANG carrying the ANG gene.

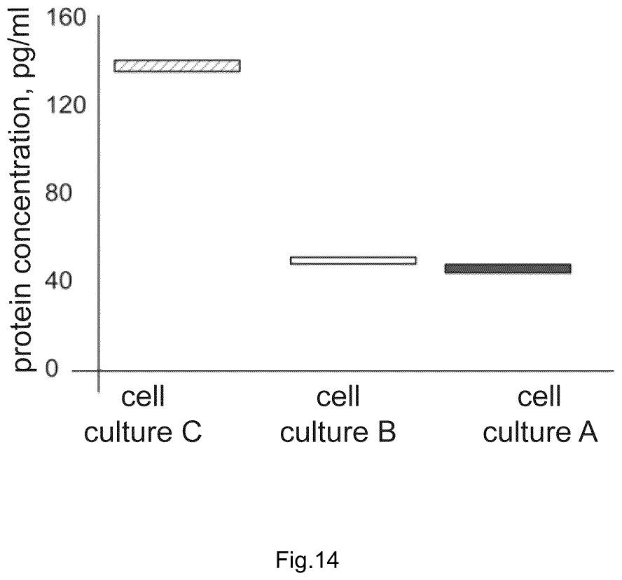

FIG. 14
shows the plot of angiopoietin 1 concentration in the culture medium of HT 297.T human fibroblast culture cells after transfection of these cells with the DNA vector VTvaf17-ANGPT1 in order to assess changes in the angiopoietin 1 concentration in the culture medium of HT 297.T human fibroblast culture cells upon transfection of these cells with DNA vector VTvaf17-ANGPT1 carrying the ANGPT1 gene, where
culture A—HT 297.T human fibroblast culture transfected with aqueous solution without plasmid DNA (reference),
culture B—HT 297.T human fibroblast culture transfected with DNA vector VTvaf17,
culture C—HT 297.T human fibroblast culture transfected with DNA vector VTvaf17-ANGPT1 carrying the ANGPT1 gene.

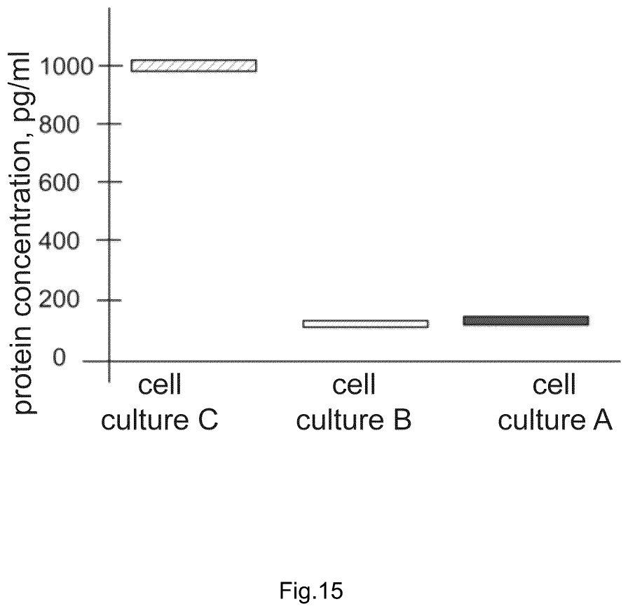

FIG. 15
shows the plot of vascular endothelial growth factor protein A concentration in the culture medium of Hs27 human foreskin fibroblasts after transfection of these cells with the DNA vector VTvaf17-VEGFA in order to assess changes in the vascular endothelial growth factor protein A concentration in the culture medium of Hs27 human foreskin fibroblasts upon transfection of these cells with DNA vector Tvaf17-VEGFA carrying the VEGFA gene, where
culture A—Hs27 human foreskin fibroblast culture transfected with aqueous solution without plasmid DNA (reference),
culture B—Hs27 human foreskin fibroblast culture transfected with DNA vector VTvaf17,
culture C—Hs27 human foreskin fibroblast culture transfected with DNA vector VTvaf17-VEGFA carrying the VEGFA gene.

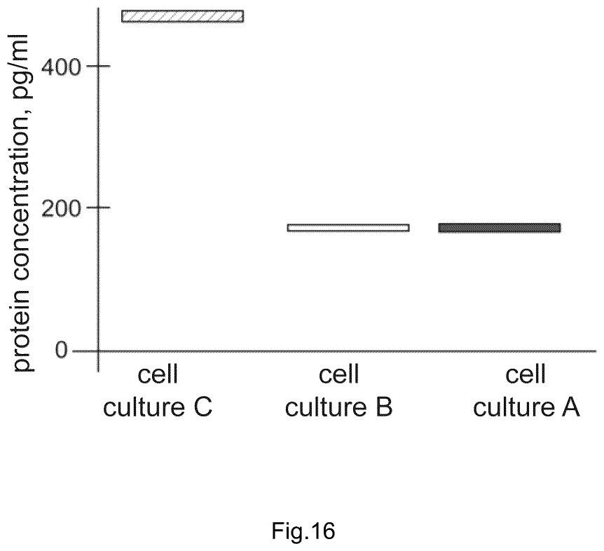

FIG. 16
shows the plot of fibroblast growth factor 1 protein concentration in the culture medium of HSkM human skeletal muscle myoblasts after transfection of these cells with the DNA vector VTvaf17-FGF1 in order to assess changes in the fibroblast growth factor 1 protein concentration in the culture medium of HSkM human skeletal muscle myoblasts upon transfection of these cells with DNA vector VTvaf17-FGF1 carrying the FGF1 gene, where
culture A—HSkM human skeletal muscle myoblast culture transfected with aqueous solution without plasmid DNA (reference),
culture B—HSkM human skeletal muscle myoblast culture transfected with DNA vector VTvaf17,
culture C—HSkM human skeletal muscle myoblast culture transfected with DNA vector VTvaf17-FGF1 carrying the FGF1 gene.

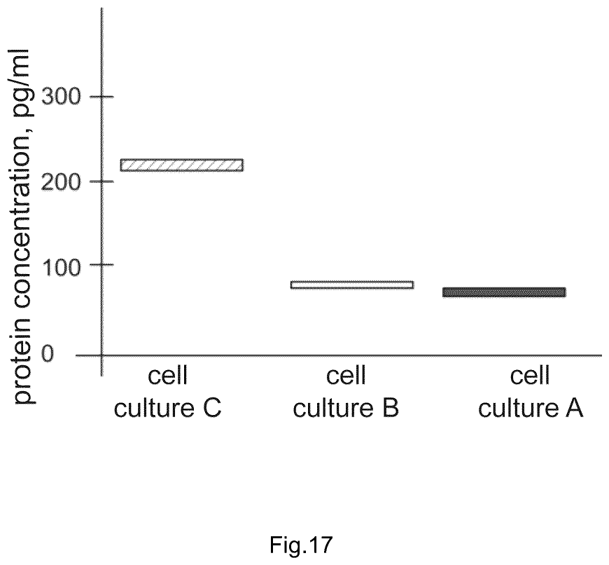

FIG. 17
shows the plot of hypoxia-inducible factor 1α protein concentration in the culture medium of HBdSMc human urinary bladder smooth muscle cells after transfection of these cells with DNA vector VTvaf17-HIF1α in order to assess changes in the hypoxia-inducible factor 1α protein concentration in the culture medium of HBdSMc human urinary bladder smooth muscle cells upon transfection of these cells with DNA vector VTvaf17-HIF1α carrying the HIF1α gene, where
culture A—HBdSMc human urinary bladder smooth muscle cell culture transfected with aqueous solution without plasmid DNA (reference),
culture B—HBdSMc human urinary bladder smooth muscle cell culture transfected with DNA vector VTvaf17,
culture C—HBdSMc human urinary bladder smooth muscle cell culture transfected with DNA vector VTvaf17-HIF1α carrying the HIF1α gene.

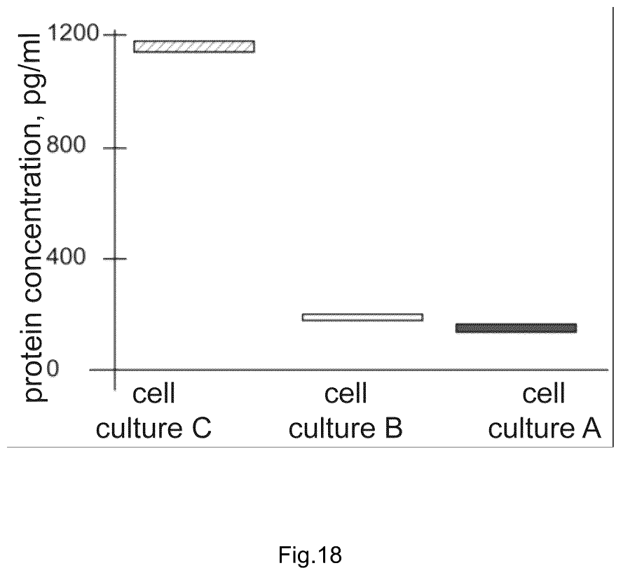

FIG. 18
shows the plot of hepatocyte growth factor protein concentration in the culture medium of T/GHA VSMC human aortic smooth muscle cells after transfection of these cells with DNA vector VTvaf17-HGF in order to assess changes in the hepatocyte growth factor protein concentration in the culture medium of T/GHA VSMC human aortic smooth muscle cells upon transfection of these cells with DNA vector VTvaf17-HGF carrying the HGF gene, where
culture A—T/GHA VSMC human aortic smooth muscle cell culture transfected with aqueous solution without plasmid DNA (reference),
culture B—T/GHA VSMC human aortic smooth muscle cell culture transfected with DNA vector VTvaf17,
culture C—T/GHA VSMC human aortic smooth muscle cell culture transfected with DNA vector VTvaf17-HGF carrying the HGF gene.

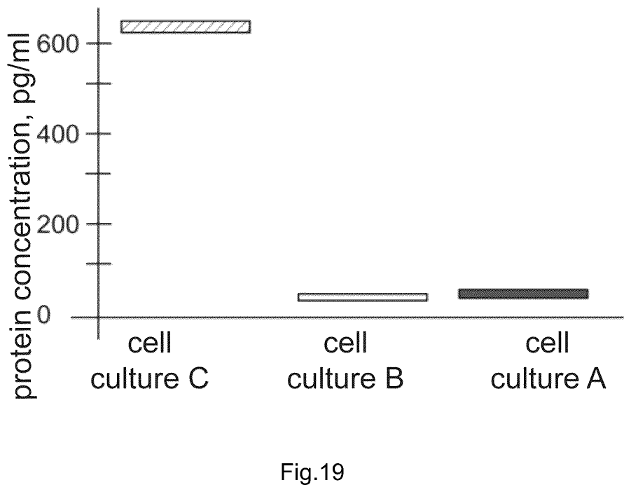

FIG. 19
shows the plot of stromal cell-derived factor 1 protein concentration in the culture medium of HEKa human epidermal keratinocytes after transfection of these cells with the DNA vector VTvaf17-SDF1 in order to assess changes in the stromal cell-derived factor 1 protein concentration in the culture medium of HEKa human epidermal keratinocytes upon transfection of these cells with DNA vector VTvaf17-SDF1 carrying the SDF1 gene, where
culture A—HEKa human epidermal keratinocyte culture transfected with aqueous solution without plasmid DNA (reference),
culture B—HEKa human epidermal keratinocyte culture transfected with DNA vector VTvaf17,
culture B—HEKa human epidermal keratinocyte culture transfected with DNA vector VTvaf17-SDF1 carrying the SDF1 gene.

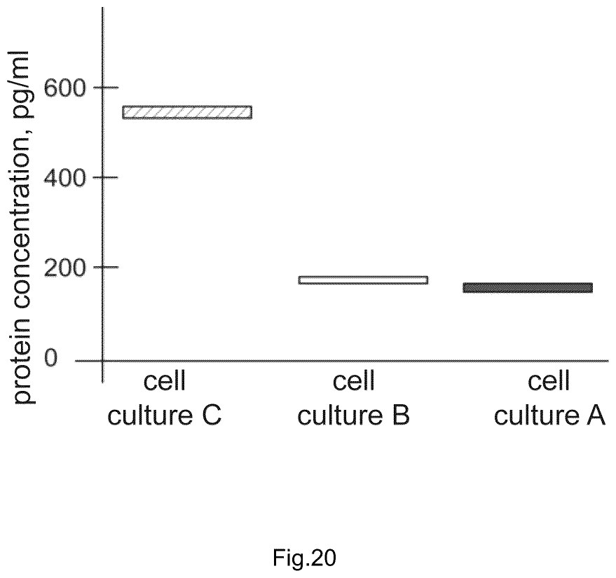

FIG. 20
shows the plot of kallikrein-like protein concentration in the culture medium of HUVEC human umbilical vein endothelial cells after transfection of these cells with the DNA vector VTvaf17-KLK4 in order to assess changes in the kallikrein-like protein concentration in the culture medium of HUVEC human umbilical vein endothelial cells upon transfection of these cells with DNA vector VTvaf17-KLK4 carrying the KLK4 gene, where
culture A—HUVEC human umbilical vein endothelial culture transfected with aqueous solution without plasmid DNA (reference),
culture B—HUVEC human umbilical vein endothelial culture transfected with DNA vector VTvaf17,
culture C—HUVEC human umbilical vein endothelial culture transfected with DNA vector VTvaf17-KLK4 carrying the KLK4 gene.

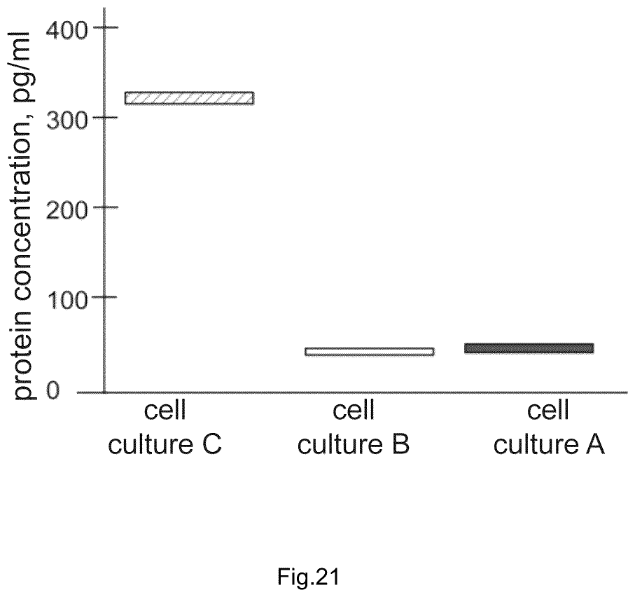

FIG. 21
shows the plot of platelet growth factor C protein concentration in the culture medium of HEMa human epidermal melanocytes after transfection of these cells with the DNA vector VTvaf17-PDGFC in order to assess changes in the platelet growth factor C protein concentration in the culture medium of HEMa human epidermal melanocytes upon transfection of these cells with DNA vector VTvaf17-PDGFC carrying the PDGFC gene, where
culture A—HEMa human epidermal melanocyte culture transfected with aqueous solution without plasmid DNA (reference),
culture B—HEMa human epidermal melanocyte culture transfected with DNA vector VTvaf17,
culture C—HEMa human epidermal melanocyte culture transfected with DNA vector VTvaf17-PDGFC carrying the PDGFC gene.

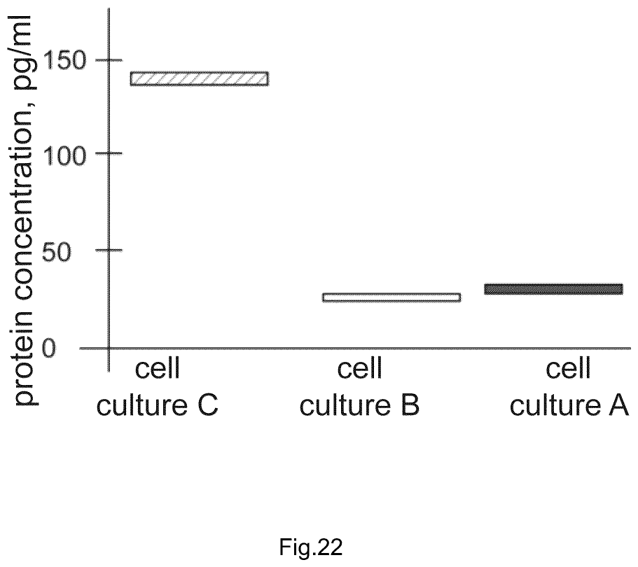

FIG. 22
shows the plot of prokineticin-1 protein concentration in the culture medium of HSkM human skeletal muscle myoblasts after transfection of these cells with the DNA vector VTvaf17-PROK1 in order to assess changes in the prokineticin-1 protein concentration in the culture medium of HSkM human skeletal muscle myoblasts upon transfection of these cells with DNA vector VTvaf17-PROK1 carrying the PROK1 gene, where
culture A—HSkM human skeletal muscle myoblast culture transfected with aqueous solution without plasmid DNA (reference),
culture B—HSkM human skeletal muscle myoblast culture transfected with DNA vector VTvaf17,
culture C—HSkM human skeletal muscle myoblast culture transfected with DNA vector VTvaf17-PROK1 carrying the PROK1 gene.

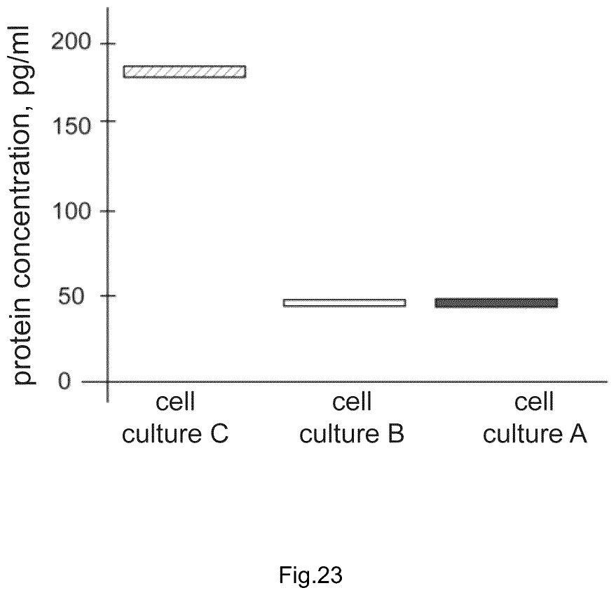

FIG. 23
shows the plot of prokineticin-2 protein concentration in the culture medium of HMEC-1 human dermal microvascular endothelial cells after transfection of these cells with the DNA vector VTvaf17-PROK2 in order to assess changes in the prokineticin-2 protein concentration in the culture medium of HMEC-1 human dermal microvascular endothelial cells upon transfection of these cells with DNA vector VTvaf17-PROK2 carrying the PROK2 gene, where
culture A—HMEC-1 human dermal microvascular endothelial cells transfected with aqueous solution without plasmid DNA (reference),
culture B—HMEC-1 human dermal microvascular endothelial cells transfected with DNA vector VTvaf17,
culture B—HMEC-1 human dermal microvascular endothelial cells transfected with DNA vector VTvaf17-PROK2 carrying the PROK2 gene.

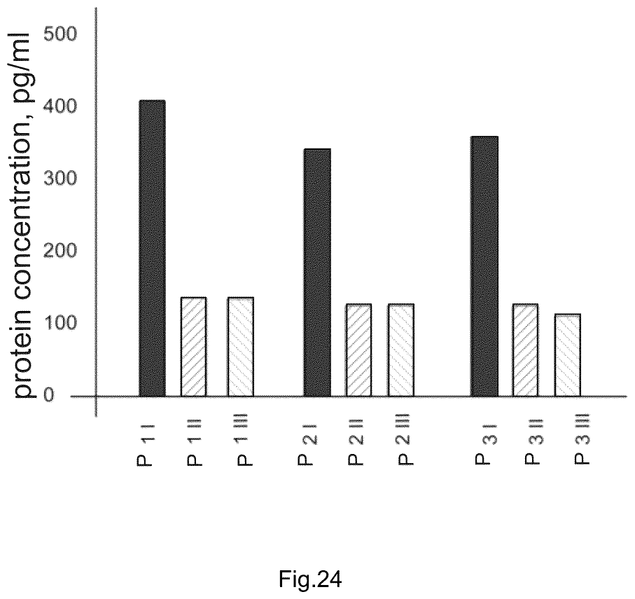

FIG. 24
shows the plot of ANG angiogenin concentration in the skin biopsy specimens of three patients after injection of gene therapy DNA vector VTvaf17-ANG into the skin of these patients in order to assess the functional activity, i.e. the expression of the therapeutic gene at the protein level, and the possibility of increasing the level of angiogenin expression using gene therapy DNA vector based on gene therapy vector VTvaf17 carrying the ANG therapeutic gene.
The following elements are indicated in FIG. 24:
P1I—patient P1 skin biopsy in the region of injection of gene therapy DNA vector VTvaf17-ANG,
P1II—patient P1 skin biopsy in the region of injection of gene therapy DNA vector VTvaf17 (placebo),
P1III—patient P1 skin biopsy from intact site,
P2I—patient P2 skin biopsy in the region of injection of gene therapy DNA vector VTvaf17-ANG,
P2II—patient P2 skin biopsy in the region of injection of gene therapy DNA vector VTvaf17 (placebo),
P2III—patient P2 skin biopsy from intact site,
P3I—patient P3 skin biopsy in the region of injection of gene therapy DNA vector VTvaf17-ANG,
P3II—patient P3 skin biopsy in the region of injection of gene therapy DNA vector VTvaf17 (placebo),
P3III—patient P3 skin biopsy from intact site.

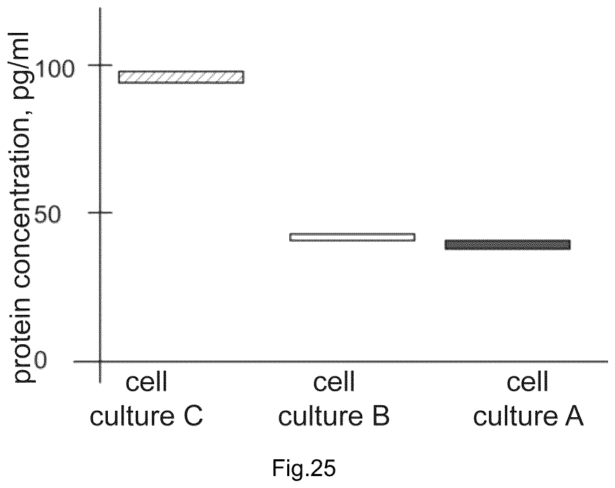

FIG. 25
shows the plot of angiopoietin ANGPT1 concentration in human skin biopsy samples after injection of autologous fibroblast culture into the skin transfected with the gene therapy DNA vector VTvaf17-ANGPT1 in order to demonstrate the method of usage by introducing autologous cells transfected with the gene therapy DNA vector VTvaf17-ANGPT1.
The following elements are indicated in FIG. 25:
P1A—patient P1 skin biopsy from intact site,
P1B—patient P1 skin biopsy in the region of injection of autologous fibroblasts of the patient transfected with gene therapy DNA vector VTvaf17,
P1C—patient P1 skin biopsy in the region of injection of autologous fibroblast culture of the patient transfected with gene therapy DNA vector VTvaf17-ANGPT1.

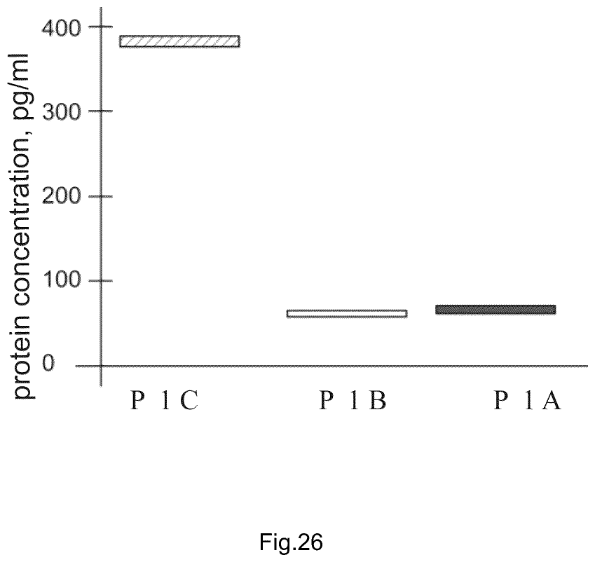

FIG. 26
shows the plot of SDF1 stromal cell-derived factor protein concentration in biopsy samples of patient's muscle tissue after injection of gene therapy DNA vector VTvaf17-SDF1 into the patient's muscle tissue in order to assess the functional activity, i.e. the expression of the therapeutic gene at the protein level, and the possibility of increasing the level of protein expression using gene therapy DNA vector based on gene therapy vector VTvaf17 carrying the SDF1 therapeutic gene.
The following elements are indicated in FIG. 26:
P1I—patient P1 muscle biopsy in the region of injection of gene therapy DNA vector VTvaf17-SDF1,
P1II—patient P1 muscle biopsy in the region of injection of gene therapy DNA vector VTvaf17 (placebo),
P1III—patient P1 muscle biopsy from intact site.

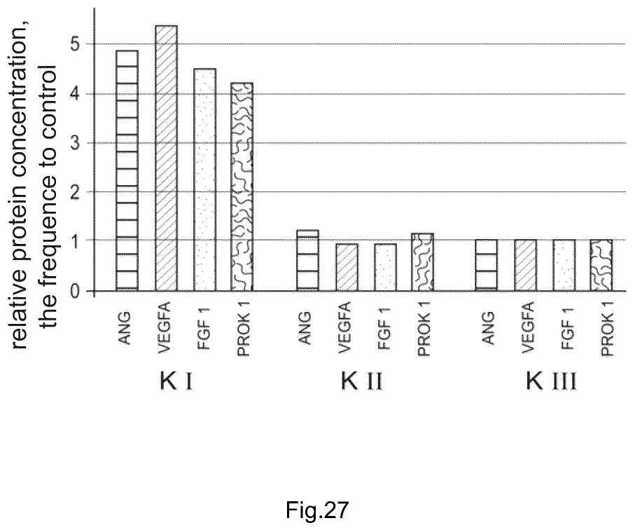

FIG. 27
shows the diagram of change in the protein concentrations: angiogenin (ANG), vascular endothelial growth factor A (VEGFA), fibroblast growth factor 1 (FGF1), and prokineticin-1 (PROK1) in rat skin biopsy samples in the injection site:
in group 1 (KI)—a mixture of gene therapy DNA vectors VTvaf17-ANG, VTvaf17-VEGFA, VTvaf17-FGF1, VTvaf17-PROK1,
in group 2 (KII)—DNA vector VTvaf17 solution (placebo),
in group 3 (KIII)—a saline solution.

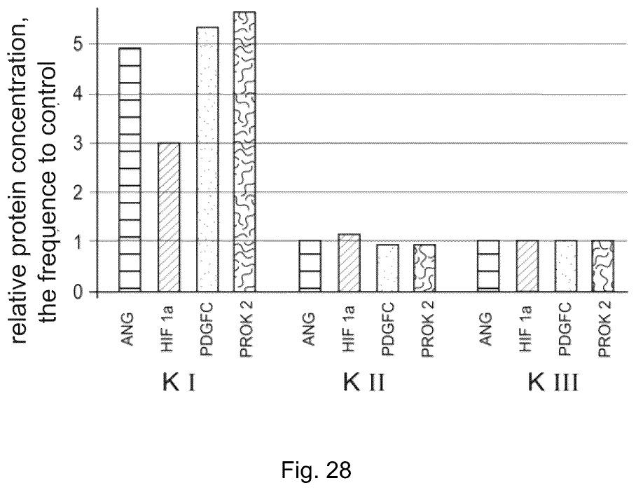

FIG. 28
shows the diagram of change in the protein concentrations: angiogenin (ANG), hypoxia-inducible factor (HIF1α), platelet growth factor C (PDGFC), prokineticin-2 (PROK2) in muscle biopsy samples in the patient's forearm area after injection:
P1I—a mixture of gene therapy DNA vectors VTvaf17-ANG, VTvaf17-HIF1α, VTvaf17-PDGFC, VTvaf17-PROK2,
P1II—gene therapy DNA vector VTvaf17 solution (placebo),
P1III—intact site.

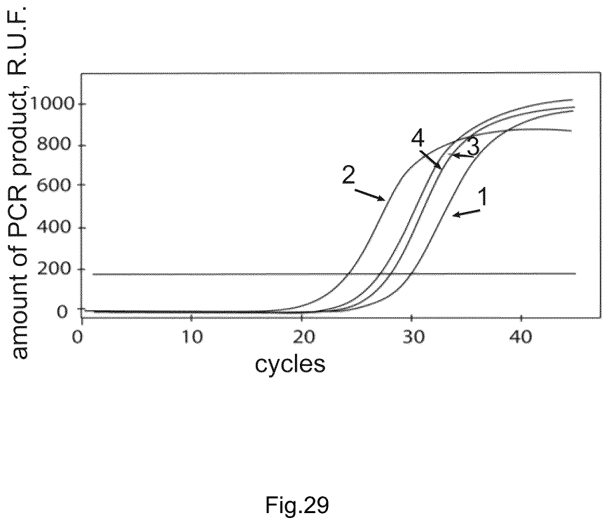

FIG. 29
shows diagrams of cDNA amplicon accumulation of the HGF therapeutic gene in BAOSMC bovine aortic smooth muscle cells (Genlantis) before and 48 hours after transfection of these cells with the DNA vector VTvaf17-HGF in order to demonstrate the method of use by introducing the gene therapy DNA vector in animal cages.
Curves of accumulation of amplicons during the reaction are shown in FIG. 29 corresponding to:
1—cDNA of HGF gene in BAOSMC bovine aortic smooth muscle cells before transfection with gene therapy DNA vectorVTvaf17-HGF,
2—cDNA of HGF gene in BAOSMC bovine aortic smooth muscle cells after transfection with gene therapy DNA vectorVTvaf17-HGF,
3—cDNA of ACT gene in BAOSMC bovine aortic smooth muscle cells before transfection with gene therapy DNA vectorVTvaf17-HGF,
4—cDNA of ACT gene in BAOSMC bovine aortic smooth muscle cells after transfection with gene therapy DNA vectorVTvaf17-HGF.
Bull/cow actin gene (ACT) listed in the GenBank database under number AH001130.2 was used as a reference gene.

### EMBODIMENT OF THE INVENTION

Gene therapy DNA vectors carrying the therapeutic human genes—ANG gene encoding the angiogenin, ANGPT1 gene encoding the angiopoietin 1, VEGFA gene encoding the vascular endothelial growth factor A, FGF1 gene encoding fibroblast growth factor 1, HIF1α gene encoding hypoxia inducible factor-α, HGF gene encoding hepatocyte growth factor, gene SDF1 encoding stromal cell-derived factor, KLK4 gene encoding the kallikrein-like, PDGFC gene encoding platelet growth factor C, PROK1 gene encoding prokineticin 1, PROK2 gene encoding prokineticin 2 designed to increase the level of expression of these therapeutic genes in human and animal tissues were constructed based on 3165 bp gene therapy DNA vector VTvaf17. The method of production of each gene therapy DNA vector carrying human therapeutic genes involves cloning of the protein coding sequence of the therapeutic gene to the polylinker of the gene therapy DNA vector VTvaf17 selected from the group of the following genes: ANG, ANGPT1, VEGFA, FGF1, HIF1α, HGF, SDF1, KLK4, PDGFC, PROK1, and PROK2.

The method of production of gene therapy DNA vector based on gene therapy DNA vector VTvaf17 carrying the therapeutic gene selected from the group of ANG, ANGPT1, VEGFA, FGF1, HIF1α, HGF, SDF1, KLK4, PDGFC, PROK1, and PROK2 genes involves

1. obtaining a 448 bp long coding region of the ANG gene, or a 1501 bp long coding region of the ANGPT1 gene, or a 1242 bp long coding region of the VEGFA gene, or a 472 bp long coding region of the FGF1 gene, or a 2485 bp long coding region of the HIF1α gene, or a 2190 bp long coding region of the HGF gene, or a 284 bp long coding region of the SDF1 gene, or a 769 bp long coding region of the KLK4 gene, or a 1041 bp long coding region of the PDGFC gene, or a 328 bp long coding region of the PROK1 gene, or a 394 bp long coding region of the PROK2 gene by extracting total RNA from the normal biological human tissue sample, followed by a reverse transcription reaction and PCR amplification using oligonucleotides constructed for this purpose by chemical synthesis, followed by the cleavage of amplification product by SalI and KpnI, or BamHI and HindIII, or BamHI and SalI, or BamHI and EcoRI, or SalI and EcoRI restriction endonucleases.

2. The coding region of the ANG therapeutic gene, or ANGPT1 therapeutic gene, or FGF1 therapeutic gene, or HIF1α therapeutic gene, or PDGFC therapeutic gene, or PROK2 therapeutic gene was cloned to the polylinker of gene therapy DNA vector VTvaf17 by SalI-KpnI sites, the coding region of the VEGFA therapeutic gene was cloned to the polylinker of gene therapy DNA vector VTvaf17 by BamHI-HindIII sites, the coding region of the HGF therapeutic gene was cloned to the polylinker of gene therapy DNA vector VTvaf17 by BamHI-SalI sites, the coding region of the SDF1 therapeutic gene was cloned to the polylinker of gene therapy DNA vector VTvaf17 by BamHI-EcoRI sites, the coding region of the KLK4 or PROK1 therapeutic gene was cloned to the polylinker of gene therapy DNA vector VTvaf17 by SalI-EcoRI sites, and, as a result, gene therapy DNA vector VTvaf17-ANG, SEQ ID No. 1, or VTvaf17-ANGPT1, SEQ ID No. 2, or VTvaf17-VEGFA, SEQ ID No. 3, or VTvaf17-FGF1, SEQ ID No. 4, or VTvaf17-HIF1α, SEQ ID No. 5, or VTvaf17-HGF, SEQ ID No. 6, or VTvaf17-SDF1, SEQ ID No. 7, or VTvaf17-KLK4, SEQ ID No. 8, or VTvaf17-PDGFC, SEQ ID No. 9, or VTvaf17-PROK1, SEQ ID No. 10, or VTvaf17-PROK2, SEQ ID No. 11 was produced. The obtained gene therapy DNA vector VTvaf17 carrying the therapeutic gene selected from the group of ANG, ANGPT1, VEGFA, FGF1, HIF1α, HGF, SDF1, KLK4, PDGFC, PROK1, and PROK2 genes was transformed by electroporation of Escherichia coli strain SCS110-AF with antibiotic-free selection of the obtained clones.

3. in order to confirm the efficiency of the constructed gene therapy DNA vector VTvaf17-ANG, SEQ ID No. 1, or VTvaf17-ANGPT1, SEQ ID No. 2, or VTvaf17-VEGFA, SEQ ID No. 3, or VTvaf17-FGF1, SEQ ID No. 4, or VTvaf17-HIF1α, SEQ ID No. 5, or VTvaf17-HGF, SEQ ID No. 6, or VTvaf17-SDF1, SEQ ID No. 7, or VTvaf17-KLK4, SEQ ID No. 8, or VTvaf17-PDGFC, SEQ ID No. 9, or VTvaf17-PROK1, SEQ ID No. 10, or VTvaf17-PROK2, SEQ ID No. 11, the following was assessed:
A) change in mRNA accumulation of therapeutic genes in the human cells after transfection of different cell lines with gene therapy DNA vectors (by real-time PCR-RT-PCR),
B) change in the quantitative level of therapeutic proteins in the human cell culture medium after transfection of different cell lines with gene therapy DNA vectors (using enzyme-linked immunosorbent assay ELISA),
C) change in the quantitative level of therapeutic proteins in the supernatant of human and animals tissue biopsy specimens after the injection of gene therapy DNA vectors into these tissues (using ELISA),
D) change in the quantitative level of therapeutic proteins in the supernatant of human tissue biopsies after the injection of these tissues with autologous cells of this human transfected with gene therapy DNA vectors (using ELISA),

In order to confirm the practicability of use of the constructed gene therapy DNA vector VTvaf17-ANG, SEQ ID No. 1, or VTvaf17-ANGPT1, SEQ ID No. 2, or VTvaf17-VEGFA, SEQ ID No. 3, or VTvaf17-FGF1, SEQ ID No. 4, or VTvaf17-HIF1α, SEQ ID No. 5, or VTvaf17-HGF, SEQ ID No. 6, or VTvaf17-SDF1, SEQ ID No. 7, or VTvaf17-KLK4, SEQ ID No. 8, or VTvaf17-PDGFC, SEQ ID No. 9, or VTvaf17-PROK1, SEQ ID No. 10, or VTvaf17-PROK2, SEQ ID No. 11, the following was performed:
A) transfection with gene therapy DNA vectors of different human and animal cell lines,
B) injection of gene therapy DNA vectors into different human and animal tissues,
C) injection of gene therapy DNA vectors into human and animal tissues;
D) injection of autologous cells transfected with gene therapy DNA vectors into human tissues.

In order to confirm the construction of Escherichia coli strain SCS110-AF/VTvaf17-ANG, or Escherichia coli strain SCS110-AF/VTvaf17-ANGPT1, or Escherichia coli strain SCS110-AF/VTvaf17-VEGFA, or Escherichia coli strain SCS110-AF/VTvaf17-FGF1, or Escherichia coli strain SCS110-AF/VTvaf17-HIF1α, or Escherichia coli strain SCS110-AF/VTvaf17-HGF, or Escherichia coli strain SCS110-AF/VTvaf17-SDF1, or Escherichia coli strain SCS110-AF/VTvaf17-KLK4, or Escherichia coli strain SCS110-AF/VTvaf17-PDGFC, or Escherichia coli strain SCS110-AF/VTvaf17-PROK1, or Escherichia coli strain SCS110-AF/VTvaf17-PROK2 transformation, selection, and subsequent biomass growth with extraction of plasmid DNA were performed.

To confirm the producibility and constructability on an industrial scale of gene therapy DNA vector VTvaf17-ANG, SEQ ID No. 1, or VTvaf17-ANGPT1, SEQ ID No. 2, or VTvaf17-VEGFA, SEQ ID No. 3, or VTvaf17-FGF1, SEQ ID No. 4, or VTvaf17-HIF1α, SEQ ID No. 5, or VTvaf17-HGF, SEQ ID No. 6, or VTvaf17-SDF1, SEQ ID No. 7, or VTvaf17-KLK4, SEQ ID No. 8, or VTvaf17-PDGFC, SEQ ID No. 9 or VTvaf17-PROK1, SEQ ID No. 10, or VTvaf17-PROK2, SEQ ID No. 11, the following was performed:
A) fermentation on an industrial scale of Escherichia coli strain SCS110-AF/VTvaf17-ANG, or Escherichia coli strain SCS110-AF/VTvaf17-ANGPT1, or Escherichia coli strain SCS110-AF/VTvaf17-VEGFA, or Escherichia coli strain SCS110-AF/VTvaf17-FGF1, or Escherichia coli strain SCS110-AF/VTvaf17-HIF1α, or Escherichia coli strain SCS110-AF/VTvaf17-HGF, or Escherichia coli strain SCS110-AF/VTvaf17-SDF1, or Escherichia coli strain SCS110-AF/VTvaf17-KLK4, or Escherichia coli strain SCS110-AF/VTvaf17-PDGFC, or Escherichia coli strain SCS110-AF/VTvaf17-PROK1, or Escherichia coli strain SCS110-AF/VTvaf17-PROK2, each containing gene therapy DNA vector VTvaf17 carrying a protein-coding sequence of the therapeutic gene selected from the group of ANG, ANGPT1, VEGFA, FGF1, HIF1α, HGF, SDF1, KLK4, PDGFC, PROK1, and PROK2 genes.

### Example 1

### Production of Gene Therapy DNA Vector VTvaf17-ANG Carrying the ANG Therapeutic Gene.

Gene therapy DNA vector VTvaf17-ANG was constructed by cloning the coding region of the ANG gene to the DNA vector VTvaf17 by SalI-KpnI restriction sites. The coding region of ANG gene (448 bp) was obtained by isolating total RNA from the biological human tissue sample followed by reverse transcription reaction using commercial kit Mint-2 (Evrogen, Russia) and constructed oligonucleotides

ANG_F
TTTGTCGACCACCATGGTGATGGGCCTGGGCGTT,
ANG_R
AATGGTACCTTACGGACGACGGAAAATTGACTG

and PCR amplification using the commercially available kit Phusion® High-Fidelity DNA Polymerase (New England Biolabs, USA) and constructed oligonucleotides

The amplification product of the coding region of ANG gene and DNA vector VTvaf17 was cleaved by restriction endonucleases SalI and KpnI (New England Biolabs, USA).

This resulted in a 3618 bp DNA vector VTvaf17-ANG carrying the therapeutic gene, namely ANG gene, containing nucleotide sequence SEQ ID No. 1 allowing for antibiotic-free selection with the structure shown in FIG. 1.A.

Gene therapy DNA vector VTvaf17 was constructed by consolidating six fragments of DNA derived from different sources:
(a) the origin of replication (ori) was produced by PCR amplification of a region of commercially available plasmid pBR322 with a point mutation,
(b) EF1a promoter region was produced by PCR amplification of a site of human genomic DNA,
(c) hGH TA transcription terminator was produced by PCR amplification of a site of human genomic DNA,
(d) the RNA OUT regulatory site of transposon Tn10 was synthesised from oligonucleotides
(e) kanamycin resistance gene was produced by PCR amplification of a site of commercially available human plasmid pET-28,
(f) the polylinker was produced by annealing two synthetic oligonucleotides.

PCR amplification was performed using the commercially available kit Phusion® High-Fidelity DNA Polymerase (New England Biolabs) as per the manufacturer's instructions. The fragments have overlapping regions allowing for their consolidation with subsequent PCR amplification. Fragments (a) and (b) were consolidated using oligonucleotides Ori-F and EF1-R, and fragments (c), (d), and (e) were consolidated using oligonucleotides hGH-F and Kan-R. Afterwards, the produced fragments were consolidated by restriction with subsequent ligation by sites BamHI and NcoI. This resulted in a plasmid still devoid of the polylinker. To add it, the plasmid was cleaved by BamHI and EcoRI sites followed by ligation with fragment (f). Therefore, a 4182 bp vector was constructed carrying the kanamycin resistance gene flanked by SpeI restriction sites. Then this gene was cleaved by SpeI restriction sites and the remaining fragment was ligated to itself. This resulted in a 3165 bp gene therapy DNA vector VTvaf17 that is recombinant and allows for antibiotic-free selection.

### Example 2

### Production of Gene Therapy DNA Vector VTvaf17-ANGPT1 Carrying the ANGPT1 Therapeutic Gene.

Gene therapy DNA vector VTvaf17-ANGPT1 was constructed by cloning the coding region of the ANGPT1 gene to the DNA vector VTvaf17 by NheI and HindIII restriction sites. The coding region of ANGPT1 gene (1501 bp) was obtained by isolating total RNA from the biological human tissue sample followed by reverse transcription reaction using commercial kit Mint-2 (Evrogen, Russia) and constructed oligonucleotides

ANGPT1_F
TTTGTCGACCACCATGACAGTTTTCCTTTCCTTTGCTTTCC,
ANGPT1_R
AATGGTACCTCAAAAATCTAAAGGTCGAATCATCATAGTTG

and PCR amplification using the commercially available kit Phusion® High-Fidelity DNA Polymerase (New England Biolabs, USA) and constructed oligonucleotides.

The amplification product of the coding region of ANGPT1 gene and DNA vector VTvaf17 was cleaved by SalI and KpnI restriction endonucleases (New England Biolabs, USA).

This resulted in a 4660 bp DNA vector VTvaf17-ANGPT1 carrying the therapeutic gene, namely ANGPT1 gene, containing nucleotide sequence SEQ ID No. 2 allowing for antibiotic-free selection with the structure shown in FIG. 1.B.

Gene therapy DNA vector VTvaf17 was constructed as described in Example 1.

### Example 3

### Production of DNA Vector VTvaf17-VEGFA Carrying the Human VEGFA Therapeutic Gene.

Gene therapy DNA vector VTvaf17-VEGFA was constructed by cloning the coding region of the VEGFA gene to the DNA vector VTvaf17 by BamHI and HindIII restriction sites. The coding region of VEGFA gene (1242 bp) was obtained by isolating total RNA from the human tissue biopsy sample followed by reverse transcription reaction using commercial kit Mint-2 (Evrogen, Russia) and constructed oligonucleotides

VEGFA_F
GGGGGATCCACCATGACGGACAGACAGACAGACACCGC,
VEGFA_R
TTTGGATCCACCATGAACTTTCTGCTGTCTTGGGTGC

and PCR amplification using the commercially available kit Phusion® High-Fidelity DNA Polymerase (New England Biolabs, USA) and constructed oligonucleotides.

The amplification product of the coding region of VEGFA gene and DNA vector VTvaf17 was cleaved by BamHI and HindIII restriction endonucleases (New England Biolabs, USA).

This resulted in a 4395 bp gene therapy DNA vector VTvaf17-VEGFA containing nucleotide sequence SEQ ID No. 3 carrying the therapeutic gene, namely VEGFA, allowing for antibiotic-free selection with the structure shown in FIG. 1.C.

Gene therapy DNA vector VTvaf17 was constructed as described in Example 1.

### Example 4

### Production of Gene Therapy DNA Vector VTvaf17-FGF1 Carrying the FGF1 Therapeutic Gene.

Gene therapy DNA vector VTvaf17-FGF1 was constructed by cloning the coding region of the FGF1 gene to the DNA vector VTvaf17 by SalI-KpnI restriction sites. The coding region of FGF1 gene (472 bp) was obtained by isolating total RNA from the human tissue biopsy sample followed by reverse transcription reaction using commercial kit Mint-2 (Evrogen, Russia) and constructed oligonucleotides

FGF_F
TTTGTCGACCACCATGGCTGAAGGGGAAATCACC,
FGF_R
AATGGTACCTTAATCAGAAGAGACTGGCAGGGG

and PCR amplification using the commercially available kit Phusion® High-Fidelity DNA Polymerase (New England Biolabs, USA) and constructed oligonucleotides.

The amplification product of the coding region of FGF1 gene and DNA vector VTvaf17 was cleaved by restriction endonucleases SalI and KpnI (New England Biolabs, USA).

This resulted in a 3631 bp DNA vector VTvaf17-FGF1 carrying the therapeutic gene, namely FGF1 gene, containing nucleotide sequence SEQ ID No. 4 allowing for antibiotic-free selection with the structure shown in FIG. 1.D.

Gene therapy DNA vector VTvaf17 was constructed as described in Example 1.

### Example 5

### Production of Gene Therapy DNA Vector VTvaf17-HIF1α Carrying the HIF1α Therapeutic Gene.

Gene therapy DNA vector VTvaf17-HIF1α was constructed by cloning the coding region of the HIF1α gene to the DNA vector VTvaf17 by SalI-KpnI restriction sites. The coding region of HIF1α gene (2485 bp) was obtained by isolating total RNA from the human tissue biopsy sample followed by reverse transcription reaction using commercial kit Mint-2 (Evrogen, Russia) and constructed oligonucleotides

HIF_F
TTTGTCGACCACCATGGAGGGCGCCGGCGGCGCGA,
HIF_R
AATGGTACCTCAGTTAACTTGATCCAAAGCTCTGAGTAATTC

and PCR amplification using the commercially available kit Phusion® High-Fidelity DNA Polymerase (New England Biolabs, USA) and constructed oligonucleotides.

The amplification product of the coding region of HIF1α gene and DNA vector VTvaf17 was cleaved by SalI and KpnI restriction endonucleases (New England Biolabs, USA).

This resulted in a 5643 bp DNA vector VTvaf17-HIF1α carrying the therapeutic gene, namely HIF1α gene, containing nucleotide sequence SEQ ID No. 5 allowing for antibiotic-free selection with the structure shown in FIG. 1.E.

Gene therapy DNA vector VTvaf17 was constructed as described in Example 1.

### Example 6

### Production of Gene Therapy DNA Vector VTvaf17-HGF Carrying the HGF Therapeutic Gene.

Gene therapy DNA vector VTvaf17-HGF was constructed by cloning the coding region of HGF gene to the DNA vector VTvaf17 by BamHI-SalI restriction sites. The coding region of HGF gene (2190 bp) was obtained by isolating total RNA from the human tissue biopsy sample followed by reverse transcription reaction using commercial kit Mint-2 (Evrogen, Russia) and constructed oligonucleotides

HGF_F
TTTGGATCCACCATGTGGGTGACCAAACTCCTGCCA,
HGF_R
AATGTCGACCTATGACTGTGGTACCTTATATGTTAAAAT

and PCR amplification using the commercially available kit Phusion® High-Fidelity DNA Polymerase (New England Biolabs, USA) and constructed oligonucleotides.

The amplification product of the coding region of HGF gene and DNA vector VTvaf17 was cleaved by BamHI and SalI restriction endonucleases (New England Biolabs, USA).

This resulted in a 5349 bp DNA vector VTvaf17-HGF carrying the therapeutic gene, namely HGF gene, containing nucleotide sequence SEQ ID No. 6 allowing for antibiotic-free selection with the structure shown in FIG. 1.F.

Gene therapy DNA vector VTvaf17 was constructed as described in Example 1.

### Example 7

### Production of Gene Therapy DNA Vector VTvaf17-SDF1 Carrying the SDF1 Therapeutic Gene.

Gene therapy DNA vector VTvaf17-SDF1 was constructed by cloning the coding region of the SDF1 gene to the DNA vector VTvaf17 by BamHI-EcoRI restriction sites. The coding region of SDF1 gene (284 bp) was obtained by isolating total RNA from the human tissue biopsy sample followed by reverse transcription reaction using commercial kit Mint-2 (Evrogen, Russia) and constructed oligonucleotides

SDF_F
AGGATCCCACCATGAACGCCAAGGTCGTGGT,
SDF_R
TATGAATTCACATCTTGAACCTCTTGTTTAAAGC

and PCR amplification using the commercially available kit Phusion® High-Fidelity DNA Polymerase (New England Biolabs, USA) and constructed oligonucleotides.

The amplification product of the coding region of SDF1 gene and DNA vector VTvaf17 was cleaved by BamHI and EcoRI restriction endonucleases (New England Biolabs, USA).

This resulted in a 3425 bp DNA vector VTvaf17-SDF1 carrying the therapeutic gene, namely SDF1 gene, containing nucleotide sequence SEQ ID No. 7 allowing for antibiotic-free selection with the structure shown in FIG. 1.G.

Gene therapy DNA vector VTvaf17 was constructed as described in Example 1.

### Example 8

### Production of Gene Therapy DNA Vector VTvaf17-KLK4 Carrying the KLK4 Therapeutic Gene.

Gene therapy DNA vector VTvaf17-KLK4 was constructed by cloning the coding region of the KLK4 gene to the DNA vector VTvaf17 by SalI-EcoRI restriction sites. The coding region of KLK4 gene (769 bp) was obtained by isolating total RNA from the human tissue biopsy sample followed by reverse transcription reaction using commercial kit Mint-2 (Evrogen, Russia) and constructed oligonucleotides

KLK_F
TTTGTCGACCACCATGGCCACAGCAGGAAATCCC,
KLK_R
TTTTTGAATTCTTAACTGGCCTGGACGGTTTTCTC

and PCR amplification using the commercially available kit Phusion® High-Fidelity DNA Polymerase (New England Biolabs, USA) and constructed oligonucleotides.

The amplification product of the coding region of KLK4 gene and DNA vector VTvaf17 was cleaved by restriction endonucleases SalI and EcoRI (New England Biolabs, USA).

This resulted in a 3922 bp DNA vector VTvaf17-KLK4 carrying the therapeutic gene, namely KLK4 gene, containing nucleotide sequence SEQ ID No. 8 allowing for antibiotic-free selection with the structure shown in FIG. 1.H.

Gene therapy DNA vector VTvaf17 was constructed as described in Example 1.

### Example 9

### Production of Gene Therapy DNA Vector VTvaf17-PDGFC Carrying the PDGFC Therapeutic Gene.

Gene therapy DNA vector VTvaf17-PDGFC was constructed by cloning the coding region of the PDGFC gene to the DNA vector VTvaf17 by SalI-KpnI restriction sites. The coding region of PDGFC gene (1041 bp) was obtained by isolating total RNA from the human tissue biopsy sample followed by reverse transcription reaction using commercial kit Mint-2 (Evrogen, Russia) and constructed oligonucleotides

PDGFC_F
TTTGTCGACCACCATGAGCCTCTTCGGGCTTCTCC,
PDGFC_R
AATGGTACCTATCCTCCTGTGCTCCCTCTGCAC

and PCR amplification using the commercially available kit Phusion® High-Fidelity DNA Polymerase (New England Biolabs, USA) and constructed oligonucleotides.

The amplification product of the coding region of PDGFC gene and DNA vector VTvaf17 was cleaved by SalI and KpnI restriction endonucleases (New England Biolabs, USA).

This resulted in a 4200 bp DNA vector VTvaf17-PDGFC carrying the therapeutic gene, namely PDGFC gene, containing nucleotide sequence SEQ ID No. 9 allowing for antibiotic-free selection with the structure shown in FIG. 1.I.

Gene therapy DNA vector VTvaf17 was constructed as described in Example 1.

### Example 10

### Production of Gene Therapy DNA Vector VTvaf17-PROK1 Carrying the PROK1 Therapeutic Gene.

Gene therapy DNA vector VTvaf17-PROK1 was constructed by cloning the coding region of the PROK1 gene to the DNA vector VTvaf17 by SalI and KpnI restriction sites. The coding region of PROK1 gene (328 bp) was obtained by isolating total RNA from the human tissue biopsy sample followed by reverse transcription reaction using commercial kit Mint-2 (Evrogen, Russia) and constructed oligonucleotides

PROK1_F
TATGTCGACCACCATGAGAGGTGCCACGCGAG,
PROK1_R
TATGGAATTCGGTACGCTAAAAATTGATGTTCTTCAAGTCCA

and PCR amplification using the commercially available kit Phusion® High-Fidelity DNA Polymerase (New England Biolabs, USA) and constructed oligonucleotides.

The amplification product of the coding region of PROK1 gene and DNA vector VTvaf17 was cleaved by SalI and EcoRI restriction endonucleases (New England Biolabs, USA).

This resulted in a 3481 bp DNA vector VTvaf17-PROK1 carrying the therapeutic gene, namely PROK1 gene, containing nucleotide sequence SEQ ID No. 10 allowing for antibiotic-free selection with the structure shown in FIG. 1.K.

Gene therapy DNA vector VTvaf17 was constructed as described in Example 1.

### Example 11

### Production of Gene Therapy DNA Vector VTvaf17-PROK2 Carrying the PROK2 Therapeutic Gene.

Gene therapy DNA vector VTvaf17-PROK2 was constructed by cloning the coding region of the PROK2 gene to the DNA vector VTvaf17 by SalI-KpnI restriction sites. The coding region of PROK2 gene (394 bp) was obtained by isolating total RNA from the human tissue biopsy sample followed by reverse transcription reaction using commercial kit Mint-2 (Evrogen, Russia) and constructed oligonucleotides

PROK2_F
TTTGTCGACCACCATGAGGAGCCTGTGCTGCG,
PROK2_R
AATGGTACCTTACTTTTGGGCTAAACAAATAAATCGG

and PCR amplification using the commercially available kit Phusion® High-Fidelity DNA Polymerase (New England Biolabs, USA) and constructed oligonucleotides.

The amplification product of the coding region of PROK2 gene and DNA vector VTvaf17 was cleaved by SalI and KpnI restriction endonucleases (New England Biolabs, USA).

This resulted in a 3553 bp DNA vector VTvaf17-PROK2 carrying the therapeutic gene, namely PPROK2 gene, containing nucleotide sequence SEQ ID No. 11 allowing for antibiotic-free selection with the structure shown in FIG. 1.L.

Gene therapy DNA vector VTvaf17 was constructed as described in Example 1.

### Example 12

Proof of the efficiency of gene therapy DNA vector VTvaf17-ANG carrying the ANG therapeutic gene. This example also demonstrates practicability of use of gene therapy DNA vector carrying ANG therapeutic gene.

To confirm the efficiency of gene therapy DNA vector VTvaf17-ANG, changes in mRNA accumulation of the ANG therapeutic gene in HDFa human primary dermal fibroblast cells ATCCPCS-201-012) 48 hours after their transfection with gene therapy DNA vector VTvaf17-ANG were assessed.

HDFa human primary dermal fibroblast cells were grown in Fibroblast Basal Medium (ATCC PCS-201-030) with the addition of components included in the Fibroblast Growth Kit-Serum-Free (ATCC PCS-201-040) at 37° C. in the presence of 5% CO2. To achieve 90% confluence, 24 hours before the transfection procedure, the cells were seeded into a 24-well plate in the quantity of 5×10⁴ cells per well.

Lipofectamine 3000 (ThermoFisher Scientific, USA) was used as a transfection reagent. The transfection with gene therapy DNA vector VTvaf17-ANG was performed as follows. In test tube 1, 1 μl of DNA vector VTvaf17-ANG solution (concentration 500 ng/μl) and 1 μl of reagent P3000 was added to 25 μl of medium Opti-MEM (Gibco). The preparation was mixed by gentle shaking. In test tube 2, 1 μl of Lipofectamine 3000 solution was added to 25 μl of medium Opti-MEM (Gibco). The preparation was mixed by gentle shaking. The contents from test tube 1 were added to the contents of test tube 2, and the mixture was incubated at room temperature for 5 minutes. The resulting solution was added dropwise to the cells in the volume of 40 μl.

HDFa Human dermal fibroblasts transfected with the gene therapy DNA vector VTvaf17 devoid of the inserted therapeutic gene (cDNA of ANG gene before and after transfection with gene therapy DNA vector VTvaf17 devoid of the inserted therapeutic gene is not shown in the figures) were used as a reference. Reference vector VTvaf17 for transfection was prepared as described above.

Extraction of total RNA from the transfected cells was performed as follows. 1 ml of Trizol Reagent (ThermoFisher Scientific) was added to the well with cells, homogenised and heated for 5 minutes at 65° C. The sample was centrifuged at 14,000 g for 10 minutes and heated again for 10 minutes at 65° C. Then 200 μl of chloroform was added, and the mixture was gently stirred and centrifuged at 14,000 g for 10 minutes. Then the water phase was isolated and mixed with 1/10 of the volume of 3M sodium acetate, pH 5.2, and an equal volume of isopropyl alcohol. The sample was incubated at −20° C. for 10 minutes and then centrifuged at 14,000 g for 10 minutes. The precipitated RNA were rinsed in 1 ml of 70% ethyl alcohol, air-dried, and dissolved in 10 μl of RNase-free water. To measure the mRNA expression level of ANG gene after transfection, real-time PCR method (SYBR Green Real Time PCR) was used. For the amplification of cDNA specific for ANG human gene, the following oligonucleotides were used:

ANG_SF
TGGGCGTTTTGTTGTTGGTC,
ANG_FR
TGTCTTTGCAGGGTGAGGTC

the length of amplification product is 183 bp. Beta-2 microglobulin (B2M) was used as a reference gene.

PCR amplification was performed using SYBR GreenQuantitect RT-PCR Kit (Qiagen, USA) in real-time in 20 μl of the amplification mixture containing: 25 μl of QuantiTect SYBR Green RT-PCR Master Mix, 2.5 mM of magnesium chloride, 0.5 μM of each primer, and 5 μl of total RNA. For the reaction, CFX96 amplifier (Bio-Rad, USA) was used under the following conditions: 1 cycle of reverse transcription at 42° C. for 30 minutes, denaturation at 98° C. for 15 minutes, followed by 40 cycles comprising denaturation at 94° C. for 15 s, annealing of primers at 60° C. for 30 s and elongation at 72° C. for 120 s. Positive control included amplicons from PCR on matrices represented by plasmids in known concentrations containing cDNA sequences of ANG and B2M genes. Negative control included deionised water. Real-time quantification of the PCR products, i.e. ANG and B2M gene cDNAs obtained by amplification, was conducted using the Bio-Rad CFX Manager 2.1 software.

To confirm increased expression of the ANG gene in HDFa human primary dermal fibroblast cells after transfection of these cells with gene therapy DNA vector VTvaf17-ANG, FIG. 2 shows diagrams of accumulation of PCR products that indicate that due to the transfection of HDFa human primary dermal fibroblast cells with gene therapy DNA vector VTvaf17-ANG, the level of specific mRNA of the human ANG gene has grown massively. This demonstrates the efficiency of gene therapy DNA vector VTvaf17-ANG. The presented results also confirm the practicability of use of gene therapy DNA vector VTvaf17-ANG in order to increase the expression level of ANG gene in eukaryotic cells.

### Example 13

Proof of the efficiency of gene therapy DNA vector ANGPT1 carrying the ANGPT1 therapeutic gene. This example also demonstrates practicability of use of gene therapy DNA vector carrying the ANGPT1 therapeutic gene.

To confirm the efficiency of gene therapy DNA vector VTvaf17-ANGPT1, changes in mRNA accumulation of the ANGPT1 therapeutic gene in HT 297.T human primary dermal fibroblast cells (ATCC® CRL-7782™) 48 hours after their transfection with gene therapy DNA vector VTvaf17-ANGPT1 were assessed.

HT 297.T human primary dermal fibroblast cells were grown in Dulbecco's Modified Eagle's Medium according to the manufacturer's method (https://www.1gestandards-atcc.org/products/all/CRL-7782.aspx#culturemethod) at 37° C. in the presence of 5% CO2. To achieve 90% confluence, 24 hours before the transfection procedure, the cells were seeded into a 24-well plate in the quantity of 5×10⁴ cells per well.

HT 297.T human primary dermal fibroblast cells were transfected as described in Example 12.

The transfection was performed with gene therapy DNA vector VTvaf17-ANGPT1.

HT 297.T human dermal fibroblasts transfected with the gene therapy DNA vector ANGPT1 devoid of the inserted therapeutic gene (cDNA of ANGPT1 gene before and after transfection with gene therapy DNA vector VTvaf17 devoid of the inserted therapeutic gene is not shown in the figures) were used as a reference. Reference vector VTvaf17 for transfection was prepared as described in Example 12.

Total RNA from transfected cells was isolated as described in Example 12. To measure the mRNA expression level of ANGPT1 gene after transfection, real-time PCR method (SYBR Green Real Time PCR) was used. For the amplification of cDNA specific for the human ANGPT1 gene, the following oligonucleotides were used

ANGPT1_SF
TGCAGAGAGATGCTCCACAC,
ANGPT1_FR
ATGGTAGCCGTGTGGTTCTG

The length of amplification product is 181 bp. Beta-2 microglobulin (B2M) was used as a reference gene.

PCR amplification was performed as described in Example 12 under the following conditions: 1 cycle of reverse transcription at 42° C. for 30 minutes, denaturation at 98° C. for 15 minutes, followed by 40 cycles comprising denaturation at 94° C. for 15 s, annealing of primers at 60° C. for 30 s and elongation at 72° C. for 120 s. Positive control included amplicons from PCR on matrices represented by plasmids in known concentrations containing cDNA sequences of ANGPT1 and B2M genes. Negative control included deionised water. Real-time quantification of the PCR products, i.e. ANGPT1 and B2M gene cDNAs obtained by amplification, was conducted using the Bio-Rad CFX Manager 2.1 software.

To confirm increased expression of the ANGPT1 gene in HT 297.T human primary dermal fibroblast cells after transfection of these cells with gene therapy DNA vector VTvaf17-ANGPT1, FIG. 3 shows diagrams of accumulation of PCR products that indicate that due to the transfection of HT 297.T human primary dermal fibroblast cells with gene therapy DNA vector VTvaf17-ANGPT1, the level of specific mRNA of the human ANGPT1 gene has grown massively. This demonstrates the efficiency of gene therapy DNA vector VTvaf17-ANGPT1. The presented results also confirm the practicability of use of gene therapy DNA vector VTvaf17-ANGPT1 in order to increase the expression level of ANGPT1 gene in eukaryotic cells.

### Example 14

Proof of the efficiency of gene therapy DNA vector VTvaf17-VEGFA carrying the VEGFA therapeutic gene. This example also demonstrates practicability of use of gene therapy DNA vector carrying the VEGFA therapeutic gene.

To confirm the efficiency of gene therapy DNA vector VTvaf17-VEGFA, changes in mRNA accumulation of the VEGFA therapeutic gene in Hs27 human primary foreskin fibroblast cells (ATCC® CRL-1634™) 48 hours after their transfection with gene therapy DNA vector VTvaf17-VEGFA were assessed.

Hs27 human primary foreskin fibroblast cells were grown in Dulbecco's Modified Eagle's Medium according to the manufacturer's method (https://www.1gcstandards-atcc.org/products/all/CRL-1634.aspx#culturemethod) at 37° C. in the presence of atmosphere. To achieve 90% confluence, 24 hours before the transfection procedure, the cells were seeded into a 24-well plate in the quantity of 5×10⁴ cells per well.

Hs27 human primary foreskin fibroblast cells were transfected as described in Example 12.

The transfection was performed with gene therapy DNA vector VTvaf17-VEGFA.

Hs27 human primary foreskin fibroblast cells transfected with the gene therapy DNA vector VTvaf17 devoid of the inserted therapeutic gene (cDNA of VEGFA gene before and after transfection with gene therapy DNA vector VTvaf17 devoid of the inserted therapeutic gene is not shown in the figures) were used as a reference. Reference vector VTvaf17 for transfection was prepared as described in Example 12.

Total RNA from transfected cells was isolated as described in Example 12. To measure the mRNA expression level of VEGFA gene after transfection, real-time PCR method (SYBR Green Real Time PCR) was used. For the amplification of cDNA specific for the human VEGFA gene, the following oligonucleotides were used

VEGFA_SF
TCTGCTGTCTTGGGTGCATT
VEGFA_FR
CCAGGGTCTCGATTGGATGG

The length of amplification product is 167 bp. Beta-2 microglobulin (B2M) was used as a reference gene.

PCR amplification was performed as described in Example 12 under the following conditions: 1 cycle of reverse transcription at 42° C. for 30 minutes, denaturation at 98° C. for 15 minutes, followed by 40 cycles comprising denaturation at 94° C. for 15 s, annealing of primers at 60° C. for 30 s and elongation at 72° C. for 120 s. Positive control included amplicons from PCR on matrices represented by plasmids in known concentrations containing cDNA sequences of VEGFA and B2M genes. Negative control included deionised water. Real-time quantification of the PCR products, i.e. VEGFA and B2M genes cDNAs obtained by amplification, was conducted using the Bio-Rad CFX Manager 2.1 software.

To confirm increased expression of the VEGFA gene in Hs27 foreskin fibroblast cell culture after transfection of these cells with gene therapy DNA vector VTvaf17-VEGFA, FIG. 4 shows diagrams of accumulation of PCR products that indicate that due to the transfection of Hs27 human primary foreskin fibroblast cell with gene therapy DNA vector VTvaf17-VEGFA, the level of specific mRNA of the human VEGFA gene has grown massively. This demonstrates the efficiency of gene therapy DNA vector VTvaf17-VEGFA. The presented results also confirm the practicability of use of gene therapy DNA vector VTvaf17-VEGFA in order to increase the expression level of VEGFA gene in eukaryotic cells.

### Example 15

Proof of the efficiency of gene therapy DNA vector VTvaf17-FGF1 carrying FGF1 therapeutic gene. This example also demonstrates practicability of use of gene therapy DNA vector carrying the FGF1 therapeutic gene.

To confirm the efficiency of gene therapy DNA vector VTvaf17-FGF1, changes in mRNA accumulation of the FGF1 therapeutic gene in HSkM human primary skeletal muscle myoblast cells (A12555, Thermo Fisher Scientific) 48 hours after their transfection with gene therapy DNA vector VTvaf17-FGF1 were assessed.

HSkM human primary skeletal muscle myoblast cells were grown in Gibco® HSkM Differentiation Medium (DM) according to the manufacturer's method (https://www.thermofisher.com/order/catalog/product/A12555) at 37° C. in the presence of 5% CO2. To achieve 90% confluence, 24 hours before the transfection procedure, the cells were seeded into a 24-well plate in the quantity of 5×10⁴ cells per well.

HSkM human primary skeletal muscle myoblast cells were transfected as described in Example 12.

The transfection was performed with gene therapy DNA vector VTvaf17-FGF1.

HSkM human skeletal muscle myoblast cells transfected with the gene therapy DNA vector VTvaf17 devoid of the inserted therapeutic gene (cDNA of FGF1 gene before and after transfection with gene therapy DNA vector VTvaf17 devoid of the inserted therapeutic gene is not shown in the figures) were used as a reference. Reference vector VTvaf17 for transfection was prepared as described in Example 12.

Total RNA from transfected cells was isolated as described in Example 12. To measure the mRNA expression level of FGF1 gene after transfection, real-time PCR method (SYBR Green Real Time PCR) was used. For the amplification of cDNA specific for FGF1 human gene, the following oligonucleotides were used

FGF_SF
CAGTGGATGGGACAAGGGAC,
FGF_FR
GGTTCTCCTCCAGCCTTTCC

The length of amplification product is 189 bp. Beta-2 microglobulin (B2M) was used as a reference gene.

PCR amplification was performed as described in Example 12 under the following conditions: 1 cycle of reverse transcription at 42° C. for 30 minutes, denaturation at 98° C. for 15 minutes followed by 40 cycles comprising denaturation at 94° C. for 15 s, annealing of primers at 60° C. for 30 s and elongation at 72° C. for 120 s. Positive control included amplicons from PCR on matrices represented by plasmids in known concentrations containing cDNA sequences of FGF1 and B2M genes. Negative control included deionised water. Real-time quantification of the PCR products, i.e. FGF1 and B2M gene cDNAs obtained by amplification, was conducted using the Bio-Rad CFX Manager 2.1 software.

To confirm increased expression of the FGF1 gene in HSkM human skeletal muscle myoblast cell culture after transfection of these cells with gene therapy DNA vector VTvaf17-FGF1, FIG. 5 shows diagrams of accumulation of PCR products that indicate that due to the transfection of HSkM human primary skeletal muscle myoblast cells with gene therapy DNA vector VTvaf17-FGF1, the level of specific mRNA of the human FGF1 gene has grown massively. This demonstrates the efficiency of gene therapy DNA vector VTvaf17-FGF1. The presented results also confirm the practicability of use of gene therapy DNA vector VTvaf17-FGF1 in order to increase the expression level of FGF1 gene in eukaryotic cells.

### Example 16

Proof of the efficiency of gene therapy DNA vector VTvaf17-HIF1α carrying HIF1α therapeutic gene. This example also demonstrates practicability of use of gene therapy DNA vector carrying the HIF1α therapeutic gene.

To confirm the efficiency of gene therapy DNA vector VTvaf17-HIF1α, changes in mRNA accumulation of the HIF1α therapeutic gene in HBdSMc human primary bladder smooth muscle cells (ATCC® PCS-420-012™) 48 hours after their transfection with gene therapy DNA vector VTvaf17-HIF1α were assessed.

HBdSMc human primary bladder smooth muscle cells were grown in Vascular Cell Basal Medium (ATCC PCS-100-030) with the addition of components included in the Growth Kit (ATCC PCS-100-042) at 37° C. in the presence of 5% CO2. To achieve 90% confluence, 24 hours before the transfection procedure, the cells were seeded into a 24-well plate in the quantity of 5×10⁴ cells per well.

HBdSMc human primary bladder smooth muscle cells were transfected as described in Example 12.

The transfection was performed with gene therapy DNA vector VTvaf17-HIF1α.

HBdSMc human primary bladder smooth muscle cells transfected with the gene therapy DNA vector VTvaf17 devoid of the inserted therapeutic gene (cDNA of HIF1α gene before and after transfection with gene therapy DNA vector VTvaf17 devoid of the inserted therapeutic gene is not shown in the figures) were used as a reference. Reference vector VTvaf17 for transfection was prepared as described in Example 12.

Total RNA from transfected cells was isolated as described in Example 12. To measure the mRNA expression level of HIF1α gene after transfection, real-time PCR method (SYBR Green Real Time PCR) was used. For the amplification of cDNA specific for the human HIF1α gene, the following oligonucleotides were used

HIF_SF
TTTTGGCAGCAACGACACAG,
HIF_FR
GTGCAGGGTCAGCACTACTT

The length of amplification product is 173 bp. Beta-2 microglobulin (B2M) was used as a reference gene.

PCR amplification was performed as described in Example 12 under the following conditions: 1 cycle of reverse transcription at 42° C. for 30 minutes, denaturation at 98° C. for 15 minutes followed by 40 cycles comprising denaturation at 94° C. for 15 s, annealing of primers at 60° C. for 30 s and elongation at 72° C. for 120 s. Positive control included amplicons from PCR on matrices represented by plasmids in known concentrations containing cDNA sequences of HIF1α and B2M genes. Negative control included deionised water. Real-time quantification of the PCR products, i.e. HIF1α and B2M gene cDNAs obtained by amplification, was conducted using the Bio-Rad CFX Manager 2.1 software.

To confirm increased expression of the HIF1α gene in HBdSMc human primary bladder smooth muscle cells after transfection of these cells with gene therapy DNA vector VTvaf17-HIF1α, FIG. 6 shows diagrams of accumulation of PCR products that indicate that due to the transfection of HBdSMc human primary bladder smooth muscle cells with gene therapy DNA vector VTvaf17-HIF1α, the level of specific mRNA of the human HIF1α gene has grown massively. This demonstrates the efficiency of gene therapy DNA vector VTvaf17-HIF1α. The presented results also confirm the practicability of use of gene therapy DNA vector VTvaf17-HIF1α in order to increase the expression level of HIF1α gene in eukaryotic cells.

### Example 17

Proof of the efficiency of gene therapy DNA vector VTvaf17-HGF carrying the HGF therapeutic gene. This example also demonstrates practicability of use of gene therapy DNA vector carrying HGF therapeutic gene.

To confirm the efficiency of gene therapy DNA vector VTvaf17-HGF, changes in mRNA accumulation of the HGF therapeutic gene in T/GHA-VSMC primary aortic smooth muscle cells (ATCC® CRL-1999™) 48 hours after their transfection with gene therapy DNA vector VTvaf17-HGF were assessed.

T/GHA-VSMC primary aortic smooth muscle cells were grown in F-12K Medium according to the manufacturer's method (https://www.1gcstandards-atcc.org/products/all/CRL-1999.aspx#culturemethod) at 37° C. in the presence of atmosphere. To achieve 90% confluence, 24 hours before the transfection procedure, the cells were seeded into a 24-well plate in the quantity of 5×10⁴ cells per well.

T/GHA-VSMC primary aortic smooth muscle cells were transfected as described in Example 12.

The transfection was performed with gene therapy DNA vector VTvaf17-HGF.

T/GHA-VSMC primary aortic smooth muscle cells transfected with the gene therapy DNA vector VTvaf17 devoid of the inserted therapeutic gene (cDNA of HGF gene before and after transfection with gene therapy DNA vector VTvaf17 devoid of the inserted therapeutic gene is not shown in the figures) were used as a reference. Reference vector VTvaf17 for transfection was prepared as described in Example 12.

Total RNA from transfected cells was isolated as described in Example 12. To measure the mRNA expression level of HGF gene after transfection, real-time PCR method (SYBR Green Real Time PCR) was used. For the amplification of cDNA specific for the human HGF gene, the following oligonucleotides were used

HGF_SF
ACCCTGGTGTTTCACAAGCA,
HGF_FR
GCAAGAATTTGTGCCGGTGT

The length of amplification product is 182 bp. Beta-2 microglobulin (B2M) was used as a reference gene.

PCR amplification was performed as described in Example 12 under the following conditions: 1 cycle of reverse transcription at 42° C. for 30 minutes, denaturation at 98° C. for 15 minutes, followed by 40 cycles comprising denaturation at 94° C. for 15 s, annealing of primers at 60° C. for 30 s and elongation at 72° C. for 120 s. Positive control included amplicons from PCR on matrices represented by plasmids in known concentrations containing cDNA sequences of HGF and B2M genes. Negative control included deionised water. Real-time quantification of the PCR products, i.e. HGF and B2M gene cDNAs obtained by amplification, was conducted using the Bio-Rad CFX Manager 2.1 software.

To confirm increased expression of the HGF gene in T/GHA-VSMC primary aortic smooth muscle cells after transfection of these cells with gene therapy DNA vector VTvaf17-HGF, FIG. 7 shows diagrams of accumulation of PCR products that indicate that due to the transfection of T/GHA-VSMC primary aortic smooth muscle cells with gene therapy DNA vector VTvaf17-HGF, the level of specific mRNA of the human HGF gene has grown massively. This demonstrates the efficiency of gene therapy DNA vector VTvaf17-HGF. The presented results also confirm the practicability of use of gene therapy DNA vector VTvaf17-HGF in order to increase the expression level of HGF gene in eukaryotic cells.

### Example 18

Proof of the efficiency of gene therapy DNA vector VTvaf17-SDF1 carrying SDF1 therapeutic gene. This example also demonstrates practicability of use of gene therapy DNA vector carrying the SDF1 therapeutic gene.

To confirm the efficiency of gene therapy DNA vector VTvaf17-SDF1, changes in mRNA accumulation of the SDF1 therapeutic gene in HEKa primary epidermal keratinocytes (ATCC® PCS-200-011™) 48 hours after their transfection with gene therapy DNA vector VTvaf17-SDF1 were assessed.

HEKa primary epidermal keratinocytes were grown in Dermal Cell Basal Medium (ATCC® PCS200030) with the addition of Keratinocyte Growth Kit (ATCC® PCS200040) at 37° C. in the presence of 5% CO2. To achieve 90% confluence, 24 hours before the transfection procedure, the cells were seeded into a 24-well plate in the quantity of 5×104 cells per well.

HEKa primary epidermal keratinocytes were transfected as described in Example 12.

The transfection was performed with gene therapy DNA vector VTvaf17-SDF1.

HEKa epidermal keratinocytes transfected with the gene therapy DNA vector VTvaf17 devoid of the inserted therapeutic gene (cDNA of SDF1 gene before and after transfection with gene therapy DNA vector VTvaf17 devoid of the inserted therapeutic gene is not shown in the figures) were used as a reference. Reference vector VTvaf17 for transfection was prepared as described in Example 12.

Total RNA from transfected cells was isolated as described in Example 12. To measure the mRNA expression level of SDF1 gene after transfection, real-time PCR method (SYBR Green Real Time PCR) was used. For the amplification of cDNA specific for the human SDF1 gene, the following oligonucleotides were used

SDF_SF
TGAGCTACAGATGCCCATGC,
SDF_FR
TAGCTTCGGGTCAATGCACA

The length of amplification product is 152 bp. Beta-2 microglobulin (B2M) was used as a reference gene.

PCR amplification was performed as described in Example 12 under the following conditions: 1 cycle of reverse transcription at 42° C. for 30 minutes, denaturation at 98° C. for 15 minutes followed by 40 cycles comprising denaturation at 94° C. for 15 s, annealing of primers at 60° C. for 30 s and elongation at 72° C. for 120 s. Positive control included amplicons from PCR on matrices represented by plasmids in known concentrations containing cDNA sequences of SDF1 and B2M genes. Negative control included deionised water. Real-time quantification of the PCR products, i.e. SDF1 and B2M gene cDNAs obtained by amplification, was conducted using the Bio-Rad CFX Manager 2.1 software.

To confirm increased expression of the SDF1 gene in HEKa epidermal keratinocyte culture after transfection of these cells with gene therapy DNA vector VTvaf17-SDF1, FIG. 8 shows diagrams of accumulation of PCR products that indicate that due to the transfection of HEKa primary epidermal keratinocyte culture with gene therapy DNA vector VTvaf17-SDF1, the level of specific mRNA of the human SDF1 gene has grown massively. This demonstrates the efficiency of gene therapy DNA vector VTvaf17-SDF1. The presented results also confirm the practicability of use of gene therapy DNA vector VTvaf17-SDF1 in order to increase the expression level of SDF1 gene in eukaryotic cells.

### Example 19

Proof of the efficiency of gene therapy DNA vector VTvaf17-KLK4 carrying the KLK4 therapeutic gene. This example also demonstrates practicability of use of gene therapy DNA vector carrying the KLK4 therapeutic gene.

To confirm the efficiency of gene therapy DNA vector VTvaf17-KLK4, changes in mRNA accumulation of the KLK4 therapeutic gene in HUVEC primary umbilical vein endothelial cells (ATCC® PCS-100-010™) 48 hours after their transfection with gene therapy DNA vector VTvaf17-KLK4 were assessed.

HUVEC primary umbilical vein endothelial cells were grown in Vascular Cell Basal Medium (ATCC PCS-100-030) according to the manufacturer's method (https://www.1gestandards-atcc.org/products/all/PCS-100-010.aspx#cultureconditions) at 37° C. in the presence of 5% CO2. To achieve 90% confluence, 24 hours before the transfection procedure, the cells were seeded into a 24-well plate in the quantity of 5×104 cells per well.

HUVEC primary umbilical vein endothelial cells were transfected as described in Example 12.

The transfection was performed with gene therapy DNA vector VTvaf17-KLK4.

HUVEC primary umbilical vein endothelial cells transfected with the gene therapy DNA vector VTvaf17 devoid of the inserted therapeutic gene (cDNA of KLK4 gene before and after transfection with gene therapy DNA vector VTvaf17 devoid of the inserted therapeutic gene is not shown in the figures) were used as a reference. Reference vector VTvaf17 for transfection was prepared as described in Example 12.

Total RNA from transfected cells was isolated as described in Example 12. To measure the mRNA expression level of KLK4 gene after transfection, real-time PCR method (SYBR Green Real Time PCR) was used. For the amplification of cDNA specific for the human KLK4 gene, the following oligonucleotides were used

KLK_SF
CGGAGCATCAGCATTGCTTC,
KLK_FR
GAACATGCTGGGGTGGTACA

The length of amplification product is 177 bp. Beta-2 microglobulin (B2M) was used as a reference gene.

PCR amplification was performed as described in Example 12 under the following conditions: 1 cycle of reverse transcription at 42° C. for 30 minutes, denaturation at 98° C. for 15 minutes followed by 40 cycles comprising denaturation at 94° C. for 15 s, annealing of primers at 60° C. for 30 s and elongation at 72° C. for 120 s. Positive control included amplicons from PCR on matrices represented by plasmids in known concentrations containing cDNA sequences of KLK4 and B2M genes. Negative control included deionised water. Real-time quantification of the PCR products, i.e. KLK4 and B2M gene cDNAs obtained by amplification, was conducted using the Bio-Rad CFX Manager 2.1 software.

To confirm increased expression of the KLK4 gene in HUVEC primary umbilical vein endothelial cells after transfection of these cells with gene therapy DNA vector VTvaf17-KLK4, FIG. 9 shows diagrams of accumulation of PCR products that indicate that due to the transfection of HUVEC primary umbilical vein endothelial cells with gene therapy DNA vector VTvaf17-KLK4, the level of specific mRNA of the human KLK4 gene has grown massively. This demonstrates the efficiency of gene therapy DNA vector VTvaf17-KLK4. The presented results also confirm the practicability of use of gene therapy DNA vector VTvaf17-KLK4 in order to increase the expression level of KLK4 gene in eukaryotic cells.

### Example 20

Proof of the efficiency of gene therapy DNA vector VTvaf17-PDGFC carrying the PDGFC therapeutic gene. This example also demonstrates practicability of use of gene therapy DNA vector carrying the PDGFC therapeutic gene.

To confirm the efficiency of gene therapy DNA vector VTvaf17-PDGFC, changes in mRNA accumulation of the PDGFC therapeutic gene in HEMa primary epidermal melanocyte cells (ATCC® PCS-200-013™) 48 hours after their transfection with gene therapy DNA vector VTvaf17-PDGFC were assessed.

HEMa primary epidermal melanocyte cells were grown in Dermal Cell Basal Medium (ATCC® PCS200030) according to the manufacturer's method (https://www.1gcstandards-atcc.org/products/all/PCS-200-013.aspx#cultureconditions) at 37° C. in the presence of 5% CO2. To achieve 90% confluence, 24 hours before the transfection procedure, the cells were seeded into a 24-well plate in the quantity of 5×104 cells per well.

HEMa primary epidermal melanocyte cells were transfected as described in Example 12.

The transfection was performed with gene therapy DNA vector VTvaf17-PDGFC.

HEMa primary epidermal melanocyte cells transfected with the gene therapy DNA vector VTvaf17 devoid of the inserted therapeutic gene (cDNA of PDGFC gene before and after transfection with gene therapy DNA vector VTvaf17 devoid of the inserted therapeutic gene is not shown in the figures) were used as a reference. Reference vector VTvaf17 for transfection was prepared as described in Example 12.

Total RNA from transfected cells was isolated as described in Example 12. To measure the mRNA expression level of PDGFC gene after transfection, real-time PCR method (SYBR Green Real Time PCR) was used. For the amplification of cDNA specific for the human PDGFC gene, the following oligonucleotides were used

PDGFC_SF
ATATTAGGGCGCTGGTGTGG,
PDGFC_FR
AGCACTGAAGGACTCACAGC

The length of amplification product is 173 bp. Beta-2 microglobulin (B2M) was used as a reference gene.

PCR amplification was performed as described in Example 12 under the following conditions: 1 cycle of reverse transcription at 42° C. for 30 minutes, denaturation at 98° C. for 15 minutes followed by 40 cycles comprising denaturation at 94° C. for 15 s, annealing of primers at 60° C. for 30 s and elongation at 72° C. for 120 s. Positive control included amplicons from PCR on matrices represented by plasmids in known concentrations containing cDNA sequences of PDGFC and B2M genes. Negative control included deionised water. Real-time quantification of the PCR products, i.e. PDGFC and B2M gene cDNAs obtained by amplification, was conducted using the Bio-Rad CFX Manager 2.1 software.

To confirm increased expression of the PDGFC gene in HEMa epidermal melanocyte cell culture after transfection of these cells with gene therapy DNA vector VTvaf17-PDGFC, FIG. 10 shows diagrams of accumulation of PCR products that indicate that due to the transfection of HEMa primary epidermal melanocyte cell culture with gene therapy DNA vector VTvaf17-PDGFC, the level of specific mRNA of the human PDGFC gene has grown massively. This demonstrates the efficiency of gene therapy DNA vector VTvaf17-PDGFC. The presented results also confirm the practicability of use of gene therapy DNA vector VTvaf17-PDGFC in order to increase the expression level of PDGFC gene in eukaryotic cells.

### Example 21

Proof of the efficiency of gene therapy DNA vector VTvaf17-PROK1 carrying the PROK1 therapeutic gene. This example also demonstrates practicability of use of gene therapy DNA vector carrying the PROK1 therapeutic gene.

To confirm the efficiency of gene therapy DNA vector VTvaf17-PROK1, changes in mRNA accumulation of the PROK1 therapeutic gene in HSkM human primary skeletal muscle myoblast cells (A12555, Thermo Fisher Scientific) 48 hours after their transfection with gene therapy DNA vector VTvaf17-PROK1 were assessed.

HSkM human primary skeletal muscle myoblast cells were grown in Gibco® HSkM Differentiation Medium (DM) according to the manufacturer's method (https://www.thermofisher.com/order/catalog/product/A1255.5) at 37° C. in the presence of 5% CO2. To achieve 90% confluence, 24 hours before the transfection procedure, the cells were seeded into a 24-well plate in the quantity of 5×10⁴ cells per well.

HSkM human primary skeletal muscle myoblast cells were transfected as described in Example 12.

The transfection was performed with gene therapy DNA vector VTvaf17-PROK1.

HSkM primary skeletal muscle myoblast cells transfected with the gene therapy DNA vector VTvaf17 devoid of the inserted therapeutic gene (cDNA of PROK1 gene before and after transfection with gene therapy DNA vector VTvaf17 devoid of the inserted therapeutic gene is not shown in the figures) were used as a reference. Reference vector VTvaf17 for transfection was prepared as described in Example 12.

Total RNA from transfected cells was isolated as described in Example 12. To measure the mRNA expression level of PROK1 gene after transfection, real-time PCR method (SYBR Green Real Time PCR) was used. For the amplification of cDNA specific for the human PROK1 gene, the following oligonucleotides were used

PROK1_SF
ATCAGCCTGTGGCTTCGAG,
PROK1_SR
TCAAGTCCATGGAGCAGCG

The length of amplification product is 184 bp. Beta-2 microglobulin (B2M) was used as a reference gene.

PCR amplification was performed as described in Example 12 under the following conditions: 1 cycle of reverse transcription at 42° C. for 30 minutes, denaturation at 98° C. for 15 minutes followed by 40 cycles comprising denaturation at 94° C. for 15 s, annealing of primers at 60° C. for 30 s and elongation at 72° C. for 120 s. Positive control included amplicons from PCR on matrices represented by plasmids in known concentrations containing cDNA sequences of PROK1 and B2M genes. Negative control included deionised water. Real-time quantification of the PCR products, i.e. PROK1 and B2M gene cDNAs obtained by amplification, was conducted using the Bio-Rad CFX Manager 2.1 software.

To confirm increased expression of the PROK1 gene in HSkM human skeletal muscle myoblast cell culture after transfection of these cells with gene therapy DNA vector VTvaf17-PROK1, FIG. 11 shows diagrams of accumulation of PCR products that indicate that due to the transfection of HSkM human primary skeletal muscle myoblast cells with gene therapy DNA vector VTvaf17-PROK1, the level of specific mRNA of the human PROK1 gene has grown massively. This demonstrates the efficiency of gene therapy DNA vector VTvaf17-PROK1. The presented results also confirm the practicability of use of gene therapy DNA vector VTvaf17-PROK1 in order to increase the expression level of PROK1 gene in eukaryotic cells.

### Example 22

Proof of the efficiency of gene therapy DNA vector VTvaf17-PROK2 carrying the PROK2 therapeutic gene. This example also demonstrates practicability of use of gene therapy DNA vector carrying the PROK2 therapeutic gene.

To confirm the efficiency of gene therapy DNA vector VTvaf17-PROK2, changes in mRNA accumulation of the PROK2 therapeutic gene in HMEC-1 primary dermal microvascular endothelial cells (ATCC® CRL-3243™) 48 hours after their transfection with gene therapy DNA vector VTvaf17-PROK2 were assessed.

HMEC-1 primary dermal microvascular endothelial cells were grown in MCDB131 (without L-Glutamine) medium according to the manufacturer's method (https://www.1gcstandards-atcc.org/products/all/CRL-3243.aspx#culturemethod) at 37° C. To achieve 90% confluence, 24 hours before the transfection procedure, the cells were seeded into a 24-well plate in the quantity of 5×10⁴ cells per well.

HMEC-1 primary dermal microvascular endothelial cells were transfected as described in Example 12.

The transfection was performed with gene therapy DNA vector VTvaf17-PROK2.

HMEC-1 dermal microvascular endothelial cells transfected with the gene therapy DNA vector VTvaf17 devoid of the inserted therapeutic gene (cDNA of PROK2 gene before and after transfection with gene therapy DNA vector VTvaf17 devoid of the inserted therapeutic gene is not shown in the figures to simplify visualisation) were used as a reference. Reference vector VTvaf17 for transfection was prepared as described in Example 12.

Total RNA from transfected cells was isolated as described in Example 12. To measure the mRNA expression level of PROK2 gene after transfection, real-time PCR method (SYBR Green Real Time PCR) was used. For the amplification of cDNA specific for the human PROK2 gene, the following oligonucleotides were used

PROK2_SF
ATGGGCAAACTGGGAGACAG,
PROK2_SF
ATGGGCAAACTGGGAGACAG

The length of amplification product is 174 bp. Beta-2 microglobulin (B2M) was used as a reference gene.

PCR amplification was performed as described in Example 12 under the following conditions: 1 cycle of reverse transcription at 42° C. for 30 minutes, denaturation at 98° C. for 15 minutes followed by 40 cycles comprising denaturation at 94° C. for 15 s, annealing of primers at 60° C. for 30 s and elongation at 72° C. for 120 s. Positive control included amplicons from PCR on matrices represented by plasmids in known concentrations containing cDNA sequences of PROK2 and B2M genes. Negative control included deionised water. Real-time quantification of the PCR products, i.e. PROK2 and B2M gene cDNAs obtained by amplification, was conducted using the Bio-Rad CFX Manager 2.1 software.

To confirm increased expression of the PROK2 gene in HMEC-1 dermal microvascular endothelial cell culture after transfection of these cells with gene therapy DNA vector VTvaf17-PROK2, FIG. 12 shows diagrams of accumulation of PCR products that indicate that due to the transfection of HMEC-1 primary dermal microvascular endothelial cells with gene therapy DNA vector VTvaf17-PROK2, the level of specific mRNA of the human PROK2 gene has grown massively. This demonstrates the efficiency of gene therapy DNA vector VTvaf17-PROK2. The presented results also confirm the practicability of use of gene therapy DNA vector VTvaf17-PROK2 in order to increase the expression level of PROK2 gene in eukaryotic cells.

### Example 23

Proof of the efficiency of gene therapy DNA vector VTvaf17-ANG carrying the therapeutic gene, namely the ANG gene, and practicability of its use.

To confirm the efficiency of gene therapy DNA vector VTvaf17-ANG carrying the therapeutic gene, namely the ANG gene, and practicability of its use, changes in angiogenin concentration in the cultural medium of HDFa human dermal fibroblast cells (ATCC PCS-20102) were assessed after transfection of these cells with gene therapy DNA vector VTvaf17-ANG carrying the human ANG gene, as described in Example 12.

HDFa human primary dermal fibroblast cells (ATCC PCS-2 grown as described in Example 12 were used to assess changes in angiogenin concentration.

After transfection, 0.1 ml of 1N HCl were added to 0.5 ml of the culture broth, mixed thoroughly, and incubated for 10 minutes at room temperature. Then, the mixture was neutralised by adding 0.1 ml of 1.2N NaOH/0.5M HEPES (pH 7-7.6) and stirred thoroughly. Supernatant was collected and used to assay the therapeutic protein.

The product of cDNA of ANG gene was assayed by enzyme-linked immunosorbent assay (ELISA) using ANG Human ELISA Kit (Abcam, USA) according to the manufacturer's method http://www.abcam.com/ps/products/99/ab99970/documents/ab99970 Angiogenin%20(ANG)%20Human%20ELISA_Kit%20v3%20(website).pdf. Optical density of the samples was measured at 450 nm using ChemWell Automated EIA and Chemistry Analyzer (Awareness Technology Inc., USA), and it is proportionate to ANG protein concentration in the sample. To measure the numerical value of concentration, the calibration curve constructed using calibrators from the kit with known concentrations of ANG protein was used. Variations between the calibrator optical density (OD) values in three replications did not exceed 10%.

R-3.0.2 was used for the statistical treatment of the results and data visualization (https://www.r-project.org/).

The diagram resulting from the assay is presented in FIG. 13 that indicates that the transfection of HDFa human primary dermal fibroblast cells (ATCCPCS-201-012 with gene therapy DNA vector VTvaf17-ANG carrying the ANG gene results in an increase of angiogenin concentration compared to reference samples, which indicates the efficiency of gene therapy DNA vector VTvaf17-ANG and confirms the ability of the vector to penetrate eukaryotic cells and express the ANG gene at the protein level. The presented results also confirm the practicability of use of gene therapy DNA vector VTvaf17-ANG in order to increase the expression level of ANG gene in eukaryotic cells.

### Example 24

Proof of the efficiency of gene therapy DNA vector VTvaf17-ANGPT1 carrying the therapeutic gene, namely the ANGPT1 gene, and practicability of its use.

To confirm the efficiency of gene therapy DNA vector VTvaf17-ANGPT1 carrying the therapeutic gene, namely the ANGPT1 gene, and practicability of its use, changes in angiopoietin 1 concentration in the cultural medium of HT 297.T human dermal fibroblast cells (ATCC® CRL-7782™) were assessed after transfection of these cells with gene therapy DNA vector VTvaf17-ANGPT1 carrying the human ANGPT1 gene, as described in Example 13.

HT 297.T human dermal fibroblast cells grown as described in Example 13 were used to assess changes in angiopoietin 1 concentration.

The product of cDNA of ANGPT1 gene was assayed by enzyme-linked immunosorbent assay (ELISA) using ANGPT1 Human ELISA Kit (Abcam, USA) according to the manufacturer's method https://www.abcam.com/ps/products/99/ab99972/documents/ab99972 Angiopoietin%201%20(ANG1)%20Human%20ELISA_Kit%20v%204%20(website).pdf

Optical density of the samples was measured at 450 nm using ChemWell Automated EIA and Chemistry Analyzer (Awareness Technology Inc., USA), and it is proportionate to ANGPT1 protein concentration in the sample. To measure the numerical value of concentration, the calibration curve constructed using calibrators from the kit with known concentrations of ANGPT1 protein was used. Variations between the calibrator optical density (OD) values in three replications did not exceed 10%.

R-3.0.2 was used for the statistical treatment of the results and data visualization (https://www.r-project.org/).

The diagram resulting from the assay is presented in FIG. 14 that indicates that the transfection of HT 297.T human dermal fibroblast cells with gene therapy DNA vector VTvaf17-ANGPT1 carrying the ANGPT1 gene results in an increase of angiopoietin concentration compared to reference samples, which indicates the efficiency of gene therapy DNA vector VTvaf17-ANGPT1 and confirms the ability of the vector to penetrate eukaryotic cells and express the ANGPT1 gene at the protein level. The presented results also confirm the practicability of use of gene therapy DNA vector VTvaf17-ANGPT1 in order to increase the expression level of ANGPT1 gene in eukaryotic cells.

### Example 25

Proof of the efficiency of gene therapy DNA vector VTvaf17-VEGFA carrying the therapeutic gene, namely the VEGFA gene, and practicability of its use.

To confirm the efficiency of gene therapy DNA vector VTvaf17-VEGFA carrying the therapeutic gene, namely the VEGFA gene, and practicability of its use, changes in the vascular endothelial growth factor concentration in the culture medium of Hs27 primary foreskin fibroblast cells (ATCC® CRL-1634™) were assessed after transfection of these cells with gene therapy DNA vector VTvaf17-VEGFA carrying the human VEGFA gene, as described in Example 14.

Hs27 primary foreskin fibroblast cells (ATCC® CRL-1634™) grown as described in Example 14 were used to assess changes in the vascular endothelial growth factor concentration.

The product of cDNA of VEGFA gene was assayed by enzyme-linked immunosorbent assay (ELISA) using VEGFA Human ELISA Kit (Abcam, USA) according to the manufacturer's method https://www.abcam.com/ps/products/1.19/ab119566/documents/ab119566%20-%20VEGFA%20Human%20ELISA%20Kit%20v5%20(website).pdf Optical density of the samples was measured at 450 nm using ChemWell Automated EIA and Chemistry Analyzer (Awareness Technology Inc., USA), and it is proportionate to VEGFA protein concentration in the sample. To measure the numerical value of concentration, the calibration curve constructed using calibrators from the kit with known concentrations of VEGFA protein was used. Variations between the calibrator optical density (OD) values in three replications did not exceed 10%.

R-3.0.2 was used for the statistical treatment of the results and data visualization (https://www.r-project.org/).

The diagram resulting from the assay is presented in FIG. 15 that indicates that the transfection of Hs27 primary foreskin fibroblast cells with gene therapy DNA vector VTvaf17-VEGFA carrying the VEGFA gene results in an increase of vascular endothelial growth factor concentration compared to reference samples, which indicates the efficiency of gene therapy DNA vector VTvaf17-VEGFA and confirms the ability of the vector to penetrate eukaryotic cells and express the VEGFA gene at the protein level. The presented results also confirm the practicability of use of gene therapy DNA vector VTvaf17-VEGFA in order to increase the expression level of VEGFA gene in eukaryotic cells.

### Example 26

Proof of the efficiency of gene therapy DNA vector VTvaf17-FGF1 carrying the therapeutic gene, namely the FGF1 gene, and practicability of its use.

To confirm the efficiency of gene therapy DNA vector VTvaf17-FGF1 carrying the therapeutic gene, namely the FGF1 gene, and practicability of its use, changes in the fibroblast growth factor 1 concentration in the culture medium of HSkM human primary skeletal muscle myoblast cells (A12555, Thermo Fisher Scientific) were assessed after transfection of these cells with gene therapy DNA vector VTvaf17-FGF1 carrying the human FGF1 gene, as described in Example 15.

HSkM human skeletal muscle myoblast cells (A12555, Thermo Fisher Scientific) grown as described in Example 15 were used to assess changes in the fibroblast growth factor 1 concentration.

The product of cDNA of FGF1 gene was assayed by enzyme-linked immunosorbent assay (ELISA) using FGF1 Human ELISA Kit (Abcam, USA) according to the manufacturer's method https://www.abcam.com/ps/products/219/ab219636/documents/ab219636_Hu%20F GF1_31%20Mar%202017%20(website).pdf Optical density of the samples was measured at 450 nm using ChemWell Automated EIA and Chemistry Analyzer (Awareness Technology Inc., USA), and it is proportionate to FGF1 protein concentration in the sample. To measure the numerical value of concentration, the calibration curve constructed using calibrators from the kit with known concentrations of FGF1 protein was used. Variations between the calibrator optical density (OD) values in three replications did not exceed 10%.

R-3.0.2 was used for the statistical treatment of the results and data visualization (https://www.r-project.org/).

The diagram resulting from the assay is presented in FIG. 16 that indicates that the transfection of HSkM human primary skeletal muscle myoblast cells with gene therapy DNA vector VTvaf17-FGF1 carrying the FGF1 gene results in an increase of fibroblast growth factor 1 concentration compared to reference samples, which indicates the efficiency of gene therapy DNA vector VTvaf17-FGF1 and confirms the ability of the vector to penetrate eukaryotic cells and express the FGF1 gene at the protein level. The presented results also confirm the practicability of use of gene therapy DNA vector VTvaf17-FGF1 in order to increase the expression level of FGF1 gene in eukaryotic cells.

### Example 27

Proof of the efficiency of gene therapy DNA vector VTvaf17-HIF1α carrying the therapeutic gene, namely the HIF1α gene, and practicability of its use.

To confirm the efficiency of gene therapy DNA vector VTvaf17-HIF1α carrying the therapeutic gene, namely the HIF1α gene, and practicability of its use, changes in the hypoxia-inducible factor concentration in the culture medium of HBdSMc human primary urinary bladder smooth muscle cells (ATCC® PCS-420-012™) were assessed after transfection of these cells with gene therapy DNA vector VTvaf17-HIF1α carrying the human HIF1α gene, as described in Example 16.

HBdSMc human primary urinary bladder smooth muscle cells grown as described in Example 16 were used to assess changes in the hypoxia-inducible factor alpha concentration.

The product of cDNA of HIF1α gene was assayed by enzyme-linked immunosorbent assay (ELISA) using Human HIF1alpha ELISA Kit (Abcam, USA) according to the manufacturer's method https://www.abcam.com/ps/products/171/ab171577/documents/ab171577_HIF1α_20.180116_ACW%20(website).pdf

Optical density of the samples was measured at 450 nm using ChemWell Automated EIA and Chemistry Analyzer (Awareness Technology Inc., USA), and it is proportionate to HIF1α protein concentration in the sample. To measure the numerical value of concentration, the calibration curve constructed using calibrators from the kit with known concentrations of HIF1α protein was used. Variations between the calibrator optical density (OD) values in three replications did not exceed 10%.

R-3.0.2 was used for the statistical treatment of the results and data visualization (https://www.r-project.org/).

The diagram resulting from the assay is presented in FIG. 17 that indicates that the transfection of HBdSMc human primary urinary bladder smooth muscle cells with gene therapy DNA vector VTvaf17-HIF1α carrying the HIF1α gene results in an increase of hypoxia-inducible factor alpha concentration compared to reference samples, which indicates the efficiency of gene therapy DNA vector VTvaf17-HIF1α and confirms the ability of the vector to penetrate eukaryotic cells and express the HIF1α gene at the protein level. The presented results also confirm the practicability of use of gene therapy DNA vector VTvaf17-HIF1α in order to increase the expression level of HIF1α gene in eukaryotic cells.

### Example 28

Proof of the efficiency of gene therapy DNA vector VTvaf17-HGF carrying the therapeutic gene, namely the HGF gene, and practicability of its use.

To confirm the efficiency of gene therapy DNA vector VTvaf17-HGF carrying the therapeutic gene, namely the HGF gene, and practicability of its use, changes in the hepatocyte growth factor concentration in the cultural medium of T/GHA-VSMC aortic smooth muscle cells (ATCC® CRL-1999™) were assessed after transfection of these cells with gene therapy DNA vector VTvaf17-HGF carrying the human HGF gene, as described in Example 17.

T/GHA-VSMC primary aortic smooth muscle cells grown as described in Example 17 were used to assess changes in the hepatocyte growth factor concentration.

The product of cDNA of HGF gene was assayed by enzyme-linked immunosorbent assay (ELISA) using Human HGF ELISA Kit (Abcam, USA) according to the manufacturer's method https://www.abcam.com/ps/products/100/ab100534/documents/ab100534%20HGF % 20Human%20ELISA_Kit%20v3%20(website).pdf

Optical density of the samples was measured at 450 nm using ChemWell Automated EIA and Chemistry Analyzer (Awareness Technology Inc., USA), and it is proportionate to HGF protein concentration in the sample. To measure the numerical value of concentration, the calibration curve constructed using calibrators from the kit with known concentrations of HGF protein was used. Variations between the calibrator optical density (OD) values in three replications did not exceed 10%.

R-3.0.2 was used for the statistical treatment of the results and data visualization (https://www.r-project.org/).

The diagram resulting from the assay is presented in FIG. 18 that indicates that the transfection of T/GHA-VSMC primary aortic smooth muscle cells with gene therapy DNA vector VTvaf17-HGF carrying the HGF gene results in an increase of hepatocyte growth factor concentration compared to reference samples, which indicates the efficiency of gene therapy DNA vector VTvaf17-HGF and confirms the ability of the vector to penetrate eukaryotic cells and express the HGF gene at the protein level. The presented results also confirm the practicability of use of gene therapy DNA vector VTvaf17-HGF in order to increase the expression level of HGF gene in eukaryotic cells.

### Example 29

Proof of the efficiency of gene therapy DNA vector VTvaf17-SDF1 carrying the therapeutic gene, namely the SDF1 gene, and practicability of its use.

To confirm the efficiency of gene therapy DNA vector VTvaf17-SDF1 carrying the therapeutic gene, namely the SDF1 gene, and practicability of its use, changes in the stromal cell-derived factor 1 concentration in the culture medium of HEKa primary epidermal keratinocyte cells (ATCC® PCS-200-011™) were assessed after transfection of these cells with gene therapy DNA vector VTvaf17-SDF1 carrying the human SDF1 gene, as described in Example 18.

HEKa primary epidermal keratinocyte cells grown as described in Example 18 were used to assess changes in the stromal cell-derived factor 1 concentration.

The product of cDNA of SDF1 gene was assayed by enzyme-linked immunosorbent assay (ELISA) using Human SDF1 ELISA Kit (Abcam, USA) according to the manufacturer's method https://www.abcam.com/ps/products/100/ab100637/documents/ab100637%20SDF1%20alpha%20Human%20ELISA_Kit%20v4%20(website).pdf

Optical density of the samples was measured at 450 nm using ChemWell Automated EIA and Chemistry Analyzer (Awareness Technology Inc., USA), and it is proportionate to SDF1 protein concentration in the sample. To measure the numerical value of concentration, the calibration curve constructed using calibrators from the kit with known concentrations of SDF1 protein was used. Variations between the calibrator optical density (OD) values in three replications did not exceed 10%.

R-3.0.2 was used for the statistical treatment of the results and data visualization (https://www.r-project.org/).

The diagram resulting from the assay is presented in FIG. 19 that indicates that the transfection of HEKa primary epidermal keratinocyte cells with gene therapy DNA vector VTvaf17-SDF1 carrying the SDF1 gene results in an increase of stromal cell-derived factor 1 concentration compared to reference samples, which indicates the efficiency of gene therapy DNA vector VTvaf17-SDF1 and confirms the ability of the vector to penetrate eukaryotic cells and express the SDF1 gene at the protein level. The presented results also confirm the practicability of use of gene therapy DNA vector VTvaf17-SDF1 in order to increase the expression level of SDF1 gene in eukaryotic cells.

### Example 30

Proof of the efficiency of gene therapy DNA vector VTvaf17-KLK4 carrying the therapeutic gene, namely the KLK4 gene, and practicability of its use.

To confirm the efficiency of gene therapy DNA vector VTvaf17-KLK4 carrying the therapeutic gene, namely the KLK4 gene, and practicability of its use, changes in the kallikrein concentration in the culture medium of HUVEC primary umbilical vein endothelial cells (ATCC® PCS-100-010™) were assessed after transfection of these cells with gene therapy DNA vector VTvaf17-KLK4 carrying the human KLK4 gene, as described in Example 19.

HUVEC primary umbilical vein endothelial cells grown as described in Example 19 were used to assess changes in the kallikrein concentration.

The product of cDNA of KLK4 gene was assayed by enzyme-linked immunosorbent assay (ELISA) using Human kallikrein-related peptidase 4 (KLK4) ELISA Kit (MyBioSource, USA) according to the manufacturer's method https://www.mybiosource.com/prods/ELISA-Kit/Human/kallikrein-related-peptidase-4/KLK4/datasheet.php?products_id=917102 Optical density of the samples was measured at 450 nm using ChemWell Automated EIA and Chemistry Analyzer (Awareness Technology Inc., USA), and it is proportionate to KLK4 protein concentration in the sample. To measure the numerical value of concentration, the calibration curve constructed using calibrators from the kit with known concentrations of KLK4 protein was used. Variations between the calibrator optical density (OD) values in three replications did not exceed 10%.

R-3.0.2 was used for the statistical treatment of the results and data visualization (https://www.r-project.org/).

The diagram resulting from the assay is presented in FIG. 20 that indicates that the transfection of HUVEC primary umbilical vein endothelial cells with gene therapy DNA vector VTvaf17-KLK4 carrying the KLK4 gene results in an increase of kallikrein concentration compared to reference samples, which indicates the efficiency of gene therapy DNA vector VTvaf17-KLK4 and confirms the ability of the vector to penetrate eukaryotic cells and express the KLK4 gene at the protein level. The presented results also confirm the practicability of use of gene therapy DNA vector VTvaf17-KLK4 in order to increase the expression level of KLK4 gene in eukaryotic cells.

### Example 31

Proof of the efficiency of gene therapy DNA vector VTvaf17-PDGFC carrying the therapeutic gene, namely the PDGFC gene, and practicability of its use.

To confirm the efficiency of gene therapy DNA vector VTvaf17-PDGFC carrying the therapeutic gene, namely the PDGFC gene, and practicability of its use, changes in the platelet growth factor C concentration in the culture medium of HEMa primary epidermal melanocyte cells (ATCC® PCS-200-013™) were assessed after transfection of these cells with gene therapy DNA vector VTvaf17-PDGFC carrying the human PDGFC gene, as described in Example 20.

HEMa primary epidermal melanocyte cells (ATCC® PCS-200-013™) grown as described in Example 20 were used to assess changes in the platelet growth factor C concentration.

The product of cDNA of PDGFC gene was assayed by enzyme-linked immunosorbent assay (ELISA) using Human PDGFC ELISA Kit (MyBioSource, USA) according to the manufacturer's method https://www.mybiosource.com/prods/ELISA-Kit/Human/PDGFC/datasheet.php?products_id=2501938

Optical density of the samples was measured at 450 nm using ChemWell Automated EIA and Chemistry Analyzer (Awareness Technology Inc., USA), and it is proportionate to PDGFC protein concentration in the sample.

To measure the numerical value of concentration, the calibration curve constructed using calibrators with known concentrations of protein was used with detection of the optical density at 450 nm wavelength using ChemWell Automated EIA and Chemistry Analyzer (Awareness Technology Inc., USA).

To measure the numerical value of concentration, the calibration curve constructed using the reference samples from the kit with known concentrations of PDGFC protein was used. Variations between the calibrator optical density (OD) values in three replications did not exceed 10%.

R-3.0.2 was used for the statistical treatment of the results and data visualization (https://www.r-project.org/).

The diagram resulting from the assay is presented in FIG. 21 that indicates that the transfection of HEMa primary epidermal melanocyte cells (ATCC® PCS-200-013™) with gene therapy DNA vector VTvaf17-PDGFC carrying the PDGFC gene results in an increase of platelet growth factor C concentration compared to reference samples, which indicates the efficiency of gene therapy DNA vector VTvaf17-PDGFC and confirms the ability of the vector to penetrate eukaryotic cells and express the PDGFC gene at the protein level. The presented results also confirm the practicability of use of gene therapy DNA vector VTvaf17-PDGFC in order to increase the expression level of PDGFC gene in eukaryotic cells.

### Example 32

Proof of the efficiency of gene therapy DNA vector VTvaf17-PROK1 carrying the therapeutic gene, namely the PROK1 gene, and practicability of its use.

To confirm the efficiency of gene therapy DNA vector VTvaf17-PROK1 carrying the therapeutic gene, namely the PROK1 gene, and practicability of its use, changes in the prokineticin-1 concentration in the culture medium of HSkM human primary skeletal muscle myoblast cells (A12555, Thermo Fisher Scientific) were assessed after transfection of these cells with gene therapy DNA vector VTvaf17-PROK1 carrying the human PROK1 gene, as described in Example 21.

HSkM human skeletal muscle myoblast cells (A12555, Thermo Fisher Scientific) grown as described in Example 21 were used to assess changes in the prokineticin-1 concentration.

The product of cDNA of PROK1 gene was assayed by enzyme-linked immunosorbent assay (ELISA) using the PROK1 elisa kit: Human EG-VEGF ELISA Kit (MyBioSource, USA) according to the manufacturer's method https://www.mybiosource.com/images/tds/protocol_manuals/000000-799999/MBS175861.pdf Optical density of the samples was measured at 450 nm using ChemWell Automated EIA and Chemistry Analyzer (Awareness Technology Inc., USA), and it is proportionate to PROK1 protein concentration in the sample. To measure the numerical value of concentration, the calibration curve constructed using calibrators from the kit with known concentrations of PROK1 protein was used. Variations between the calibrator optical density (OD) values in three replications did not exceed 10%.

R-3.0.2 was used for the statistical treatment of the results and data visualization (https://www.r-project.org/).

The diagram resulting from the assay is presented in FIG. 22 that indicates that the transfection of human primary skeletal muscle myoblast cells HSkM with gene therapy DNA vector VTvaf17-PROK1 carrying the PROK1 gene results in an increase of prokineticin-1 protein concentration compared to reference samples, which indicates the efficiency of gene therapy DNA vector VTvaf17-PROK1 and confirms the ability of the vector to penetrate eukaryotic cells and express the PROK1 gene at the protein level. The presented results also confirm the practicability of use of gene therapy DNA vector VTvaf17-PROK1 in order to increase the expression level of PROK1 gene in eukaryotic cells.

### Example 33

Proof of the efficiency of gene therapy DNA vector VTvaf17-PROK2 carrying the therapeutic gene, namely the PROK2 gene, and practicability of its use.

To confirm the efficiency of gene therapy DNA vector VTvaf17-PROK2 carrying the therapeutic gene, namely the PROK2 gene, and practicability of its use, changes in prokineticin-2 concentration in the cultural medium of HMEC-1 dermal microvascular endothelial cells (ATCC® CRL-3243™) were assessed after transfection of these cells with gene therapy DNA vector VTvaf17-PROK2 carrying the human PROK2 gene, as described in Example 22.

HMEC-1 primary dermal microvascular endothelial cells (ATCC® CRL-3243™) grown as described in Example 22 were used to assess changes in the prokineticin-2 concentration.

The product of cDNA of PROK2 gene was assayed by enzyme-linked immunosorbent assay (ELISA) using Human Prokineticin-2, PROK2 ELISA Kit (MyBioSource, USA) according to the manufacturer's method https://www.mybiosource.com/images/tds/protocol_manuals/800000-9999999/MBS940962.pdf Optical density of the samples was measured at 450 nm wavelength using ChemWell Automated EIA and Chemistry Analyzer (Awareness Technology Inc., USA), and it is proportionate to PROK2 protein concentration in the sample. To measure the numerical value of concentration, the calibration curve constructed using calibrators from the kit with known concentrations of PROK2 protein was used. Variations between the calibrator optical density (OD) values in three replications did not exceed 10%.

R-3.0.2 was used for the statistical treatment of the results and data visualization (https://www.r-project.org/).

The diagram resulting from the assay is presented in FIG. 23 that indicates that the transfection of human dermal microvascular endothelial cells HMEC-1 (ATCC® CRL-3243™) with gene therapy DNA vector VTvaf17-PROK2 carrying the PROK2 gene results in an increase of prokineticin-2 protein concentration compared to reference samples, which indicates the efficiency of gene therapy DNA vector VTvaf17-PROK2 and confirms the ability of the vector to penetrate eukaryotic cells and express the PROK2 gene at the protein level. The presented results also confirm the practicability of use of gene therapy DNA vector VTvaf17-PROK2 in order to increase the expression level of PROK2 gene in eukaryotic cells.

### Example 34

Proof of the efficiency and practicability of use of gene therapy DNA vector VTvaf17-ANG carrying the ANG gene in order to increase the expression of ANG protein in human tissues

To analyse changes in the angiogenin protein concentration, gene therapy DNA vector VTvaf17-ANG carrying the ANG gene was injected into the forearm skin of three patients with concurrent injection of a placebo being gene therapy DNA vector VTvaf17 devoid of the ANG gene.

Patient 1, man, 64 y.o. (P1); Patient 2, woman, 66 y.o. (P2); Patient 3, man, 62 y.o. (P3). Polyethyleneimine Transfection reagent cGMP grade in-vivo-jetPEI (Polyplus Transfection, France) was used as a transport system. Gene therapy DNA vector VTvaf17-ANG containing the ANG gene and gene therapy DNA vector VTvaf17 used as a placebo were dissolved in sterile nuclease-free water. DNA-cGMP grade in-vivo-jetPEI complexes were prepared according to the manufacturer recommendations.

Gene therapy DNA vector VTvaf17 (placebo) and gene therapy DNA vector VTvaf17-ANG were injected in the quantity of 1 mg for each genetic construct using the tunnel method with a 30 G needle to the depth of 3 mm. The injectate volume of gene therapy DNA vector VTvaf17 (placebo) and gene therapy DNA vector VTvaf17-ANG was 0.3 ml for each genetic construct. The points of injection of each genetic construct were located at 8 to 10 cm intervals at the forearm site.

The biopsy samples were taken on the 2nd day after the injection of the genetic constructs of gene therapy DNA vectors. The biopsy samples were taken from the patients' skin in the site of injection of gene therapy DNA vector VTvaf17-ANG carrying the ANG gene (I), gene therapy DNA vector VTvaf17 (placebo) (II), and from intact skin (III) using the skin biopsy device Epitheasy 3.5 (Medax SRL, Italy). The skin of patients in the biopsy site was preliminarily rinsed with sterile saline and anaesthetised with a lidocaine solution. The biopsy sample size was ca. 10 mm3, and the weight was approximately 11 mg. The sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used to assay the therapeutic angiogenin protein by enzyme-linked immunosorbent assay (ELISA) using the ANG Human ELISA Kit (Abcam, USA) as described in Example 23 with optical density detection at 450 nm wavelength using ChemWell Automated EIA and Chemistry Analyser (Awareness Technology Inc., USA).

To measure the numerical value of concentration, the calibration curve constructed using the reference samples from the kit with known concentrations of the angiogenin protein was used. Diagrams resulting from the assay are shown in FIG. 24.

FIG. 24 shows an increase in the concentration of angiogenin protein in the skin of all three patients in the injection site of gene therapy DNA vector VTvaf17-ANG carrying the human ANG therapeutic gene compared to the concentration of angiogenin protein in the injection site of gene therapy DNA vector VTvaf17 (placebo) devoid of the human ANG gene, which indicates the efficiency of gene therapy DNA vector VTvaf17-ANG and confirms the practicability of its use, in particular upon injection of gene therapy DNA vector in human organs.

### Example 35

Proof of the efficiency of gene therapy DNA vector VTvaf17-ANGPT1 carrying the ANGPT1 gene and practicability of its use in order to increase the expression level of the angiopoietin protein in human organs by introducing autologous fibroblasts transfected with gene therapy DNA vector VTvaf17-ANGPT1.

To confirm the efficiency of gene therapy DNA vector VTvaf17-ANGPT1 carrying the ANGPT1 gene and practicability of its use, changes in the angiopoietin protein concentration in human skin upon injection of patient's skin with autologous fibroblast culture of the same patient transfected with gene therapy DNA vector VTvaf17-ANGPT1 were assessed.

The appropriate autologous fibroblast culture transfected with the gene therapy DNA vector VTvaf17-ANGPT1 carrying the ANGPT1 gene was injected into the patient's forearm skin with concurrent injection of a placebo in the form of autologous fibroblast culture transfected with gene therapy DNA vector VTvaf17 not carrying the ANGPT1 gene.

The human primary fibroblast culture was isolated from the patient skin biopsy specimens. Biopsy specimens of the skin from the area protected by ultraviolet, namely behind the ear or on the inner lateral side of the elbow, were taken using the skin biopsy device Epitheasy 3.5 (Medax SRL, Italy). The biopsy sample was ca. 10 mm and ca. 11 mg. The patient's skin was preliminarily rinsed with sterile saline and anaesthetised with a lidocaine solution. The primary cell culture was cultivated at 37° C. in the presence of 5% CO2, in the DMEM medium with 10% fetal bovine serum and 100 U/ml of ampicillin. The passage and change of culture medium was performed every 2 days. Total duration of culture growth did not exceed 25-30 days. Then an aliquot of 5×10⁴ cells was taken from the cell culture. The patient's fibroblast culture was transfected with the gene therapy DNA vector VTvaf17-ANGPT1 carrying the ANGPT1 gene or placebo, i.e. VTvaf17 vector not carrying the ANGPT1 therapeutic gene.

The transfection was carried out using a cationic polymer such as polyethyleneimine JETPEI (Polyplus transfection, France), according to the manufacturer's instructions. The cells were cultured for 72 hours and then injected into the patient. Injection of autologous fibroblast culture of the patient transfected with gene therapy DNA vector VTvaf17-ANGPT1, and autologous fibroblast culture of the patient transfected with gene therapy DNA vector VTvaf17 as a placebo was performed in the forearm using the tunnel method with a 13 mm long 30 G needle to the depth of approximately 3 mm. The concentration of the modified autologous fibroblasts in the injected suspension was approximately 5 mln cells per 1 ml of the suspension, the dose of the injected cells did not exceed 15 mln. The points of injection of the autologous fibroblast culture were located at 8 to 10 cm intervals.

Biopsy samples were taken on the 4th day after the injection of autologous fibroblast culture transfected with the gene therapy DNA vector VTvaf17-ANGPT1 carrying the therapeutic gene, namely ANGPT1 gene, and placebo. Biopsy was taken from the patient's skin in the site of injection of autologous fibroblast culture transfected with gene therapy DNA vector VTvaf17-ANGPT1 carrying the ANGPT1 therapeutic gene (C), autologous fibroblast culture transfected with gene therapy DNA vector VTvaf17 not carrying the ANGPT1 therapeutic gene (placebo) (B), as well as from intact skin site (A) using the skin biopsy device Epitheasy 3.5 (Medax SRL, Italy), and then procedures were performed as described in Example 34.

The angiopoietin protein concentration was assayed in the supernatants of patient's skin biopsy samples by enzyme-linked immunosorbent assay (ELISA) using the ANGPT1 Human ELISA Kit (Abcam, USA) according to the manufacturer's method (see Example 24) with optical density detection at 450 nm wavelength using ChemWell Automated EIA and Chemistry Analyser (Awareness Technology Inc., USA).

To measure the numerical value of concentration, the calibration curve constructed using the reference samples from the kit with known concentrations of the angiopoietin protein was used. Diagrams resulting from the assay are shown in FIG. 25.

FIG. 25 shows an increase in the concentration of angiopoietin protein in the area of the patient's skin in the injection site of autologous fibroblast culture transfected with the gene therapy DNA vector VTvaf17-ANGPT1 carrying the ANGPT1 gene compared to the same protein concentration in the injection site of autologous fibroblast culture transfected with the gene therapy DNA vector VTvaf17 that does not carry the ANGPT1 gene (placebo), which indicates the efficiency of gene therapy DNA vector VTvaf17-ANGPT1 and practicability of its use in order to increase the expression level of ANGPT1 in human organs, in particular upon injection of autologous fibroblasts transfected with the gene therapy DNA vector VTvaf17-ANGPT1 into the skin.

### Example 36

Proof of the efficiency and practicability of use of gene therapy DNA vector VTvaf17-SDF1 carrying the SDF1 gene in order to increase the expression of stromal cell-derived factor in human tissues.

Gene therapy DNA vector VTvaf17-SDF1 was injected with concurrent injection of a placebo being vector plasmid VTvaf17 devoid of the cDNA of SDF1 gene into the muscle tissue of the patient in the forearm site in order to analyse the expression level of the SDF1 therapeutic gene.

Polyethyleneimine Transfection reagent cGMP grade in-vivo-jetPEI (Polyplus Transfection, France) was used as a transport system. Gene therapy DNA vector VTvaf17-SDF1 containing SDF1 gene and gene therapy DNA vector VTvaf17 used as a placebo were dissolved in sterile nuclease-free water. DNA-cGMP grade in-vivo-jetPEI complexes were prepared according to the manufacturer recommendations.

The resulting complexes were used for injection of the patient. The injection was made using the tunnel method with a 30 G needle to the depth of 15 to 20 mm. The solution of gene therapy DNA vector VTvaf17-SDF1 and placebo was introduced in the volume of ca. 0.5 ml each. The points of injection of DNA vector and placebo were located at 5 to 7 cm intervals.

Biopsy samples were taken on the 3^{rd} day after the injection of the gene therapy substance. Biopsy was taken from the muscle tissue areas in the site of injection of gene therapy VTvaf17-SDF1 (P1I), as well as from intact muscle areas (P1III) and the site of placebo injection (P1II) using the automatic biopsy sampler MAGNUM (BARD, USA), and then procedures were performed as described in Example 34.

Stromal cell-derived factor protein was assayed in the lysates of the patient's muscle tissue biopsies by enzyme-linked immunosorbent assay (ELISA) using Human SDF1 ELISA Kit (Abcam, USA) as described in Example 29.

Diagrams resulting from the assay are shown in FIG. 26.

It was shown that the level of stromal cell-derived factor protein was increased in the muscle tissue of the patient in the area of injection of gene therapy DNA vector VTvaf17-SDF1 with cDNA of SDF1 gene. Whereas level of stromal cell-derived factor protein in muscle tissue did not change after placebo administration, which indicates the enhanced expression of SDF1 gene when gene therapy DNA vector VTvaf17-SDF1 is used. This also indicates the efficiency of gene therapy DNA vector VTvaf17-SDF1 and confirms the practicability of its use, in particular upon injection of the gene therapy DNA vector into human tissues.

### Example 37

Proof of the efficiency and practicability of combined use of gene therapy DNA vector VTvaf17-ANG carrying the ANG therapeutic gene, gene therapy DNA vector VTvaf17-VEGFA carrying the VEGFA therapeutic gene, gene therapy DNA vector VTvaf17-FGF1 carrying the FGF1 therapeutic gene, gene therapy DNA vector VTvaf17-PROK1 carrying the PROK1 therapeutic gene in order to increase the expression level of ANG, VEGFA, FGF1, and PROK1 proteins in mammalian tissues/organs.

To confirm the efficiency of gene therapy DNA vector VTvaf17-ANG carrying the ANG therapeutic gene, gene therapy DNA vector VTvaf17-VEGFA carrying the VEGFA therapeutic gene, gene therapy DNA vector VTvaf17-FGF1 carrying the FGF1 therapeutic gene, gene therapy DNA VTvaf17-PROK1 carrying the PROK1 therapeutic gene and practicability of combined use of these vectors, the change in the level of the following proteins: angiogenin, vascular endothelial growth factor A, fibroblast growth factor 1, prokineticin-1, respectively, was assessed in the intracutaneous injection sites of Wistar rats (male, 22-24 weeks old).

A mixture of gene therapy DNA vectors was prepared at the ratio of 1:1:1:1 (by weight) from lyophilisate of DNA vectors VTvaf17-ANG, VTvaf17-VEGFA, VTvaf17-FGF1, VTvaf17-PROK1 by dissolving in sterile nuclease-free water. Polyethyleneimine Transfection reagent cGMP grade in-vivo-jetPEI (Polyplus Transfection, France) was used as a transport system. DNA-cGMP grade in-vivo-jetPEI complexes were prepared according to the manufacturer recommendations.

3 groups, 11 animals each were formed. Intracutaneous injections were made to all animals under anaesthesia:
in group 1 (KI) using a mixture of gene therapy DNA vectors VTvaf17-ANG, VTvaf17-VEGFA, VTvaf17-FGF1, VTvaf17-PROK1 in a volume of 150 μl (the concentration of each gene therapy DNA vector is 1 μg/μl),
in group 2 (KII) using solution of DNA vector VTvaf17 in a volume of 150 μl with a concentration of DNA vector of 1 μg/μl, (placebo),
in group 3 (KIII) using saline solution in a volume of 150 μl.

The biopsy samples were taken 72 hours after the injection of the mixture of gene therapy DNA vectors and placebo. Biopsy was taken after necropsy of animals in the sites of injection of a mixture of four gene therapy DNA vectors carrying the ANG, VEGFA, FGF1, and PROK1 therapeutic genes (group 1), in the region of injection of solution of gene therapy DNA vector VTvaf17 (group 2), in the region of injection of saline solution (group 3). Mass of each biopsy sample was about 20 mg. Then manipulations with the obtained samples were performed as described in Example 34.

ANG, VEGFA, FGF1, PROK1 gene products were assayed by enzyme-linked immunosorbent assay (ELISA) using the ANG Human ELISA Kit (Abcam, USA), VEGFA Human ELISA Kit (Abcam, USA), FGF1 Human ELISA Kit (Abcam, USA), PROK1 elisa kit: Human EG-VEGF ELISA Kit (MyBioSource, USA). Preparation of test samples, measurement, and processing of results were performed as described in Examples 23, 25, 26, and 32.

Diagrams resulting from the assay are shown in FIG. 27 that shows that in the injured area of animals in the injection site of a mixture of four gene therapy DNA vectors: VTvaf17-ANG, VTvaf17-VEGFA, VTvaf17-FGF1, and VTvaf17-PROK1 in group 1 of animals the level of the following proteins: angiogenin protein, vascular endothelial growth factor A protein, fibroblast growth factor 1 protein, and prokineticin-1 protein was significantly increased compared to the level of angiogenin, vascular endothelial growth factor A, fibroblast growth factor 1, and prokineticin-1 in groups 2 and 3.

The presented results confirm the practicability of use of gene therapy DNA vectors VTvaf17-ANG, VTvaf17-VEGFA, VTvaf17-FGF1, and VTvaf17-PROK1 and efficiency of their use in order to increase the expression level of proteins such as angiogenin, vascular endothelial growth factor A, fibroblast growth factor 1, prokineticin-1 in mammalian tissues/organs.

### Example 38

Proof of the efficiency and practicability of combined use of gene therapy DNA vector VTvaf17-ANG carrying the ANG therapeutic gene, gene therapy DNA vector VTvaf17-HIF1α carrying the HIF1α therapeutic gene, gene therapy DNA vector VTvaf17-PDGFC carrying the PDGFC therapeutic gene, gene therapy DNA vector VTvaf17-PROK2 carrying the PROK2 therapeutic gene in order to increase the expression level of ANG, HIF1α, PDGFC, and PROK2 proteins in human tissues.

To confirm the efficiency of gene therapy DNA vector VTvaf17-ANG carrying the ANG therapeutic gene, gene therapy DNA vector VTvaf17-HIF1α carrying the HIF1α therapeutic gene, gene therapy DNA vector VTvaf17-PDGFC carrying the PDGFC therapeutic gene, gene therapy DNA VTvaf17-PROK2 carrying the PROK2 therapeutic gene and practicability of combined use of these vectors, the change in the level of the following proteins: angiogenin, hypoxia-inducible factor, platelet growth factor C, prokineticin-2, respectively was assessed in the muscle tissue in the forearm site.

A mixture of gene therapy DNA vectors was prepared at the ratio of 1:1:1:1 (by weight) from lyophilisate of DNA vectors VTvaf17-ANG, VTvaf17-HIF1α, VTvaf17-PDGFC, and VTvaf17-PROK2 by dissolving in sterile nuclease-free water. The concentration of DNA vectors in the mixture was 1 mg/ml. Polyethyleneimine Transfection reagent cGMP grade in-vivo-jetPEI (Polyplus Transfection, France) was used as a transport system. DNA-cGMP grade in-vivo-jetPEI complexes were prepared according to the manufacturer recommendations. The gene therapy DNA vector VTvaf17 solution at a concentration of 1 mg/ml was used as a placebo.

The resulting mixture of DNA vectors VTvaf17-ANG, VTvaf17-HIF1α, VTvaf17-PDGFC, and VTvaf17-PROK2, as well as the placebo was used for injection of the patient using the tunnel method with a 30 G needle to the depth of 15 to 20 mm. The injectate volume of a mixture of DNA vectors and placebo was about 0.6 ml for each. The points of injection of a mixture of DNA vectors and the placebo were located at 7 to 8 cm intervals.

Biopsy samples were taken on the 3rd day after the introduction of a mixture of DNA vectors and the placebo. Biopsy was taken from the muscle tissue areas in the site of injection of gene therapy vectors VTvaf17-ANG, VTvaf17-HIF1α, VTvaf17-PDGFC, VTvaf17-PROK2 (P1I), as well as from intact muscle areas (P1III) and the area of placebo injection (P1II) using the automatic biopsy sampler MAGNUM (BARD, USA), and then procedures were performed as described in Example 34.

ANG, HIF1α, PDGFC, and PROK2 gene products were assayed by enzyme-linked immunosorbent assay (ELISA) using the ANG Human ELISA Kit (Abcam, USA), Human HIF1alpha ELISA Kit (Abcam, USA), Human PDGFC ELISA Kit (MyBioSource, USA), Human Prokineticin-2, PROK2 ELISA Kit (MyBioSource, USA): Measurement and processing of results were performed as described in Examples 23, 27, 31, 33.

Diagrams resulting from the assay are shown in FIG. 28 showing that in the region of injection of a mixture of four gene therapy DNA vectors: VTvaf17-ANG, VTvaf17-HIF1α, VTvaf17-PDGFC, and VTvaf17-PROK2, the concentration of the following proteins: angiogenin, hypoxia-inducible factor, platelet growth factor C, and prokineticin-2 was significantly increased compared to the concentration of these proteins in the region of placebo (DNA vector VTvaf17) injection.

The presented results confirm the practicability of use of gene therapy DNA vectors VTvaf17-ANG, VTvaf17-HIF1α, VTvaf17-PDGFC, and VTvaf17-PROK2 and efficiency of their use in order to increase the expression level of proteins such as angiogenin, hypoxia-inducible factor, platelet growth factor C, and prokineticin-2 in human tissues.

### Example 39

Proof of the efficiency of gene therapy DNA vector VTvaf17-HGF carrying the HGF gene and practicability of its use in order to increase the expression level of HGF protein in mammalian cells.

To confirm the efficiency of gene therapy DNA vector VTvaf17-HGF carrying the HGF gene, the change in mRNA accumulation of HGF therapeutic gene in BAOSMC bovine aortic smooth muscle cells (Genlantis) 48 hours after their transfection with gene therapy DNA vector VTvaf17-HGF carrying the human HGF gene were assessed compared to BEND reference cells transfected with gene therapy DNA vector VTvaf17 not carrying the human HGF gene (placebo).

BAOSMC bovine aortic smooth muscle cell culture (Genlantis) was grown in Bovine Smooth Muscle Cell Growth Medium (Sigma B311F-500) with the addition of bovine serum up to 10% (Paneco, Russia). Transfection with gene therapy DNA vector VTvaf17-HGF carrying the human HGF gene and DNA vector VTvaf17 not carrying the human HGF gene (reference), RNA extraction, reverse transcription reaction, PCR amplification, and data analysis were performed as described in Example 17. Bull/cow actin gene (ACT) listed in the GenBank database under number AH001130.2 was used as a reference gene. Positive control included amplicons from PCR on matrices represented by plasmids in known concentrations containing HGF and ACT gene sequences. Negative control included deionised water. Real-time quantification of the PCR products, i.e. HGF and ACT gene cDNAs obtained by amplification, was conducted using the Bio-Rad CFX Manager 2.1 software (Bio-Rad, USA).

Diagrams resulting from the assay are shown in FIG. 29.

FIG. 29 shows that the level of specific cDNA of human HGF gene has grown massively as a result of transfection of BAOSMC bovine aortic smooth muscle cells with gene therapy DNA vector VTvaf17-HGF, which confirms the ability of the vector to penetrate eukaryotic cells and express the HGF gene at the mRNA level. The presented results confirm the practicability of use of gene therapy DNA vector VTvaf17-HGF in order to increase the expression level of HGF gene in mammalian cells.

### Example 40

Escherichia coli strain SCS110-AF/VTvaf17-ANG, or Escherichia coli strain SCS110-AF/VTvaf17-ANGPT1, or Escherichia coli strain SCS110-AF/VTvaf17-VEGFA, or Escherichia coli strain SCS110-AF/VTvaf17-FGF1, or Escherichia coli strain SCS110-AF/VTvaf17-HIF1α, or Escherichia coli strain SCS110-AF/VTvaf17-HGF, or Escherichia coli strain SCS110-AF/VTvaf17-SDF1, or Escherichia coli strain SCS110-AF/VTvaf17-KLK4, or Escherichia coli strain SCS110-AF/VTvaf17-PDGFC, or Escherichia coli strain SCS110-AF/VTvaf17-PROK1, or Escherichia coli strain SCS110-AF/VTvaf17-PROK2 carrying the gene therapy DNA vector, method of its production.

The strain construction for the production of gene therapy DNA vector based on gene therapy DNA vector VTvaf17 carrying VTvaf17 the ANG, or ANGPT1, or VEGFA, or FGF1, or HIF1α, or HGF, or SDF1, or KLK4, or PDGFC, or PROK1, or PROK2 therapeutic genes on an industrial scale: namely Escherichia coli strain SCS10-AF/VTvaf17-ANG, or Escherichia coli strain SCS10-AF/VTvaf17-ANGPT1, or Escherichia coli strain SCS110-AF/VTvaf17-VEGFA, or Escherichia coli strain SCS10-AF/VTvaf17-FGF1, or Escherichia coli strain SCS110-AF/VTvaf17-HIF1α, or Escherichia coli strain SCS110-AF/VTvaf17-HGF, or Escherichia coli strain SCS110-AF VTvaf17-SDF1, or Escherichia coli strain SCS110-AF VTvaf17-KLK4, or Escherichia coli strain SCS110-AF VTvaf17-PDGFC, or Escherichia coli strain SCS110-AF/VTvaf17-PROK1, or Escherichia coli strain SCS110-AF/VTvaf17-PROK2 carrying gene therapy DNA vector VTvaf17-ANG, or VTvaf17-ANGPT1, or VTvaf17-VEGFA, or VTvaf17-FGF1, or VTvaf17-HIF1α, or VTvaf17-HGF, or VTvaf17-SDF1, or VTvaf17-KLK4, or VTvaf17-PDGFC, or VTvaf17-PROK1, or VTvaf17-PROK2, respectively, for its production allowing for antibiotic-free selection involves making electrocompetent cells of Escherichia coli strain SCS110-AF and subjecting these cells to electroporation with gene therapy DNA vector VTvaf17-ANG, or VTvaf17-ANGPT1, or VTvaf17-VEGFA, or VTvaf17-FGF1, or VTvaf17-HIF1α, or VTvaf17-HGF, or VTvaf17-SDF1, or VTvaf17-KLK4, or VTvaf17-PDGFC, or VTvaf17-PROK1, or VTvaf17-PROK2. After that, the cells were poured into agar plates (Petri dishes) with a selective medium containing yeastrel, peptone, 6% sucrose, and 10 μg/ml of chloramphenicol. At the same time, production of Escherichia coli strain SCS110-AF for the production of gene therapy DNA vector VTvaf17 or gene therapy DNA vectors based on it allowing for antibiotic-free positive selection involves constructing a 64 bp linear DNA fragment that contains regulatory element RNA-IN of transposon Tn10 allowing for antibiotic-free positive selection, a 1422 bp levansucrase gene sacB, the product of which ensures selection within a sucrose-containing medium, a 763 bp chloramphenicol resistance gene catR required for the selection of strain clones in which homologous recombination occurs, and two homologous sequences, 329 bp and 233 bp, ensuring homologous recombination in the region of gene recA concurrent with gene inactivation, and then the Escherichia coli cells are transformed by electroporation, and clones surviving in a medium containing 10 μg/ml of chloramphenicol are selected. The obtained strains for production were included in the collection of the National Biological Resource Centre—Russian National Collection of Industrial Microorganisms (NBRC RNCIM), RF and NCIMB Patent Deposit Service, UK under the following registration numbers: Escherichia coli strain SCS110-AF/VTvaf17-ANG—registered at the Russian National Collection of Industrial Microorganisms under number B-13280, date of deposit 16 Oct. 2018; INTERNATIONAL DEPOSITARY AUTHORITY No. NCIMB 43297, date of deposit 13 Dec. 2018; Escherichia coli strain SCS110-AF/VTvaf17-ANGPT1—registered at the Russian National Collection of Industrial Microorganisms under number B-13279, date of deposit 16 Oct. 2018; INTERNATIONAL DEPOSITARY AUTHORITY No. NCIMB 43300, date of deposit 13 Dec. 2018; Escherichia coli strain SCS110-AF/VTvaf17-VEGFA—registered at the Russian National Collection of Industrial Microorganisms under number B-13344, date of deposit 22 Nov. 2018; INTERNATIONAL DEPOSITARY AUTHORITY No. NCIMB 43289, date of deposit 22 Nov. 2018; Escherichia coli strain SCS110-AF/VTvaf17-FGF1—registered at the Russian National Collection of Industrial Microorganisms under number B-13338, date of deposit 22 Nov. 2018; INTERNATIONAL DEPOSITARY AUTHORITY No. NCIMB 43282, date of deposit 22 Nov. 2018; Escherichia coli strain SCS110-AF/VTvaf17-HIF1α-registered at the Russian National Collection of Industrial Microorganisms under number B-13383, date of deposit 14 Dec. 2018; INTERNATIONAL DEPOSITARY AUTHORITY No. NCIMB 43309, date of deposit 13 Dec. 2018; Escherichia coli strain SCS110-AF/VTvaf17-HGF—registered at the Russian National Collection of Industrial Microorganisms under number B-13260, date of deposit 24 Oct. 2018; INTERNATIONAL DEPOSITARY AUTHORITY No. NCIMB 43207, date of deposit 20 Sep. 2018; Escherichia coli strain SCS110-AF/VTvaf17-SDF1—registered at the Russian National Collection of Industrial Microorganisms under number B-13342, date of deposit 22 Nov. 2018; INTERNATIONAL DEPOSITARY AUTHORITY No. NCIMB 43287, date of deposit 22 Nov. 2018; Escherichia coli strain SCS110-AF/VTvaf17-KLK4—registered at the Russian National Collection of Industrial Microorganisms under number B-13346, date of deposit 22 Nov. 2018; INTERNATIONAL DEPOSITARY AUTHORITY No. NCIMB 43283, date of deposit 22 Nov. 2018; Escherichia coli strain SCS110-AF/VTvaf17-PDGFC—registered at the Russian National Collection of Industrial Microorganisms under number B-13340, date of deposit 22 Nov. 2018; INTERNATIONAL DEPOSITARY AUTHORITY No. NCIMB 43286, date of deposit 22 Nov. 2018; Escherichia coli strain SCS110-AF/VTvaf17-PROK1—registered at the Russian National Collection of Industrial Microorganisms under number B-13254, date of deposit 24 Oct. 2018; INTERNATIONAL DEPOSITARY AUTHORITY No. NCIMB 43209, date of deposit 20 Sep. 2018; Escherichia coli strain SCS110-AF/VTvaf17-PROK2—registered at the Russian National Collection of Industrial Microorganisms under number B-13261, date of deposit 24 Oct. 2018; INTERNATIONAL DEPOSITARY AUTHORITY No. NCIMB 43210, date of deposit 20 Sep. 2018;

### Example 41

A method of production of gene therapy DNA vector based on gene therapy DNA vector VTvaf17 carrying the therapeutic gene selected from the group of ANG, ANGPT1, VEGFA, FGF1, HIF1α, HGF, SDF1, KLK4, PDGFC, PROK1, and PROK2 genes on an industrial scale.

To confirm the producibility and constructability on an industrial scale of gene therapy DNA vector VTvaf17-ANG (SEQ ID No. 1), or VTvaf17-ANGPT1 (SEQ ID No. 2), or VTvaf17-VEGFA (SEQ ID No. 3), or VTvaf17-FGF1 (SEQ ID No. 4), or VTvaf17-HIF1α (SEQ ID No. 5), or VTvaf17-HGF (SEQ ID No. 6), or VTvaf17-SDF1 (SEQ ID No. 7), or VTvaf17-KLK4 (SEQ ID No. 8), or VTvaf17-PDGFC (SEQ ID No. 9), or VTvaf17-PROK1 (SEQ ID No. 10), or VTvaf17-PROK2 (SEQ ID No. 11), each carrying the therapeutic gene, namely ANG, or ANGPT1, or VEGFA, or FGF1, or HIF1α, or HGF, or SDF1, or KLK4, or PDGFC, or PROK1, or PROK2 gene, large-scale fermentation of Escherichia coli strain SCS110-AF/VTvaf17-ANG, or Escherichia coli strain SCS110-AF/VTvaf17-ANGPT1, or Escherichia coli strain SCS110-AF/VTvaf17-VEGFA, or Escherichia coli strain SCS110-AF/VTvaf17-FGF1, or Escherichia coli strain SCS110-AF/VTvaf17-HIF1α, or Escherichia coli strain SCS110-AF/VTvaf17-HGF, or Escherichia coli strain SCS110-AF/VTvaf17-SDF1, or Escherichia coli strain SCS110-AF/VTvaf17-KLK4, or Escherichia coli strain SCS110-AF/VTvaf17-PDGFC, or Escherichia coli strain SCS110-AF/VTvaf17-PROK1, or Escherichia coli strain SCS110-AF/VTvaf17-PROK2, each containing gene therapy DNA vector VTvaf17 carrying the therapeutic gene, namely ANG, or ANGPT1, or VEGFA, or FGF1, or HIF1α, or HGF, or SDF1, or KLK4, or PDGFC, or PROK1, or PROK2 gene, was performed. Escherichia coli strain SCS110-AF/VTvaf17-ANG, or Escherichia coli strain SCS110-AF/VTvaf17-ANGPT1, or Escherichia coli strain SCS110-AF/VTvaf17-VEGFA, or Escherichia coli strain SCS110-AF/VTvaf17-FGF1, or Escherichia coli strain SCS110-AF/VTvaf17-HIF1α, or Escherichia coli strain SCS110-AF/VTvaf17-HGF, or Escherichia coli strain SCS110-AF/VTvaf17-SDF1, or Escherichia coli strain SCS110-AF/VTvaf17-KLK4, or Escherichia coli strain SCS110-AF/VTvaf17-PDGFC, or Escherichia coli strain SCS110-AF/VTvaf17-PROK1, or Escherichia coli strain SCS110-AF/VTvaf17-PROK2 were constructed based on Escherichia coli strain SCS110-AF (Cell and Gene Therapy LLC, PIT Ltd) as described in Example 40 by electroporation of competent cells of this strain with the gene therapy DNA vector VTvaf17-ANG, or VTvaf17-ANGPT1, or VTvaf17-VEGFA, or VTvaf17-FGF1, or VTvaf17-HIF1α, or VTvaf17-HGF, or VTvaf17-SDF1, or VTvaf17-KLK4, or VTvaf17-PDGFC, or VTvaf17-PROK1, or VTvaf17-PROK2 carrying the therapeutic gene, namely ANG, or ANGPT1, or VEGFA, or FGF1, or HIF1α, or HGF, or SDF1, or KLK4, or PDGFC, or PROK1, or PROK2 gene, with further inoculation of transformed cells into agar plates (Petri dishes) with a selective medium containing yeastrel, peptone, and 6% sucrose, and selection of individual clones.

Fermentation of Escherichia coli SCS110-AF/VTvaf17-ANG carrying gene therapy DNA vector VTvaf17-ANG was performed in a 101 fermenter with subsequent extraction of gene therapy DNA vector VTvaf17-ANG.

For the fermentation of Escherichia coli strain SCS110-AF/VTvaf17-ANG, a medium was prepared containing (per 101 of volume): 100 g of tryptone, 50 g of yeastrel (Becton Dickinson), then the medium was diluted with water to 8800 ml and autoclaved at 121° C. for 20 minutes, and then 1200 ml of 50% (w/v) sucrose was added. After that, the seed culture of Escherichia coli strain SCS110-AF/VTvaf17-ANG was inoculated into a culture flask in the volume of 100 ml. The culture was incubated in an incubator shaker for 16 hours at 30° C. The seed culture was transferred to the Techfors S bioreactor (Infors HT, Switzerland) and grown to a stationary phase. The process was controlled by measuring optical density of the culture at 600 nm. The cells were pelleted for 30 minutes at 5,000-10,000 g. Supernatant was removed, and the cell pellet was re-suspended in 10% (by volume) phosphate buffered saline. The cells were centrifuged again for 30 minutes at 5,000-10,000 g. Supernatant was removed, a solution of 20 mM TrisCl, 1 mM EDTA, 200 g/l sucrose, pH 8.0 was added to the cell pellet in the volume of 1000 ml, and the mixture was stirred thoroughly to a homogenised suspension. Then egg lysozyme solution was added to the final concentration of 100 μg/ml. The mixture was incubated for 20 minutes on ice while stirring gently. Then 2500 ml of 0.2M NaOH, 10 g/l sodium dodecyl sulphate (SDS) was added, the mixture was incubated for 10 minutes on ice while stirring gently, then 3500 ml of 3M sodium acetate, 2M acetic acid, pH 5-5.5 was added, and the mixture was incubated for 10 minutes on ice while stirring gently. The resulting sample was centrifuged for 20-30 minutes at 15,000 g or a greater value. The solution was decanted delicately, and residual precipitate was removed by passing through a coarse filter (filter paper). Then RNase A (Sigma) was added to the final concentration of 20 μg/ml, and the solution was incubated overnight for 16 hours at room temperature. The solution was then centrifuged for 20-30 minutes at 15,000 g and passed through a 0.45p m membrane filter (Millipore). Then ultrafiltration was performed with a membrane of 100 kDa (Millipore) and the mixture was diluted to the initial volume with a buffer solution of 25 mM TrisCl, pH 7.0. This manipulation was performed three to four times. The solution was applied to the column with 250 ml of DEAE Sepharose HP (GE, USA), equilibrated with 25 mM TrisCl, pH 7.0. After the application of the sample, the column was washed with three volumes of the same solution and then gene therapy DNA vector VTvaf17-ANG was eluted using a linear gradient of 25 mM TrisCl, pH 7.0, to obtain a solution of 25 mM TrisCl, pH 7.0, 1M NaCl, five times the volume of the column. The elution process was controlled by measuring optical density of the run-off solution at 260 nm. Chromatographic fractions containing gene therapy DNA vector VTvaf17-ANG were joined together and subjected to gel filtration using Superdex 200 (GE, USA). The column was equilibrated with phosphate buffered saline. The elution process was controlled by measuring optical density of the run-off solution at 260 nm, and the fractions were analysed by agarose gel electrophoresis. The fractions containing gene therapy DNA vector VTvaf17-ANG were joined together and stored at −20° C. To assess the process reproducibility, the indicated processing operations were repeated five times. All processing operations for Escherichia coli strain SCS110-AF/VTvaf17-ANGPT1, or Escherichia coli strain SCS110-AF/VTvaf17-VEGFA, or Escherichia coli strain SCS110-AF/VTvaf17-FGF1, or Escherichia coli strain SCS110-AF/VTvaf17-HIF1α, or Escherichia coli strain SCS110-AF/VTvaf17-HGF, or Escherichia coli strain SCS110-AF/VTvaf17-SDF1, or Escherichia coli strain SCS110-AF/VTvaf17-KLK4, or Escherichia coli strain SCS110-AF/VTvaf17-PDGFC, or Escherichia coli strain SCS110-AF/VTvaf17-PROK1, or Escherichia coli strain SCS110-AF/VTvaf17-PROK2 were performed in a similar way.

The process reproducibility and quantitative characteristics of final product yield confirm the producibility and constructability of gene therapy DNA vector VTvaf17-ANG, or VTvaf17-ANGPT1, or VTvaf17-VEGFA, or VTvaf17-FGF1, or VTvaf17-HIF1α, or VTvaf17-HGF, or VTvaf17-SDF1, or VTvaf17-KLK4, or VTvaf17-PDGFC, or VTvaf17-PROK1, or VTvaf17-PROK2.

Thus, the produced gene therapy DNA vector containing the therapeutic gene can be used to deliver it to the cells of human beings and animals that experience reduced or insufficient expression of that gene, thus ensuring the desired therapeutic effect.

The purpose of this invention, namely the construction of a gene therapy DNA vector carrying the therapeutic human genes based on gene therapy DNA vector VTvaf17 for the treatment of diseases associated with the need to increase the expression level of these therapeutic genes that would reasonably combine:
I) possibility of safe use in the gene therapy of human beings and animals due to the absence of antibiotic resistance genes in the gene therapy DNA vector,
II) length that ensures efficient gene delivery to the target cell,
III) presence of regulatory elements that ensure efficient expression of the therapeutic genes while not being represented by nucleotide sequences of viral genomes,
IV) producibility and constructability on an industrial scale, as well as the purpose of construction of strains carrying these gene therapy DNA vectors for the production of these gene therapy DNA vectors on an industrial scale has been achieved, which is supported by the following examples:
for Item I—Example 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33; 34, 35, 36, 37, 38, 39, 40, 41;
for Item II—Example 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33; 34, 35, 36, 37, 38, 39, 40, 41;
for Item III—Example 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33; 34, 35, 36, 37, 38, 39, 40, 41;
for Item IV—Example 40, 41.

### INDUSTRIAL APPLICABILITY

All the examples listed above confirm the industrial applicability of the proposed gene therapy DNA vector based on gene therapy DNA vector VTvaf17 carrying the therapeutic gene selected from the group of ANG, ANGPT1, VEGFA, FGF1, HIF1α, HGF, SDF1, KLK4, PDGFC, PROK1, and PROK2 genes for increasing the expression level of these therapeutic genes, method of its production and use, Escherichia coli strain SCS110-AF/VTvaf17-ANG, or Escherichia coli strain SCS110-AF/VTvaf17-ANGPT1, or Escherichia coli strain SCS110-AF/VTvaf17-VEGFA, or Escherichia coli strain SCS110-AF/VTvaf17-FGF1, or Escherichia coli strain SCS110-AF/VTvaf17-HIF1α, or Escherichia coli strain SCS110-AF/VTvaf17-HGF, or Escherichia coli strain SCS110-AF/VTvaf17-SDF1, or Escherichia coli strain SCS110-AF/VTvaf17-KLK4, or Escherichia coli strain SCS110-AF/VTvaf17-PDGFC, or Escherichia coli strain SCS110-AF/VTvaf17-PROK1, or Escherichia coli strain SCS110-AF/VTvaf17-PROK2 carrying gene therapy DNA vector, method of its production, method of gene therapy DNA vector production on an industrial scale.

### List of Abbreviations

VTvaf17—Gene therapy vector devoid of sequences of viral genomes and antibiotic resistance markers (vector therapeutic virus-antibiotic-free)
DNA—Deoxyribonucleic acid
cDNA—Complementary deoxyribonucleic acid
RNA—Ribonucleic acid
mRNA—Messenger ribonucleic acid
bp—base pair
PCR—Polymerase chain reaction
RT-PCR—real-time PCR
ml—millilitre, μl—microlitre
mm3—cubic millimetre
l—litre
μg—microgram
mg—milligram
g—gram
μM—micromol
mM—millimol
min—minute
s—second
rpm—rotations per minute
nm—nanometre
cm—centimetre
mW—milliwatt
RFU—Relative fluorescence unit
PBS—Phosphate buffered saline

What is claimed is:

1.-28. (canceled)

29. A gene therapy DNA vector based on a gene therapy DNA vector VTvaf17 for treatment of diseases associated with disorders of tissue vascularisation, angiogenesis and hematopoiesis, hypoxia, disorders of various tissues regeneration, for treatment of diseases including ischemic damage to myocardium, brain, spinal cord, and limb muscle tissues, including in cases of diabetes, oncological and neurodegenerative diseases, including amyotrophic lateral sclerosis while the gene therapy DNA vector has a coding region of a therapeutic gene, selected from ANG, or ANGPT1, or VEGFA, or FGF1, or HIFIα, or HGF, or SDF1, or KLK4, or PDGFC, or PROK1, or PROK2, cloned to the gene therapy DNA vector VTvaf17 resulting in a gene therapy DNA vector VTvaf17-ANG that has a nucleotide sequence SEQ ID No. 1, or resulting in a gene therapy DNA vector VTvaf17-ANGPT1 that has a nucleotide sequence SEQ ID No. 2, or resulting in a gene therapy DNA vector VTvaf17-VEGFA that has a nucleotide sequence SEQ ID No. 3, or resulting in a gene therapy DNA vector VTvaf17-FGF1 that has a nucleotide sequence SEQ ID No. 4, or resulting in a gene therapy DNA vector VTvaf17-HIFIα that has a nucleotide sequence SEQ ID No. 5, or resulting in a gene therapy DNA vector VTvaf17-HGF that has a nucleotide sequence SEQ ID No. 6, or resulting in a gene therapy DNA vector VTvaf17-SDF1 that has a nucleotide sequence SEQ ID No. 7, or resulting in a gene therapy DNA vector VTvaf17-KLK4 that has a nucleotide sequence SEQ ID No. 8, or resulting in a gene therapy DNA vector VTvaf17-PDGFC that has a nucleotide sequence SEQ ID No. 9, or resulting in a gene therapy DNA vector VTvaf17-PROK1 that has a nucleotide sequence SEQ ID No. 10, or resulting in a gene therapy DNA vector VTvaf17-PROK2 that has a nucleotide sequence SEQ ID No.11 respectively.

30. The gene therapy DNA vector based on the gene therapy DNA vector VTvaf17 carrying ANG, or ANGPT1, or VEGFA, or FGF1, or HIFIα, or HGF, or SDF1, or KLK4, or PDGFC, or PROK1, or PROK2 therapeutic gene according to claim 29, the gene therapy DNA vectors being unique due to the fact that each of the constructed gene therapy DNA vectors: VTvafI7-ANG, or VTvafI7-ANGPT1, or VTvafI7-VEGFA, or VTvafI7-FGF1, or VTvafI7-HIF1α, or VTvafI7-HGF, or VTvafI7-SDF1, or VTvafI7-KLK4, or VTvafI7-PDGFC, or VTvafI7-PROK1, or VTvafI7-PROK2 uses nucleotide sequences that are not antibiotic resistance genes, virus genes, or regulatory elements of viral genomes as structure elements, which ensures a safe use for the gene therapy in humans and animals.

31. A method of gene therapy DNA vector production based on the gene therapy DNA vector VTvafI7 carrying the ANG, or ANGPT1, or VEGFA, or FGF1, or HIF1α, or HGF, or SDF1, or KLK4, or PDGFC, or PROK1, or PROK2 therapeutic gene according to claim 29 that includes obtaining each of the gene therapy DNA vectors VTvafI7-ANG, or VTvafI7-ANGPT1, or VTvafI7-VEGFA, or VTvafI7-FGF1, or VTvafI7-HIFIα, or VTvafI7-HGF, or VTvafI7-SDF1, or VTvafI7-KLK4, or VTvafI7-PDGFC, or VTvafI7-PROK1, or VTvafI7-PROK2 as follows: the coding region of the ANG, or ANGPT1, or VEGFA, or FGF1, or HIFIα, or HGF, or SDF1, or KLK4, or PDGFC, or PROK1, or PROK2 therapeutic gene is cloned to the gene therapy DNA vector VTvafI7, and the gene therapy DNA vector VTvafI7-ANG, SEQ ID No. 1, or VTvafI7-ANGPT1, SEQ ID No. 2, or VTvafI7-VEGFA, SEQ ID No. 3, or VTvafI7-FGFI, SEQ ID No. 4, or VTvafI7-HIFIα, SEQ ID No. 5, or VTvafI7-HGF, SEQ ID No. 6, or VTvafI7-SDF1, SEQ ID No. 7, or VTvafI7-KLK4, SEQ ID No. 8, or VTvafI7-PDGFC, SEQ ID No. 9, or VTvafI7-PROK1, SEQ ID No. 10, or VTvafI7-PROK2, SEQ ID No. 11, respectively, is obtained, wherein the coding region of the ANG, or ANGPT1, or VEGFA, or FGF1, or HIFIα, or HGF, or SDF1, or KLK4, or PDGFC, or PROK1, or PROK2 therapeutic gene is obtained by isolating a total RNA from a human biological tissue sample followed by a reverse transcription reaction and a PCR amplification using the obtained oligonucleotides and cleaving an amplification product by corresponding restriction endonucleases, wherein cloning to the gene therapy DNA vector VTvafI7 is performed by NheI [symbol] HindIII restriction sites, wherein the selection is performed without antibiotics,
at the same time, following oligonucleotides are used during the gene therapy DNA vector VTvafI7-ANG, SEQ ID No. 1 production for the reverse transcription reaction and the PCR amplification:
ANG_F
TTTGTCGACCACCATGGTGATGGGCCTGGGCGTT
ANG_R
AATGGTACCTTACGGACGACGGAAAATTGACTG,
and the cleaving of the amplification product and cloning of the coding region of ANG gene to gene therapy DNA vector VTvafI7 is performed by BamHI and EcoRI restriction endonucleases,
at the same time, following oligonucleotides are used during the gene therapy DNA vector VTvafI7-ANGPT1, SEQ ID No. 2 production for the reverse transcription reaction and the PCR amplification:
ANGPT1_F
TTTGTCGACCACCATGACAGTTTTCCTTTCCTTTGCTTTCC
ANGPT1_R
AATGGTACCTCAAAAATCTAAAGGTCGAATCATCATAGTTG,
and the cleaving of the amplification product and cloning of the coding region of ANGPT1 gene to the gene therapy DNA vector VTvafI7 is performed by SaiI and KpnI restriction endonucleases,
at the same time, following oligonucleotides are used during the gene therapy DNA vector VTvafI7-VEGFA, SEQ ID No. 3 production for the reverse transcription reaction and the PCR amplification:
VEGFA_F
GGGGGATCCACCATGACGGACAGACAGACAGACACCGC
VEGFA_R
TTTGGATCCACCATGAACTTTCTGCTGTCTTGGGTGC,
and the cleaving of the amplification product and cloning of the coding region of VEGFA gene to the gene therapy DNA vector VTvafI7 is performed by BamHI and HindIII restriction endonucleases,
at the same time, following oligonucleotides are used during the gene therapy DNA vector VTvafI7-FGFI, SEQ ID No. 4 production for the reverse transcription reaction and the PCR amplification:
FGF_F
TTTGTCGACCACCATGGCTGAAGGGGAAATCACC
FGF_R
AATGGTACCTTAATCAGAAGAGACTGGCAGGGG,
and the cleaving of the amplification product and cloning of the coding region of FGF1 gene to the gene therapy DNA vector VTvafI7 is performed by SaiI and KpnI restriction endonucleases,
at the same time, following oligonucleotides are used during the gene therapy DNA vector VTvafI7-HIFIα, SEQ ID No. 5 production for the reverse transcription reaction and the PCR amplification:
HIF_F
TTTGTCGACCACCATGGAGGGCGCCGGCGGCGCGA
HIF_R
AATGGTACCTCAGTTAACTTGATCCAAAGCTCTGAGTAATTC,
and the cleaving of the amplification product and cloning of the coding region of HIFIα gene to the gene therapy DNA vector VTvafI7 is performed by SaiI and KpnI restriction endonucleases,
at the same time, following oligonucleotides are used during the gene therapy DNA vector VTvafI7-HGF, SEQ ID No. 6 production for the reverse transcription reaction and the PCR amplification:
HGF_F
TTTGGATCCACCATGTGGGTGACCAAACTCCTGCCA
HGF_R
AATGTCGACCTATGACTGTGGTACCTTATATGTTAAAAT,
and the cleaving of the amplification product and cloning of the coding region of HGF gene to the gene therapy DNA vector VTvafI7 is performed by BamHI and SalI restriction endonucleases, at the same time, following oligonucleotides are used during the gene therapy DNA vector VTvafI7-SDFI, SEQ ID No. 7 production for the reverse transcription reaction and the PCR amplification:
SDF_F
AGGATCCCACCATGAACGCCAAGGTCGTGGT
SDF_R
TATGAATTCACATCTTGAACCTCTTGTTTAAAGC,
and the cleaving of the amplification product and cloning of the coding region of SDF1 gene to the gene therapy DNA vector VTvafI7 is performed by BamHI and EcoRI restriction endonucleases,
at the same time, following oligonucleotides are used during the gene therapy DNA vector VTvafI7-KLK4, SEQ ID No. 8 production for the reverse transcription reaction and the PCR amplification:
KLK_F
TTTGTCGACCACCATGGCCACAGCAGGAAATCCC
KLK_R
TTTTTGAATTCTTAACTGGCCTGGACGGTTTTCTC,
and the cleaving of the amplification product and cloning of the coding region of KLK4 gene to the gene therapy DNA vector VTvafI7 is performed by SalI and EcoRI restriction endonucleases,
at the same time, following oligonucleotides are used during gene the therapy DNA vector VTvafI7-PDGFC, SEQ ID No. 9 production for the reverse transcription reaction and the PCR amplification:
PDGFC_F
TTTGTCGACCACCATGAGCCTCTTCGGGCTTCTCC
PDGFC_R
AATGGTACCTATCCTCCTGTGCTCCCTCTGCAC,
and the cleaving of the amplification product and cloning of the coding region of PDGFC gene to the gene therapy DNA vector VTvafI7 is performed by SalI and KpnI restriction endonucleases,
at the same time, following oligonucleotides are used during the gene therapy DNA vector VTvafI7-PROKI, SEQ ID No. 10 production for the reverse transcription reaction and the PCR amplification:
PROK1_F
TATGTCGACCACCATGAGAGGTGCCACGCGAG
PROK1_R
TATGGAATTCGGTACGCTAAAAATTGATGTTCTTCAAGTCCA,
and the cleaving of the amplification product and cloning of the coding region of PROK1 gene to the gene therapy DNA vector VTvafI7 is performed by SalI and EcoRI restriction endonucleases,
at the same time, following oligonucleotides are used during the gene therapy DNA vector VTvafI7-PROK2, SEQ ID No. 11 production for the reverse transcription reaction and the PCR amplification:
PROK2_F
TTTGTCGACCACCATGAGGAGCCTGTGCTGCG
PROK2_R
AATGGTACCTTACTTTTGGGCTAAACAAATAAATCGG,
and the cleaving of the amplification product and cloning of the coding region of PROK2 gene to the gene therapy DNA vector VTvafI7 is performed by SalI and KpnI restriction endonucleases.

32. A method of use of the gene therapy DNA vector based on gene therapy DNA vector VTvafI7 carrying ANG, or ANGPT1, or VEGFA, or FGF1, or HIFIα, or HGF, or SDF1, or KLK4, or PDGFC, or PROK1, or PROK2 therapeutic gene according to claim 29 for treatment of diseases associated with disorders of tissue vascularisation, angiogenesis and hematopoiesis, hypoxia, disorders of various tissues regeneration, for treatment of diseases including ischemic damage to myocardium, brain, spinal cord, and limb muscle tissues, including in cases of diabetes, oncological and neurodegenerative diseases, including amyotrophic lateral sclerosis that involves transfection of cells of patient or animal organs and tissues with the selected gene therapy DNA vector carrying the therapeutic gene based on the gene therapy DNA vector VTvafI7, or several selected gene therapy DNA vectors carrying therapeutic genes based on the gene therapy DNA vector VTvafI7, from a group of the constructed gene therapy DNA vectors carrying the therapeutic genes based on the gene therapy DNA vector VTvafI7 and injection of autologous cells of the patient or animal transfected by the selected gene therapy DNA vector carrying the therapeutic gene based on the gene therapy DNA vector VTvafI7 or several selected gene therapy DNA vectors carrying the therapeutic genes based on the gene therapy DNA vector VTvafI7 from the constructed gene therapy DNA vectors carrying the therapeutic genes based on the gene therapy DNA vector VTvafI7 into organs and tissues of the same patient or animal and the injection of the selected gene therapy DNA vector carrying the therapeutic gene based on the gene therapy DNA vector VTvafI7 or several selected gene therapy DNA vectors carrying the therapeutic genes based on the gene therapy DNA vector VTvafI7 from the group of the constructed gene therapy DNA vectors carrying the therapeutic genes based on the gene therapy DNA vector VTvafI7 into the organs and tissues of the same patient or animal, or the combination of the indicated methods.
